(19) United States
(12) Patent Application Publication
(10) Pub. No.: US 2026/0151383 A1
(43) Pub. Date: Jun. 4, 2026

(54) HETEROCYCLIC COMPOUNDS AND USES THEREOF

(71) Applicant: KayoThera Inc., Seattle, WA (US)

(72) Inventors: Mark ESPOSITO, Cazenovia, NY (US); John PROUDFOOT, Newtown, CT (US); John PIWINSKI, Lebanon, NJ (US)

(21) Appl. No.: 19/122,953

(22) PCT Filed: Oct. 24, 2023
(86) PCT No.: PCT/US2023/077659

§ 371 Date: Apr. 21, 2025

Related U.S. Application Data
(60) Provisional application No. 63/418,858, filed on Oct. 24, 2022.
(60) Provisional application No. 63/517,467, filed on Aug. 3, 2023.

(51) Int. Cl. A61K 31/4709; A61K 31/4704; A61K 38/20; A61K 39/395; A61K 40/11; A61K 45/06; C07D 215/22; C07D 401/12; C07D 417/12

(52) U.S. Cl. CPC A61K 31/4709; A61K 31/4704; A61K 38/2013; A61K 39/3955; A61K 40/11; A61K 45/06; C07D 215/22; C07D 401/12; C07D 417/12

(57) ABSTRACT

Provided herein are compounds of Formula (I), or pharmaceutically acceptable salt thereof, wherein R¹⁰⁰, p, ring A, L, R^{22′}, R²², R³², and R³³ are defined herein. Also provided herein are pharmaceutical compositions comprising a compound of Formula (I) or pharmaceutically acceptable salt thereof, and methods of using a compound of Formula (I) or pharmaceutically acceptable salt thereof, e.g., in the treatment of a disease or disorder that is treatable by administration of an aldehyde dehydrogenase (e.g., ALDH1a3 and/or ALDH1a2) inhibitor.

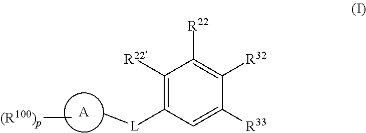

### CROSS-REFERENCE TO RELATED APPLICATIONS

This application claims priority to U.S. Provisional Application No. 63/517,467, filed Aug. 3, 2023, and U.S. Provisional Application No. 63/418,858, filed Oct. 24, 2022, the contents of which are hereby incorporated by reference in their entireties for all purposes.

### BACKGROUND

Aldehyde dehydrogenases (ALDHs) belong to a superfamily of NAD(P+)-dependent enzymes that play a role in the metabolism of aldehydes by irreversibly catalyzing the oxidation of both endogenously and exogenously produced aldehydes to their respective carboxylic acids. ALDHs have a broad spectrum of biological activities, including biosynthesis of retinoic acid (RA), oxidation of lipid peroxides, and alcohol metabolism, among others.

The ALDH family of enzymes contains 19 members with diverse functions. Enzymes within this family irreversibly catalyze the oxidation of an aldehyde into the corresponding carboxylic acid while reducing NAD+/NADP+ to NADH/NADPH. These enzymes are found in several cellular compartments, however, most are localized to the cytosol or the mitochondria.

The ALDH enzymes can be classified into 3 main groups: 1) Broad specificity ALDH1 family containing ALDH1a1, 1a2, 1a3, 1b1 and ALDH2 known to oxidize retinal, acetaldehyde, GABA, medium chain lipid aldehydes, cyclophosphamide metabolites and potentially other aldehyde species; 2) Long chain fatty aldehyde-specific ALDH3A1-3B2; and 3) Structurally diverse enzymes ALDH4A1, 5A1, 6A1, 7A1, 8A1, 9A1,18A1, 1L1, and 1L2 catalyze the oxidation of discrete metabolites or semialdehydes (glutamate, succinate, methylmalonate, 10-formyl-THF etc.).

### SUMMARY

Some ALDH enzymes participate in global metabolism via expression in the liver where they function to detoxify acetylaldehyde formed from alcohol dehydrogenases, biosynthesize retinoic acid from retinal stereoisomers, or detoxify other reactive aldehydes. In contrast, most ALDH enzymes are expressed in a cell- or disease-specific manner and modulate cellular biochemistry, often with unknown mechanisms of action.

Of particular interest to human health and disease is the ALDH1a subfamily; ALDH1a3 is distinctly upregulated across solid tumor subpopulations, failing pancreatic s cells, and proliferative smooth muscle cells in endothelial diseases. ALDH1a3 is further used to identify tumorigenic and chemoresistant cancer cells. Studies have shown that tumor-expressed ALDH1a3 conditions the tumor microenvironment through the generation of paracrine retinoic acid that suppresses anti-tumor immunity.

A less studied but equally important ALDH1a enzyme is ALDH1a2, which has been implicated as the main myeloid-expressed ALDH1a isoform. Similar to ALDH1a3, ALDH1a2 catalyzes retinaldehyde oxidation, and this activity controls normal immune tolerance through the induction of Regulatory T cells and M2 macrophages. However, few studies have been performed on the role of ALDH1a2 in controlling immune tolerance of cancer or other pathologies.

While insights into the complementary roles of ALDH1a2 and ALDH1a3 in conditioning an immune suppressive niche are novel, retinoid signaling via the RAR nuclear receptors is arguably the most studied nutrient pathway in inflammatory diseases and cancer. Early trials established retinoic acid as a tremendously effective intervention in acute promyelocytic leukemia (APL) through targeting a fusion oncogene unique to APL. However, numerous follow-on clinical trials testing retinoid agonism in solid tumors unexpectedly revealed a pro-tumorigenic role of retinoid signaling across solid tumor types, resulting in numerous early terminations due to excess mortality and increased cancer incidence. Therapeutic retinoid agonism is also associated with hyperlipidemia, osteoarthritis, and various other pathologies caused by immune suppression.

The present disclosure is based, in part, on the discovery that various compounds described herein can potently and/or selectively inhibit one or more ALDH enzymes such as ALDH1a3 and/or ALDH1a2, can inhibit the retinoid pathway activation, and can treat various diseases such as cancers, cancer metastasis, and other ALDH1a2 and/or ALDH1a3-mediated diseases and disorders, metabolic diseases, such as such as type 2 diabetes, pulmonary arterial hypertension (PAH) and neointimal hyperplasia (NIH).

In embodiments, the present disclosure provides a compound of Formula (I):

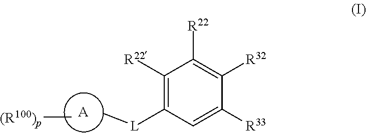

or a pharmaceutically acceptable salt thereof,
wherein:
ring A is a heterocyclyl, heteroaryl, or aryl;
L is —NH—, —C(O)—NH—, —C(O)—N(R^{A})-alkylene-O—, -(alkylene)-N(R^{A})-alkylene-O—, —S(O)NH—, —S(O)₂NH—, —S(O)—, or —S(O)₂—,
R²² is halo, —CO—N(R^{A})₂, —CN, —C₁₋₆ alkyl, —C₁₋₆ alkyl-CN, —C₁₋₆ haloalkyl, or carbocyclyl;
R^{22′} is H, —CN, halo, —C₁₋₆ alkyl, or —C₁₋₆ haloalkyl; or
R²² and R^{22′} are joined to form a heteroaryl, carbocyclyl, or heterocyclyl, each of which may be substituted with one or more halo;
R³² and R³³ are joined to form a heterocyclyl and substituted with oxo; and wherein the heterocyclyl may be further optionally substituted with one or more R¹⁰¹;
p is 0, 1 or 2;
each R¹⁰⁰ is independently halo, —CN, hydroxy, —C₁₋₆ alkyl, —C₁₋₆ alkylene-carbocyclyl, —C₁₋₆ alkylene-heterocyclyl, —C₁₋₆ haloalkyl, carbocyclyl, aryl, or heteroaryl, wherein aryl or heteroaryl is optionally substituted with one or more R¹⁰¹;
each R¹⁰¹ is independently halo, —CN, —CO—N(R^{A})₂, hydroxy, —C₁₋₆ alkyl, or —C₁₋₆ haloalkyl; and R^{A} is H, —C₁₋₆ alkyl, or aryl,
   wherein when L is —C(O)—NH— or —S(O)₂NH—, and A is aryl, then p is 1 or 2 and at least one 1 R¹⁰⁰ is aryl optionally substituted with one or more R¹⁰¹, and wherein the compound is not

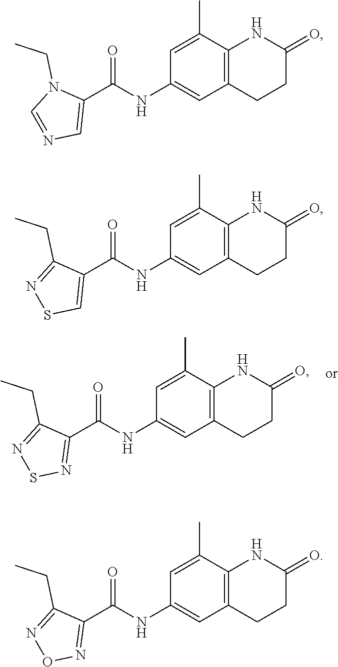 [SMILES: CCn1cncc1C(=O)Nc1cc(C)c2c(c1)CCC(=O)N2 ; CCc1nscc1C(=O)Nc1cc(C)c2c(c1)CCC(=O)N2 ; CCc1nsnc1C(=O)Nc1cc(C)c2c(c1)CCC(=O)N2 ; CCc1nonc1C(=O)Nc1cc(C)c2c(c1)CCC(=O)N2]

In embodiments, the compound of Formula (I) is a compound of Formula (III)

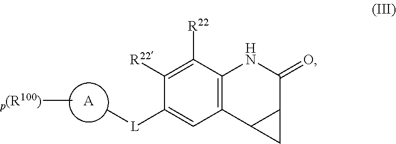

or a pharmaceutically acceptable salt or deuterated form thereof,
   wherein:
ring A is heteroaryl, heterocyclyl, or aryl;
L is —C(O)—NH—, —C(NR^{A})—NH—, —C(O)—N(R^{A})-alkylene-O—, or -(alkylene)-N(R^{A})-alkylene-O—;
R²² is halo, —CN, —C₁₋₆ alkyl, —C₁₋₆ alkyl-CN, —C₁₋₆ haloalkyl, -carbocyclyl, or —C₁₋₆ alkyl-carbocyclyl;
R^{22′} is H, —CN, halo, —C₁₋₆ alkyl, —C₁₋₆ haloalkyl, —C₁₋₆ alkyl-CN, carbocyclyl, or —C₁₋₆ alkyl-carbocyclyl;
p is 0, 1, or 2;
each R¹⁰⁰ is independently halo, —CN, hydroxy, —C₁₋₆ alkyl, —C₁₋₆ alkylene-carbocyclyl, —C₁₋₆ alkylene-heterocyclyl, —C₁₋₆ haloalkyl, carbocyclyl, aryl, or heteroaryl, wherein aryl or heteroaryl is optionally substituted with 1 or 2 R¹⁰¹;
each R¹⁰¹ is independently halo, —CN, hydroxy, or —C₁₋₆ alkyl; and
   R^{A} is H or —C₁₋₆ alkyl; and
   wherein the compound is not

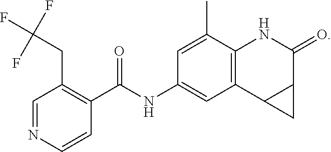 [SMILES: Cc1cc(NC(=O)c2ccncc2CC(F)(F)F)cc2c1NC(=O)C1CC21]

In embodiments, the compound of Formula (III) is a compound of Formula (IIIA):

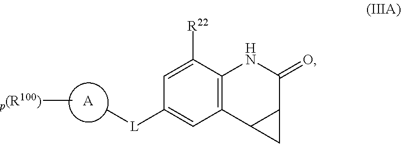

or a pharmaceutically acceptable salt or deuterated form thereof,
   wherein:
ring A is heteroaryl or aryl;
L is —C(O)—NH—, —C(NR^{A})—NH—, —C(O)—N(R^{A})-alkylene-O—, or -(alkylene)-N(R^{A})-alkylene-O—;
R²² is halo, —CN, —C₁₋₆ alkyl, —C₁₋₆ alkyl-CN, —C₁₋₆ haloalkyl, -carbocyclyl, or —C₁₋₆ alkyl-carbocyclyl;
p is 0, 1, or 2;
each R¹⁰⁰ is independently halo, —C₁₋₆ alkyl, —C₁₋₆ alkylene-carbocyclyl, —C₁₋₆ alkylene-heterocyclyl, —C₁₋₆ haloalkyl, or aryl, wherein aryl is optionally substituted with 1 or 2 R¹⁰¹;
each R¹⁰¹ is independently halo, —CN, hydroxy, or —C₁₋₆ alkyl; and
R^{A} is H or —C₁₋₆ alkyl,
   wherein the compound is not

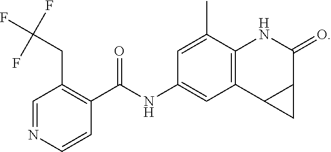 [SMILES: Cc1cc(NC(=O)c2ccncc2CC(F)(F)F)cc2c1NC(=O)C1CC21]

In embodiments, the compound of Formula (I) is a compound of Formula (IV):

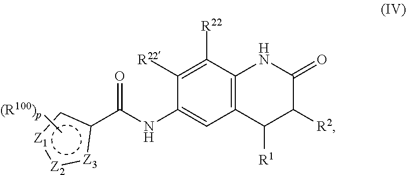

or a pharmaceutically acceptable salt or deuterated form thereof,
   wherein:
R¹ is H and R² is H, or R¹ and R² are joined to form a carbocyclyl;
R²² is halo, —CN, —C₁₋₆alkyl, —C₁₋₆ alkyl-CN, —C₁₋₆haloalkyl, carbocyclyl, or —C₁₋₆ alkyl-carbocyclyl;
R²² is H, —CN, halo, —C₁₋₆ alkyl, —C₁₋₆ haloalkyl, —C₁₋₆ alkyl-CN, carbocyclyl, or —C₁₋₆ alkyl-carbocyclyl;
p is 0, 1, or 2;
each R¹⁰⁰ is independently halo, —CN, hydroxy, —C₁₋₆ alkylene-carbocyclyl, —C₁₋₆ alkyl, —C₁₋₆ haloalkyl, or carbocyclyl;
Z₁, Z₂, and Z₃ are independently selected from —CH—, —S—, —N—, —NH—, and —O—; and
the dashed line represents a double or is absent to provide an aromatic ring;
wherein the compound is not

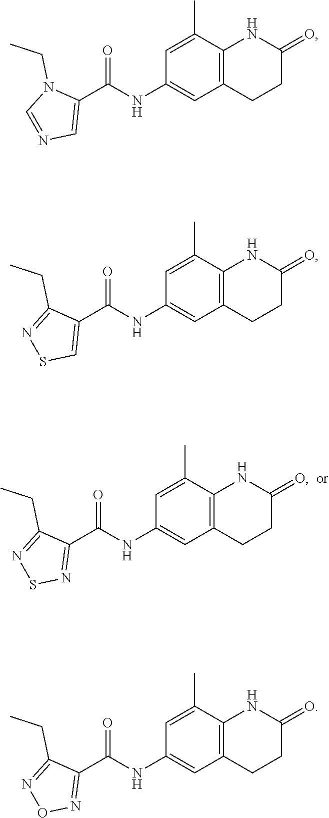

In embodiments, the compound of Formula (IV) is a compound of Formula (IVA):

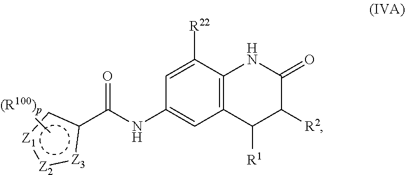

or a pharmaceutically acceptable salt or deuterated form thereof,
   wherein:
R¹ is H and R² is H, or R¹ and R² are joined to form a carbocyclyl;
R²² is halo, —CN, —C₁₋₆alkyl, —C₁₋₆ alkyl-CN, —C₁₋₆ haloalkyl, carbocyclyl, or —C₁₋₆ alkyl-carbocyclyl;
p is 0, 1, or 2;
each R¹⁰⁰ is independently halo, —CN, hydroxy, —C₁₋₆ alkylene-carbocyclyl, —C₁₋₆ alkyl, —C₁₋₆ haloalkyl, or carbocyclyl;
Z₁, Z₂, and Z₃ are independently selected from CH, S, N, NH, and O; and
dashed line represents a double or is absent to provide an aromatic ring,
   wherein the compound is not

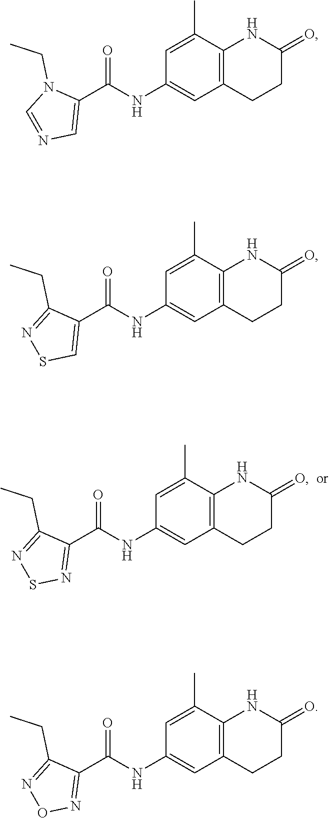

In embodiments, provided herein is a pharmaceutical composition comprising a compound of the present disclosure (e.g., a compound of Formula (I)-(IVA), or Table 1), or a pharmaceutically acceptable salt thereof, and a pharmaceutically acceptable carrier.

In embodiments, provided herein are methods of treating cancer in a subject in need thereof, the method comprising administering to the subject a therapeutically effective amount of the compound of the present disclosure (e.g., a compound of Formula (I)-(IVA), or Table 1), or a pharmaceutically acceptable salt thereof, or the pharmaceutical composition of the present disclosure comprising a compound of the present disclosure (e.g., a compound of Formula (I), (II), or Table 1), or a pharmaceutically acceptable salt thereof, and a pharmaceutically acceptable carrier.

In embodiments, provided herein are methods for treating or preventing type 2 diabetes in a subject in need thereof, the method comprising administering to the subject a therapeutically effective amount of the compound of the present disclosure (e.g., a compound of Formula (I)-(IVA), or Table 1), or a pharmaceutically acceptable salt thereof, or the pharmaceutical composition of the present disclosure comprising a compound of the present disclosure (e.g., a compound of Formula (I)-(IVA), or Table 1), or a pharmaceutically acceptable salt thereof, and a pharmaceutically acceptable carrier.

In embodiments, provided herein are methods for treating or preventing a metabolic disease in a subject in need thereof, the method comprising administering to the subject a therapeutically effective amount of the compound of the present disclosure (e.g., a compound of Formula (I)-(IVA), or Table 1), or a pharmaceutically acceptable salt thereof, or the pharmaceutical composition of the present disclosure comprising a compound of the present disclosure (e.g., a compound of Formula (I)-(IVA), or Table 1), or a pharmaceutically acceptable salt thereof, and a pharmaceutically acceptable carrier.

In embodiments, provided herein are methods for inhibiting an aldehyde dehydrogenase (e.g., ALDH1a3 and/or ALDH1a2) in a subject in need thereof, the method comprising administering to the subject a therapeutically effective amount of the compound of the present disclosure (e.g., a compound of Formula (I)-(IVA), or Table 1), or a pharmaceutically acceptable salt thereof, or the pharmaceutical composition of the present disclosure comprising a compound of the present disclosure (e.g., a compound of Formula (I)-(IVA), or Table 1), or a pharmaceutically acceptable salt thereof, and a pharmaceutically acceptable carrier.

In embodiments, provided herein are methods for treating a disease or disorder associated with aldehyde dehydrogenase (e.g., ALDH1a3 and/or ALDH1a2) in a subject in need thereof, the method comprising administering to the subject a therapeutically effective amount of the compound of the present disclosure (e.g., a compound of Formula (I)-(IVA), or Table 1), or a pharmaceutically acceptable salt thereof, or the pharmaceutical composition of the present disclosure comprising a compound of the present disclosure (e.g., a compound of Formula (I)-(IVA), or Table 1), or a pharmaceutically acceptable salt thereof, and a pharmaceutically acceptable carrier.

In embodiments, provided herein are methods for treating an endothelial cell or smooth muscle cell disease or disorder in a subject in need thereof, the method comprising administering to the subject a therapeutically effective amount of the compound of the present disclosure (e.g., a compound of Formula (I)-(IVA), or Table 1), or a pharmaceutically acceptable salt thereof, or the pharmaceutical composition of the present disclosure comprising a compound of the present disclosure (e.g., a compound of Formula (I)-(IVA), or Table 1), or a pharmaceutically acceptable salt thereof, and a pharmaceutically acceptable carrier.

In embodiments, provided herein are methods for antagonizing the retinoid pathway in a subject in need thereof, the method comprising administering to the subject a therapeutically effective amount of the compound of the present disclosure (e.g., a compound of Formula (I)-(IVA), or Table 1), or a pharmaceutically acceptable salt thereof, or the pharmaceutical composition of the present disclosure comprising a compound of the present disclosure (e.g., a compound of Formula (I)-(IVA), or Table 1), or a pharmaceutically acceptable salt thereof, and a pharmaceutically acceptable carrier.

In embodiments, provided herein are methods for male contraception in a subject in need thereof, the method comprising administering to the subject a therapeutically effective amount of the compound of the present disclosure (e.g., a compound of Formula (I)-(IVA), or Table 1), or a pharmaceutically acceptable salt thereof, or the pharmaceutical composition of the present disclosure comprising a compound of the present disclosure (e.g., a compound of Formula (I)-(IVA), or Table 1), or a pharmaceutically acceptable salt thereof, and a pharmaceutically acceptable carrier.

### BRIEF DESCRIPTION OF THE FIGURES

FIG. 1. Expression of ALDH1a2 and ALDH1a3 mRNA in patient-derived xenograft models from multiple human cancers shows expression of ALDH1a2 and ALDH1a3 enzymes across multiple tumor types.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure.

This disclosure will govern in the instance that there is any inconsistency between the patents, patent applications and publications cited and this disclosure.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range of plus or minus an acceptable degree of variation in the art. In embodiments, the term “about” encompasses 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆alkyl.

“Halo” or “halogen” refers to fluorine (fluoro, —F), chlorine (chloro, —Cl), bromine (bromo, —Br), or iodine (iodo, —I).

The term “pharmaceutically acceptable salt” refers to those salts which are suitable for use in contact with the tissues of humans and animals without undue toxicity, irritation, allergic response, and the like, and are commensurate with a reasonable benefit/risk ratio, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

As used herein, the term “alkyl” as used by itself or as part of another group refers to a straight- or branched-chain aliphatic saturated hydrocarbon. In embodiments, the alkyl which can include one to twelve carbon atoms (i.e., C₁₋₁₂ alkyl) or the number of carbon atoms designated. In embodiments, the alkyl group is a straight chain C₁₋₁₀ alkyl group. In embodiments, the alkyl group is a branched chain C₃₋₁₀ alkyl group. In embodiments, the alkyl group is a straight chain C₁₋₆ alkyl group. In embodiments, the alkyl group is a branched chain C₃₋₆ alkyl group. In embodiments, the alkyl group is a straight chain C₁₋₄ alkyl group. For example, a C₁₋₄ alkyl group as used herein refers to a group selected from methyl, ethyl, n-propyl, i-propyl, sec-propyl, n-butyl, i-butyl, sec-butyl, I-butyl, n-pentyl, t-amyl, n-hexyl, n-heptyl, n-octyl, n-nonyl, n-decyl, n-undecyl, and n-dodecyl. In embodiments, “alkyl” is a straight-chain hydrocarbon. In some embodiments, “alkyl” is a branched hydrocarbon. Unless stated otherwise specifically in the specification, an alkyl group can be optionally substituted.

“Alkylene” or “alkylene chain” refers to a fully saturated, straight or branched divalent hydrocarbon chain radical. In embodiments, the alkylene has from one to twelve carbon atoms. Non-limiting examples of C₁-C₁₂ alkylene include methylene, ethylene, propylene, n-butylene, ethenylene, propenylene, n-butenylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

As used herein, the term “alkenyl” as used by itself or as part of another group refers to a straight- or branched-chain aliphatic hydrocarbon containing one or more, for example, one, two or three carbon-to-carbon double bonds. In embodiments, the alkenyl group is a C₂₋₆ alkenyl group. In embodiments, the alkenyl group is a C₂₋₄ alkenyl group. Non-limiting exemplary alkenyl groups include ethenyl, propenyl, isopropenyl, butenyl, sec-butenyl, pentenyl, and hexenyl. Unless stated otherwise specifically in the specification, an alkenyl group can be optionally substituted.

As used herein, the term “alkynyl” as used by itself or as part of another group refers to a straight- or branched-chain aliphatic hydrocarbon containing one or more, for example, one to three carbon-to-carbon triple bonds. In embodiments, the alkynyl has one carbon-carbon triple bond. In one embodiment, the alkynyl group is a C₂₋₆ alkynyl group. In embodiments, the alkynyl group is a C₂₋₄ alkynyl group. Non-limiting exemplary alkynyl groups include ethynyl, propynyl, butynyl, 2-butynyl, pentynyl, and hexynyl groups. Unless stated otherwise specifically in the specification, an alkynyl group can be optionally substituted.

As used herein, the term “alkoxy” as used by itself or as part of another group refers to a radical of the formula OR^{a1}, wherein R^{a1} is an alkyl, alkenyl, or alkynyl as defined herein. In embodiments, R^{a1} is an alkyl. Unless stated otherwise specifically in the specification, an alkoxy group can be optionally substituted.

“Carbocyclyl,” “carbocyclic ring” or “carbocycle” refers to a rings structure, wherein the atoms which form the ring are each carbon, and which is attached to the rest of the molecule by a single bond. Carbocyclic rings can comprise from 3 to 20 carbon atoms in the ring. Carbocyclic rings include aryls and cycloalkyl, cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon consisting solely of carbon and hydrogen atoms, which can include fused, spirocyclic, or bridged ring systems (e.g., fused, or bridged ring systems), having from three to twenty carbon atoms, and which is attached to the rest of the molecule by a single bond. Monocyclic cycloalkyl include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl include, for example, cyclopentenyl, cyclohexenyl, cycloheptenyl, cyclooctenyl, and the like. Polycyclic cycloalkenyls include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon consisting solely of carbon and hydrogen atoms, having from 3 to 20 carbon atoms and one or more carbon-carbon triple bonds, which can include fused or bridged ring systems, and which is attached to the rest of the molecule by a single bond. Monocyclic cycloalkynyls include, for example, cycloheptynyl, cyclooctynyl, and the like. Unless otherwise stated specifically in the specification, a cycloalkynyl group can be optionally substituted.

As used herein, the term “cycloalkoxy” as used by itself or as part of another group refers to a radical of the formula OR^{a1}, wherein R^{a1} is a cycloalkyl as defined herein. Unless stated otherwise specifically in the specification, a cycloalkoxy group can be optionally substituted.

As used herein, the term “haloalkyl” as used by itself or as part of another group refers to an alkyl substituted with one or more fluorine, chlorine, bromine and/or iodine atoms. In embodiments, the haloalkyl is an alkyl group substituted with one, two, or three fluorine atoms. In one embodiment, the haloalkyl group is a C₁₋₁₀ haloalkyl group. In embodiments, the haloalkyl group is a C₁₋₆ haloalkyl group. In embodiments, the haloalkyl group is a C₁₋₄ haloalkyl group. Unless otherwise stated specifically in the specification, a haloalkyl group can be optionally substituted.

“Heterocyclyl” or “heterocyclic” or “heterocycle” as used by itself or as part of another group refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is on the heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system. Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like. Unless otherwise stated specifically in the specification, a heterocyclyl can be optionally substituted.

“Aryl” as used by itself or as part of another group refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₄ aryl”). In embodiments, an aryl group has six ring carbon atoms (“C₆ aryl”; e.g., phenyl). In embodiments, an aryl group has ten ring carbon atoms (“C₁₀ aryl”; e.g., naphthyl such as 1-naphthyl and 2-naphthyl). In embodiments, an aryl group has fourteen ring carbon atoms (“C₁₋₄aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise stated specifically in the specification, an aryl can be optionally substituted.

“Aralkyl” as used by itself or as part of another group refers to an alkyl substituted with one or more aryl groups, preferably, substituted with one aryl group. Examples of aralkyl include benzyl, phenethyl, etc. When an aralkyl is said to be optionally substituted, either the alkyl portion or the aryl portion of the aralkyl can be optionally substituted.

“Heteroaryl” as used by itself or as part of another group refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, and the like) the point of attachment can be on either ring, i.e., either the ring bearing a heteroatom (e.g., 2-indolyl) or the ring that does not contain a heteroatom (e.g., 5-indolyl). Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Unless otherwise stated specifically in the specification, a heteroaryl can be optionally substituted.

“Heteroaralkyl” as used by itself or as part of another group refers to an alkyl substituted with one or more heteroaryl groups, preferably, substituted with one heteroaryl group. When a heteroaralkyl is said to be optionally substituted, either the alkyl portion or the heteroaryl portion of the heteroaralkyl can be optionally substituted.

An “optionally substituted” group as used herein means any of the above groups, such as an optionally substituted alkyl, optionally substituted alkenyl, optionally substituted alkynyl, optionally substituted carbocyclyl, optionally substituted cycloalkyl, optionally substituted cycloalkenyl, optionally substituted cycloalkynyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable. Two of the optional substituents can join to form an optionally substituted cycloalkyl, heterocylyl, aryl, or heteroaryl ring. Substitution can occur on any available carbon, oxygen, or nitrogen atom, and can form a spirocycle. Typically, substitution herein does not result in an O—O, O—N, S—S, S—N(except SO₂—N bond), heteroatom-halogen, or —C(O)—S bond or three or more consecutive heteroatoms, with the exception of O—SO₂—O, O—SO₂—N, and N—SO₂—N, except that some of such bonds or connections may be allowed if in a stable aromatic system.

In a broad aspect, the permissible substituents herein include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a cycloalkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aryl, or a heteroaryl, each of which can be substituted, if appropriate.

Exemplary substituents include, but not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, cycloalkyl, heterocycloalkyl, —O—C(O)-alkyl, —O—C(O)-aryl, —O—C(O)-cycloalkyl, —C(—N—CN)—NH₂, —C(—NH)—NH₂, —C(—NH)—NH(alkyl), —N(Y₁)(Y₂), -alkylene-N(Y₁)(Y₂), —C(O)N(Y₁)(Y₂), and —S(O)₂N(Y₁)(Y₂), wherein Yⱼ and Y₂ can be the same or different and are independently selected from the group consisting of hydrogen, alkyl, aryl, cycloalkyl, and -alkylene-aryl.

Some examples of suitable substituents include, but not limited to, (C₁-C₈)alkyl groups, (C₂-C₈)alkenyl groups, (C₂-C₈)alkynyl groups, (C₃-C₁₀)cycloalkyl groups, halogen (F, Cl, Br or I), halogenated (C₁-C₈)alkyl groups (for example but not limited to —CF₃), —O—(C₁-C₈)alkyl groups, —OH, —S—(C₁-C₈)alkyl groups, —SH, —NH(C₁-C₈)alkyl groups, —N((C₁-C₈)alkyl)₂ groups, —NH₂, —C(O)NH₂, —C(O)NH(C₁-C₈)alkyl groups, —C(O)N((C₁-C₈)alkyl)₂, —NHC(O)H, —NHC(O) (C₁-C₈)alkyl groups, —NHC(O) (C₃-C₈)cycloalkyl groups, —N((C₁-C₈)alkyl)C(O)H, —N((C₁-C₈)alkyl)C(O)(C₁-C₈)alkyl groups, —NHC(O)NH₂, —NHC(O)NH(C₁-C₈)alkyl groups, —N((C₁-C₈)alkyl)C(O)NH₂ groups, —NHC(O)N((C₁-C₈)alkyl)₂ groups, —N((C₁-C₈)alkyl)C(O)N((C₁-C₈)alkyl)₂ groups, —N((C₁-C₈)alkyl)C(O)NH((C₁-C₈)alkyl), —C(O)H, —C(O)(C₁-C₈)alkyl groups, —CN, —NO₂, —S(O)(C₁-C₈)alkyl groups, —S(O)₂(C₁-C₈)alkyl groups, —S(O)₂N((C₁-C₈)alkyl)₂ groups, —S(O)₂NH(C₁-C₈)alkyl groups, —S(O)₂NH(C₃-C₈)cycloalkyl groups, —S(O)₂NH₂ groups, —NHS(O)₂(C₁-C₈)alkyl groups, —N((C₁-C₈)alkyl)S(O)₂(C₁-C₈)alkyl groups, —(C₁-C₈)alkyl-O—(C₁-C₈)alkyl groups, —O—(C₁-C₈)alkyl-O—(C₁-C₈)alkyl groups, —C(O)OH, —C(O)O(C₁-C₈)alkyl groups, NHOH, NHO(C₁-C₈)alkyl groups, —O-halogenated (C₁-C₈)alkyl groups (for example but not limited to —OCF₃), —S(O)₂-halogenated (C₁-C₈)alkyl groups (for example but not limited to —S(O)₂CF₃), —S-halogenated (C₁-C₈)alkyl groups (for example but not limited to —SCF₃), —(C₁-C₆) heterocycle (for example but not limited to pyrrolidine, tetrahydrofuran, pyran or morpholine), —(C₁-C₆) heteroaryl (for example but not limited to tetrazole, imidazole, furan, pyrazine or pyrazole), -phenyl, —NHC(O)O—(C₁-C₆)alkyl groups, —N((C₁-C₆)alkyl)C(O)O—(C₁-C₆)alkyl groups, —C(═NH)—(C₁-C₆)alkyl groups, —C(═NOH)—(C₁-C₆)alkyl groups, or —C(═N—O—(C₁-C₆)alkyl)-(C₁-C₆)alkyl groups.

Exemplary carbon atom substituents include, but are not limited to, halogen, —CN, —NO₂, —N₃, hydroxyl, alkoxy, cycloalkoxy, aryloxy, amino, monoalkyl amino, dialkyl amino, amide, sulfonamide, thiol, acyl, carboxylic acid, ester, sulfone, sulfoxide, alkyl, haloalkyl, alkenyl, alkynyl, C₃₋₁₀ carbocyclyl, C₆₋₁₀ aryl, 3-10 membered heterocyclyl, 5-10 membered heteroaryl, etc. For example, exemplary carbon atom substituents can include F, Cl, —CN, —SO₂H, —SO₃H, —OH, —OC₁₋₆ alkyl, —NH₂, —N(C₁₋₆ alkyl)₂, —NH(C₁₋₆ alkyl), —SH, —SC₁₋₆ alkyl, —C(═O)(C₁₋₆ alkyl), —CO₂H, —CO₂(C₁₋₄ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, C₁₋₆ alkyl, C₁₋₆ haloalkyl, C₂₋₆ alkenyl, C₂₋₆ alkynyl, C₃₋₁₀ carbocyclyl, C₆₋₁₀ to aryl, 3-10 membered heterocyclyl, 5-10 membered heteroaryl, or two geminal substituents can be joined to form ═O.

Nitrogen atoms can be substituted or unsubstituted as valency permits, and include primary, secondary, tertiary, and quaternary nitrogen atoms. Exemplary nitrogen atom substituents include, but are not limited to, hydrogen, acyl groups, esters, sulfone, sulfoxide, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two substituent groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl can be further substituted as defined herein. In embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, acyl groups, esters, sulfonates, C₁₋₁₀ alkyl, C₁₋₁₀ haloalkyl, C₂₋₁₀ to alkenyl, C₂₋₁₀ to alkynyl, C₃₋₁₀ to carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl can be further substituted as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3^{rd} edition, John Wiley & Sons, 1999, incorporated herein by reference. Exemplary oxygen protecting groups include, but are not limited to, those forming alkyl ethers or substituted alkyl ethers, such as methyl, allyl, benzyl, substituted benzyls such as 4-methoxybenzyl, methoxylmethyl (MOM), benzyloxymethyl (BOM), 2-methoxyethoxymethyl (MEM), etc., those forming silyl ethers, such as trimethylsilyl (TMS), triethylsilyl (TES), triisopropylsilyl (TIPS), t-butyldimethylsilyl (TBDMS), etc., those forming acetals or ketals, such as tetrahydropyranyl (THP), those forming esters such as formate, acetate, chloroacetate, dichloroacetate, trichloroacetate, trifluoroacetate, methoxyacetate, etc., those forming carbonates or sulfonates such as methanesulfonate (mesylate), benzylsulfonate, and tosylate (Ts), etc.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, the “optionally substituted” alkyl, alkenyl, alkynyl, carbocyclic, cycloalkyl, alkoxy, cycloalkoxy, or heterocyclic group herein can be unsubstituted or substituted with 1, 2, 3, or 4 substituents independently selected from F, Cl, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy, and fluoro-substituted C₁₋₄ alkoxy.

In embodiments, the “optionally substituted” aryl or heteroaryl group herein can be unsubstituted or substituted with 1, 2, 3, or 4 substituents independently selected from F, Cl, —OH, —CN, NH₂, protected amino, NH(C₁₋₄ alkyl) or a protected derivative thereof, N(C₁₋₄ alkyl((C₁₋₄ alkyl), —S(═O)(C₁₋₄ alkyl), —SO₂(C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC) and the formation and crystallization of chiral salts; or isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving. Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers including racemic mixtures.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

“Subject” refers to an animal, such as a mammal, that has been or will be the object of treatment, observation, or experiment. The methods described herein is useful for both human therapy and veterinary applications. In one embodiment, the subject is a human.

The term “treating” means one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “therapeutically effective” applied to dose or amount refers to that quantity of a compound or pharmaceutical formulation that is sufficient to result in a desired clinical benefit after administration to a patient in need thereof.

### Compounds

The compound of the present disclosure can be useful for modulating aldehyde dehydrogenase (e.g., ALDH1a3 and/or ALDH1a2). Further, the compounds of the present disclosure can be useful for treating various diseases and conditions including, but not limited to, cancers, cancer metastasis, and/or other ALDH1a2 and/or ALDH1a3-mediated diseases and disorders, such as type 2 diabetes, pulmonary arterial hypertension (PAH) and neointimal hyperplasia (NIH) or as a male contraceptive.

In embodiments, the present disclosure provides a compound of Formula (I):

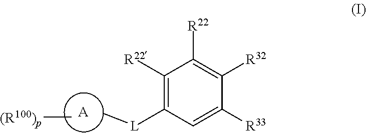

or a pharmaceutically acceptable salt thereof,
wherein:
   ring A is a heterocyclyl, heteroaryl, or aryl;
   L is —NH—, —C(O)—NH—, —C(O)—N(R^{A})-alkylene-O—, -(alkylene)-N(R^{A})-alkylene-O—, —S(O)NH—, —S(O)₂NH—, —S(O)—, or —S(O)₂—;
   R²² is halo, —CO—N(R^{A})₂, —CN, —C₁₋₆ alkyl, —C₁₋₆alkyl-CN, —C₁₋₆haloalkyl, or carbocyclyl;
   R²² is H, —CN, halo, —C₁₋₆ alkyl, or —C₁₋₆ haloalkyl; or
   R²² and R^{22′} are joined to form a heteroaryl, carbocyclyl, or heterocyclyl, each of which may be substituted with one or more halo;
   R³² and R³³ are joined to form a heterocyclyl and substituted with oxo; and wherein the heterocyclyl may be further optionally substituted with one or more R¹⁰¹;
   p is 0, 1 or 2;
   each R¹⁰⁰ is independently halo, —CN, hydroxy, —C₁₋₆ alkyl, —C₁₋₆ alkylene-carbocyclyl, —C₁₋₆ alkylene-heterocyclyl, —C₁₋₆ haloalkyl, carbocyclyl, aryl, or heteroaryl, wherein aryl or heteroaryl is optionally substituted with one or more R¹⁰¹;
   each R¹⁰¹ is independently halo, —CN, —CO—N(R^{A})₂, hydroxy, —C₁₋₆ alkyl, or —C₁₋₆haloalkyl; and
   R^{A} is H, —C₁₋₆ alkyl, or aryl.

In embodiments, the present disclosure provides a compound of Formula (I):

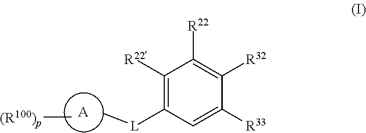

or a pharmaceutically acceptable salt thereof,
wherein:
   ring A is a heterocyclyl, heteroaryl, or aryl;
   L is —NH—, —C(O)—NH—, —C(O)—N(R^{A})-alkylene-O—, -(alkylene)-N(R^{A})-alkylene-O—, —S(O)NH—, —S(O)₂NH—, —S(O)—, or —S(O)₂—;
   R²² is halo, —CO—N(R^{A})₂, —CN, —C₁₋₆ alkyl, —C₁₋₆ alkyl-CN, —C₁₋₆ haloalkyl, or carbocyclyl;
   R^{22′} is H, —CN, halo, —C₁₋₆ alkyl, or —C₁₋₆ haloalkyl; or
   R²² and R^{22′} are joined to form a heteroaryl, carbocyclyl, or heterocyclyl, each of which may be substituted with one or more halo;
   R¹² and R³³ are joined to form a heterocyclyl and substituted with oxo; and wherein the heterocyclyl may be further optionally substituted with one or more R¹⁰¹;
   p is 0, 1, or 2;
   each R¹⁰⁰ is independently halo, —C₁₋₆ alkyl, —C₁₋₆ alkylene-carbocyclyl, —C₁₋₆ alkylene-heterocyclyl, —C₁₋₆ haloalkyl, or aryl, wherein aryl is optionally substituted with one or more R¹⁰¹;
   each R¹⁰¹ is independently halo, —CO—N(R^{A})₂, —CN, hydroxy, —C₁₋₆ alkyl, or —C₁₋₆ haloalkyl; and
   R^{A} is H, —C₁₋₆ alkyl, or aryl.

In embodiments of Formula (I) (and all other formulae disclosed herein), when L is —C(O)—NH— or —S(O)₂NH—, and A is aryl, then p is 1 or 2 and at least one 1 R¹⁰⁰ is aryl optionally substituted with one or more R¹⁰⁰. In embodiments of the compounds of Formula (I) (and all other formulae disclosed herein), when ring A is aryl, a 6-membered heteroaryl, a 6-membered heterocyclyl, a 5,6-fused bicyclic heteroaryl, or a 6,6-fused bicyclic heteroaryl, then p is 1 or 2 and at least 1 R¹⁰⁰ is aryl optionally substituted with 1 or 2 R¹⁰¹ In embodiments of Formula (I) (and all other formulae disclosed herein), the compound is not

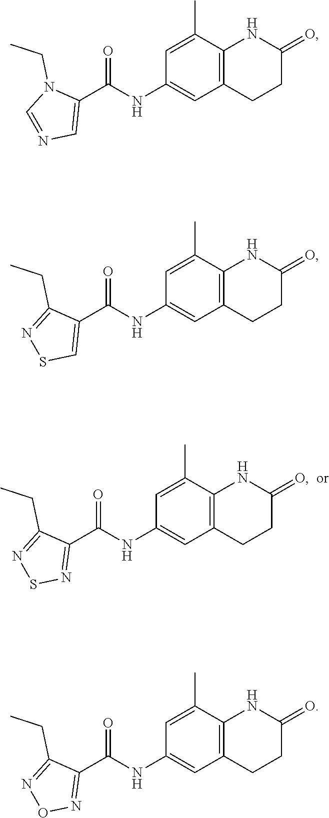

In embodiments, the compound of Formula (I) is a compound of Formula (II):

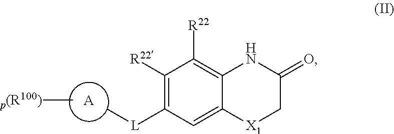

wherein:
   X₁ is —S—, —S(O)₂, —N(R^{A})—, or —CH₂—;
   ring A is a heterocyclyl, heteroaryl, or aryl;
   L is —NH—, —C(O)—NH—, —C(O)—N(R^{A})-alkylene-O—, -(alkylene)-N(R^{A})-alkylene-O—, —S(O)NH—, —S(O)₂NH—, —S(O)—, or —S(O)₂—;
   R²² is halo, —CO—N(R^{A})₂, —CN, —C₁₋₆ alkyl, —C₁₋₆ alkyl-CN, —C₁₋₆ haloalkyl, or carbocyclyl;
   R^{22′} is H, —CN, halo, —C₁₋₆ alkyl, or —C₁₋₆ haloalkyl; or
   R²² and R^{22′} are joined to form a heteroaryl, carbocyclyl, or heterocyclyl, each of which may be substituted with one or more halo;
   p is 0, 1, or 2;
   each R¹⁰⁰ is independently halo, —C₁₋₆ alkyl, —C₁₋₆ alkylene-carbocyclyl, —C₁₋₆ alkylene-heterocyclyl, —C₁₋₆ haloalkyl, or aryl, wherein aryl is optionally substituted with one or more R¹⁰¹;
   each R¹⁰¹ is independently halo, —CO—N(R^{A})₂, —CN, hydroxy, —C₁₋₆ alkyl, or —C₁₋₆ haloalkyl; and
   R^{A} is H, —C₁₋₆ alkyl, or aryl.

In embodiments, the compound of Formula (I) is a compound of Formula (II):

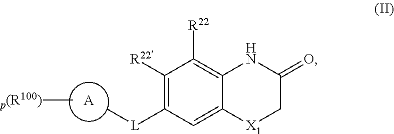

wherein:
   X₁ is —S—, —S(O)₂—, —N(R^{A})— or —CH₂—,
   ring A is heteroaryl or aryl;
   L is —NH—, —C(O)—NH—, —C(O)—N(R^{A})-alkylene-O—, or -(alkylene)-N(R^{A})-alkylene-O—;
   R²² is halo, —CN, —C₁₋₆ alkyl, —C₁₋₆ alkyl-CN, or —C₁₋₆ haloalkyl;
   R^{22′} is H;
   p is 0, 1, or 2;
   each R¹⁰⁰ is independently halo, —CN, hydroxy, —C₁₋₆ alkyl, —C₁₋₆ alkylene-carbocyclyl, —C₁₋₆alkylene-heterocyclyl, —C₁₋₆haloalkyl, carbocyclyl, aryl, or heteroaryl wherein aryl or heteroaryl is optionally substituted with one or more R¹⁰¹;
   each R¹⁰¹ is independently halo, —CN, hydroxy, or —C₁₋₆ alkyl; and
   R^{A} is H or —C₁₋₆ alkyl.

In embodiments, the compound of Formula (I) is a compound of Formula (II):

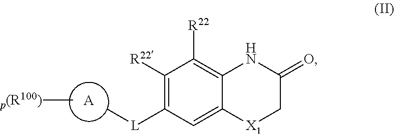

wherein:
   X₁ is —S—, —S(O)₂—, —N(R^{A})— or —CH₂—;
   ring A is heteroaryl or aryl;
   L is —NH—, —C(O)—NH—, —C(O)—N(R^{A})-alkylene-O—, or -(alkylene)-N(R^{A})-alkylene-O—;
   R²² is halo, —CN, —C₁₋₆ alkyl, —C₁₋₆ alkyl-CN, or —C₁₋₆ haloalkyl,
   R^{22′} is H;
   p is 0, 1, or 2;
   each R¹⁰⁰ is independently halo, —C₁₋₆ alkyl, —C₁₋₆ alkylene-carbocyclyl, —C₁₋₆ alkylene-heterocyclyl, —C₁₋₆ haloalkyl, or aryl, wherein aryl is optionally substituted with one or more R¹⁰¹;
   each R¹⁰¹ is independently halo, —CN, hydroxy, or —C₁₋₆ alkyl; and
   R^{A} is H or —C₁₋₆ alkyl.

In embodiments, the compound of Formula (I) is a compound of Formula (III):

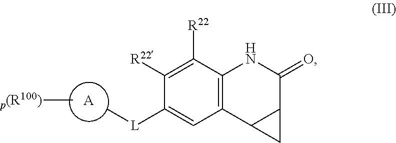

or a pharmaceutically acceptable salt or deuterated form thereof,
wherein:
   ring A is heteroaryl, heterocyclyl, or aryl;
   L is —C(O)—NH—, —C(NR^{A})—NH—, —C(O)—N(R^{A})-alkylene-O—, or -(alkylene)-N(R^{A})-alkylene-O—;
   R²² is halo, —CN, —C₁₋₆ alkyl, —C₁₋₆ alkyl-CN, —C₁₋₆ haloalkyl, -carbocyclyl, or —C₁₋₆ alkyl-carbocyclyl;
   R^{22′} is H, —CN, halo, —C₁₋₆ alkyl, —C₁₋₆ haloalkyl, —C₁₋₆alkyl-CN, carbocyclyl, or —C₁₋₆ alkyl-carbocyclyl;
   p is 0, 1, or 2;
   each R¹⁰⁰ is independently halo, —CN, hydroxy, —C₁₋₆ alkyl, —C₁₋₆ alkylene-carbocyclyl, —C₁₋₆ alkylene-heterocyclyl, —C₁₋₆ haloalkyl, carbocyclyl, aryl, or heteroaryl, wherein aryl or heteroaryl is optionally substituted with 1 or 2 R¹⁰¹;
   each R¹⁰¹ is independently halo, —CN, hydroxy, or —C₁₋₆ alkyl; and
   R^{A} is H or —C₁₋₆ alkyl
   wherein the compound is not

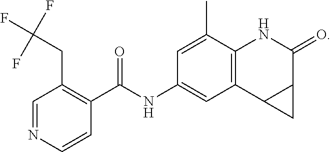 [SMILES: Cc1cc(NC(=O)c2ccncc2CC(F)(F)F)cc2c1NC(=O)C1CC21]

In embodiments, the compound of Formula (I) is a compound of Formula (IIA):

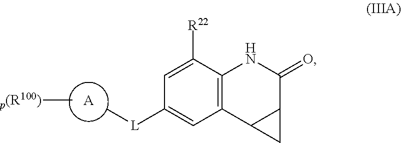

or a pharmaceutically acceptable salt or deuterated form thereof,
wherein:
   ring A is heteroaryl or aryl;
   L is —C(O)—NH—, —C(NR^{A})—NH—, —C(O)—N(R^{A})-alkylene-O—, or -(alkylene)-N(R^{A})-alkylene-O—;
   R²² is halo, —CN, —C₁₋₆ alkyl, —C₁₋₆ alkyl-CN, —C₁₋₆ haloalkyl, -carbocyclyl, or —C₁₋₆ alkyl-carbocyclyl;
   p is 0, 1, or 2;
   each R¹⁰⁰ is independently halo, —C₁₋₆ alkyl, —C₁₋₆ alkylene-carbocyclyl, —C₁₋₆ alkylene-heterocyclyl, —C₁₋₆ haloalkyl, or aryl, wherein aryl is optionally substituted with 1 or 2 R¹⁰¹;
   each R¹⁰¹ is independently halo, —CN, hydroxy, or —C₁₋₆ alkyl; and
   R^{A} is H or —C₁₋₆ alkyl
   wherein the compound is not

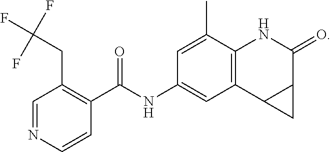 [SMILES: Cc1cc(NC(=O)c2ccncc2CC(F)(F)F)cc2c1NC(=O)C1CC21]

In embodiments, the compound of Formula (I) is a compound of Formula (IV):

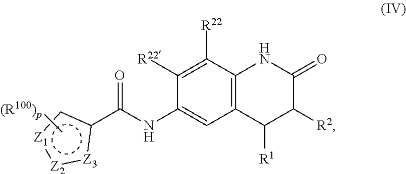

wherein:
R¹ is H and R² is H, or R and R² are joined to form a carbocyclyl;
R²² is halo, —CN, —C₁₋₆ alkyl, —C₁₋₆ alkyl-CN, —C₁₋₆ haloalkyl, carbocyclyl, or —C₁₋₆ alkyl-carbocyclyl;
p is 0, 1, or 2;
each R¹⁰⁰ is independently halo, —CN, hydroxy, —C₁₋₆alkylene-carbocyclyl, —C₁₋₆ alkyl, —C₁₋₆ haloalkyl, or carbocyclyl;
Z₁, Z₂, and Z₃ are independently selected from —CH—, —S—, —N—, —NH—, and —O—; and
dashed line represents a double or is absent to provide an aromatic ring;
wherein the compound is not

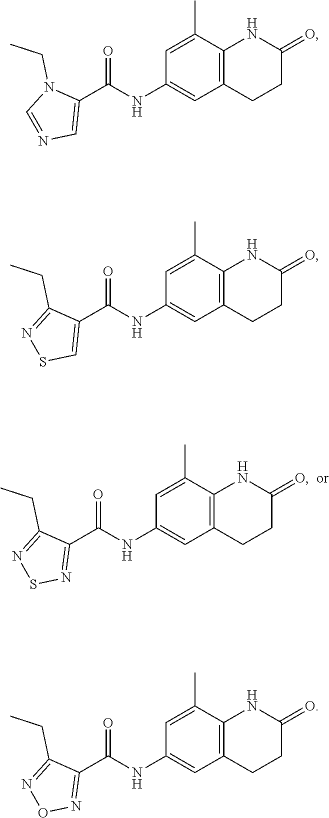

In embodiments, the compound of Formula (I) is a compound of Formula (IV):

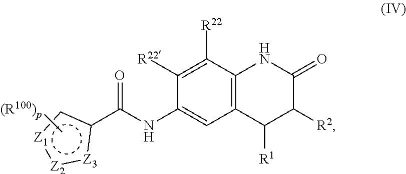

or a pharmaceutically acceptable salt or deuterated form thereof,
wherein:
R¹ is H and R² is H, or R and R² are joined to form a carbocyclyl;
R²² is halo, —CN, —C₁₋₆alkyl, —C₁₋₆alkyl-CN, —C₁₋₆haloalkyl, carbocyclyl, or —C₁₋₆ alkyl-carbocyclyl;
R^{22′} is H, —CN, halo, —C₁₋₆alkyl, —C₁₋₆haloalkyl, —C₁₋₆ alkyl-CN, carbocyclyl, or —C₁₋₆ alkyl-carbocyclyl;
p is 0, 1, or 2;
each R¹⁰⁰ is independently halo, —CN, hydroxy, —C₁₋₆alkylene-carbocyclyl, —C₁₋₆ alkyl, —C₁₋₆ haloalkyl, or carbocyclyl;
Z₁, Z₂, and Z₃ are independently selected from —CH—, —S—, —N—, —NH—, and —O—; and
dashed line represents a double or is absent to provide an aromatic ring;
wherein the compound is not

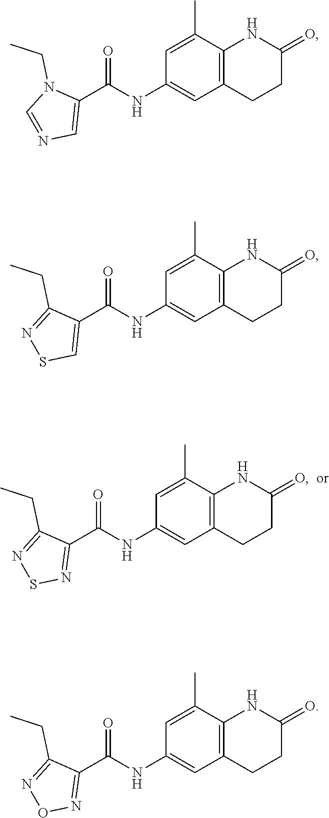

In embodiments, the compound of Formula (I) is a compound of Formula (IVA):

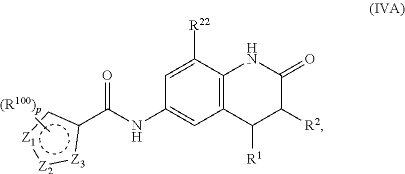

or a pharmaceutically acceptable salt or deuterated form thereof,
wherein:
R¹ is H and R² is H, or R¹ and R² are joined to form a carbocyclyl;
R²² is halo, —CN, —C₁₋₆alkyl, —C₁₋₆alkyl-CN, —C₁₋₆haloalkyl, carbocyclyl, or —C₁₋₆ alkyl-carbocyclyl;
p is 0, 1, or 2;
each R¹⁰⁰ is independently halo, —CN, hydroxy, —C₁₋₆ alkyl, or —C₁₋₆ haloalkyl;
Z₁, Z₂, and Z₃ are independently selected from —CH—, —S—, —N—, —NH—, and —O—; and
dashed line represents a double or is absent to provide an aromatic ring
wherein the compound is not

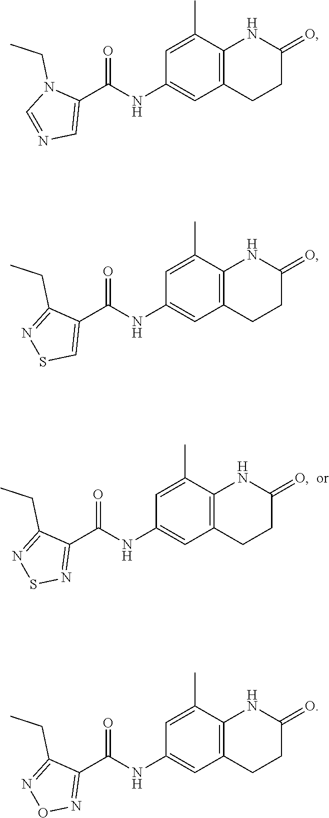

In embodiments, wherein the compound of Formula (I) is a compound of Formula (V):

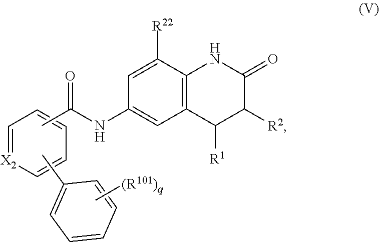

or a pharmaceutically acceptable salt or deuterated form thereof,
wherein:
R¹ is H and R² is H, or R¹ and R² are joined to form a carbocyclyl;
R²² is halo, —CN, —C₁₋₆ alkyl, —C₁₋₆ alkyl-CN, —C₁₋₆ haloalkyl, carbocyclyl or —C₁₋₆ alkyl-carbocyclyl;
q is 0, 1, or 2;
each R¹⁰¹ is independently halo, —CN, hydroxy, —C₁₋₆ alkyl, or —C₁₋₆ haloalkyl; and
X₂ is —CH— or —N—.

### L

In embodiments of the compound of Formula (I), (II), (III) or (IIIA), L is —NH—, —C(O)—NH—, —C(O)—N(R^{A})-alkylene-O—, -(alkylene)N(R^{A})-alkylene-O—, —S(O)NH—, —S(O)₂NH—, —S(O)—, or —S(O)₂—.

In embodiments of the compound of Formula (I), (II), (III) or (IIA), L is —C(O)—NH—, —C(O)—N(R^{A})-alkylene-O—, or -(alkylene)-N(R^{A})-alkylene-O—.

In embodiments of the compounds of Formula (I), (II), (III) or (IIIA), L is —C(O)—NH—.

In embodiments of the compounds of Formula (I), (II), (III) or (IIIA), L is —C(O)—N(R^{A})-alkylene-O—.

In embodiments of the compounds of Formula (I), (II), (III) or (IIIA), L is —C(O)—N(H)—CH₂CH₂—O— or —C(O)—N(CH₃)—CH₂CH₂—O—.

In embodiments of the compounds of Formula (I), (II), (III) or (IIA), L is -(alkylene)-N(R^{A})-alkylene-O—.

In embodiments of the compounds of Formula (I), (II), (III) or (IIIA), L is —CH₂—N(CH₃)—CH₂CH₂—O—.

### Ring A

In embodiments of the compounds of Formula (I), (II), (III) or (IIA), ring A is a heterocyclyl, heteroaryl, or aryl.

In embodiments of the compounds of Formula (I), (II), (III) or (IIIA), ring A is heteroaryl or aryl.

In embodiments of the compounds of Formula (I), (II), (III) or (IIIA), ring A is heteroaryl.

In embodiments of the compounds of Formula (I), (II), (III) or (IIIA), ring A is aryl.

In embodiments of the compounds of Formula (I), (II), (III) or (IIIA), ring A is a 5 or 6-membered heteroaryl, phenyl, a 5,6-bicyclic heteroaryl, a 5,6-bicyclic heterocyclyl, a 6,6-bicyclic heterocyclyl, a 6,6-bicyclic heteroaryl, or a 3-8 membered heterocyclyl.

In embodiments of the compounds of Formula (I), (II), (III) or (IIIA), ring A is pyridyl, phenyl, thiazolyl, isothiazolyl, imidazolyl, oxazolyl, thiadiazolyl, or isoxazolyl.

In embodiments of the compounds of Formula (I), (II), (III) or (IIIA), ring A is thiazolyl, isothiazolyl, imidazolyl, oxazolyl, thiadiazolyl, or isoxazolyl.

In embodiments of the compounds of Formula (I), (II), (III) or (IIIA) ring A is a 5 or 6-membered heteroaryl or phenyl.

In embodiments of the compounds of Formula (I), (II), (III) or (IIIA), ring A is a 5-membered heteroaryl or phenyl.

In embodiments of the compounds of Formula (I), (II), or (III), ring A is pyridyl, phenyl, thiazolyl, isothiazolyl, imidazolyl, oxazolyl, or thiadiazolyl.

In embodiments of the compounds of Formula (I), (II), (III) or (IIIA), ring A is phenyl, thiazolyl, isothiazolyl, imidazolyl, oxazolyl, or thiadiazolyl.

In embodiments of the compounds of Formula (I) (II), (III) or (IIIA), ring A is pyridyl. In embodiments, ring A is phenyl. In embodiments, ring A is thiazolyl. In embodiments, ring A is isothiazolyl. In embodiments, ring A is imidazolyl. In embodiments, ring A is oxazolyl. In embodiments, ring A is thiadiazolyl. In embodiments, ring A is isoxazolyl.

### R¹⁰⁰

In embodiments of the compounds of Formula (I), (II), (III), (IIIA), (IV) or (IVA), each R¹⁰⁰ is independently halo, —CN, hydroxy, —C₁₋₆ alkyl, —C₁₋₆ alkylene-carbocyclyl, —C₁₋₆ alkylene-heterocyclyl, —C₁₋₆ haloalkyl, carbocyclyl, aryl or heteroaryl, wherein aryl or heteroaryl is optionally substituted with 1 or 2 R¹⁰¹.

In embodiments of the compounds of Formula (I), (II), (IIIA), (IV) or (IVA), each R¹⁰⁰ is independently halo, —C₁₋₆ alkyl, —C₁₋₆ alkylene-carbocyclyl, —C₁₋₆ alkylene-heterocyclyl, —C₁₋₆ haloalkyl, or aryl, wherein aryl is optionally substituted with one or more R¹⁰¹.

In embodiments of the compounds of Formula (I), (II), (III), (IIIA), (IV) or (IVA), R¹⁰⁰ is independently halo, —CN, —C₁₋₆ haloalkyl, —C₁₋₆ alkylene-carbocyclyl, —C₁₋₆ alkyl, carbocyclyl, aryl, or heteroaryl, wherein aryl or heteroaryl is optionally substituted with one or more R¹⁰¹.

In embodiments of the compounds of Formula (I), (II), (IIIA), (IV) or (IVA), each R¹⁰⁰ is independently halo, —C₁₋₆ alkyl, —C₁₋₆ haloalkyl, or aryl, wherein aryl is optionally substituted with one or more R¹⁰¹.

In embodiments of the compounds of Formula (I), (II), (IIIA), (IV) or (IVA), each R¹⁰⁰ is halo. In embodiments, R¹⁰⁰ is —CN. In embodiments, R¹⁰⁰ is hydroxy. In embodiments, R¹⁰⁰ is —C₁₋₆ alkyl. In embodiments, R¹⁰⁰ is —C₁₋₆ alkylene-carbocyclyl e.g., —C₁₋₆ alkylene-cyclopropyl or in embodiments, —CH₂-cyclopropyl. In embodiments, R¹⁰⁰ is —C₁₋₆ alkylene-heterocyclyl. In embodiments, R¹⁰⁰ is —C₁₋₆ haloalkyl. In embodiments, R¹⁰⁰ is carbocyclyl e.g., cyclopropyl. In embodiments, R¹⁰⁰ is aryl wherein aryl is optionally substituted with 1 or 2 R¹⁰¹. In embodiments, R¹⁰⁰ is heteroaryl, wherein heteroaryl is optionally substituted with 1 or 2 R¹⁰¹.

In embodiments of the compounds of Formula (I), (II), (III), (IIIA), (IV) or (IVA), each R¹⁰⁰ is independently halo.

In embodiments of the compounds of Formula (I), (II), (III), (IIIA), (IV) or (IVA), each R¹⁰⁰ is —C₁₋₆ alkylene-cyclopropyl, —C₁₋₄ haloalkyl, or —C₁₋₆ alkyl. In embodiments, —C₁₋₆ alkylene-cyclopropyl is —CH₂-cyclopropyl. In embodiments, —C₁₋₆ alkyl is propyl or ethyl. In embodiments, —C₁₋₆ haloalkyl is —CH₂—CF₃.

In embodiments of the compounds of Formula (I), (II), (III), (IIIA), (IV) or (IVA), each R¹⁰⁰ is independently —C₁₋₆ alkyl. In embodiments, R¹⁰⁰ is —CH₂CH₃.

In embodiments of the compounds of Formula (I), (II), (III), (IIIA), (IV) or (IVA), each R¹⁰⁰ is independently —C₁₋₆ haloalkyl. In embodiments, R¹⁰⁰ is —CH₂CF₃.

In embodiments of the compounds of Formula (I), (II), (III), (IIIA), (IV) or (IVA), each R¹⁰⁰ is independently aryl, wherein aryl is optionally substituted with one or more R¹⁰¹.

Ring A-(R¹⁰⁰)ₚ

In embodiments of the compounds of Formula (I), (II), (III) or (IIIA), ring A-(R¹⁰⁰)ₚ is

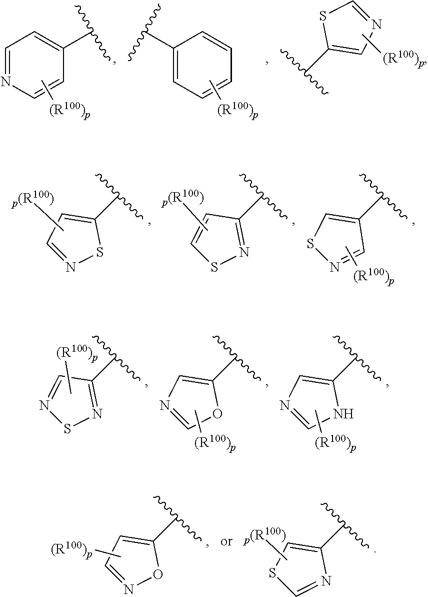

In embodiments of the compounds of Formula (I), (II), (III) or (IIA), ring A-(R¹⁰⁰)ₚ is

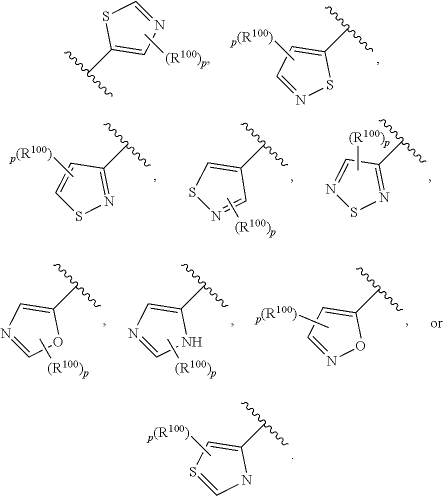

In embodiments of the compounds of Formula (I), (II), (III) or (IIIA), ring A-(R¹⁰⁰)ₚ is

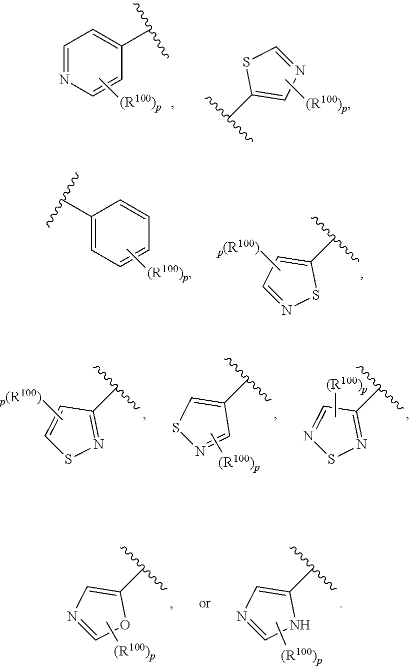

In embodiments of the compounds of Formula (I), (II), (III) or (IIIA), ring A-(R¹⁰⁰)ₚ is

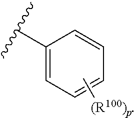

In embodiments, ring A-(R¹⁰⁰)ₚ is

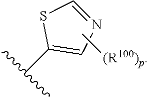

In embodiments, ring A-(R¹⁰⁰)ₚ is

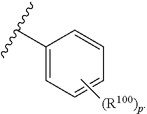

In embodiments, ring A-(R¹⁰⁰)ₚ is

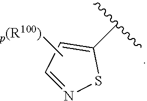

In embodiments, ring A-(R¹⁰⁰)ₚ is

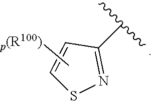

In embodiments, ring A-(R¹⁰⁰)ₚ is

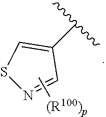

In embodiments, ring A-(R¹⁰⁰)ₚ is

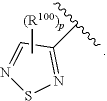

In embodiments ring A-(R¹⁰⁰)ₚ is

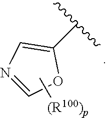

In embodiments ring A-(R¹⁰⁰)ₚ is

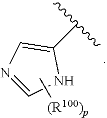

In embodiments ring A-(R¹⁰⁰)ₚ is

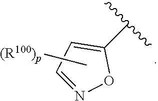

In embodiments ring A-(R¹⁰⁰)ₚ is

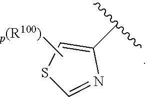

In embodiments of Formula (I), (II), (III) or (IIIA), ring A-(R¹⁰⁰)ₚ is:

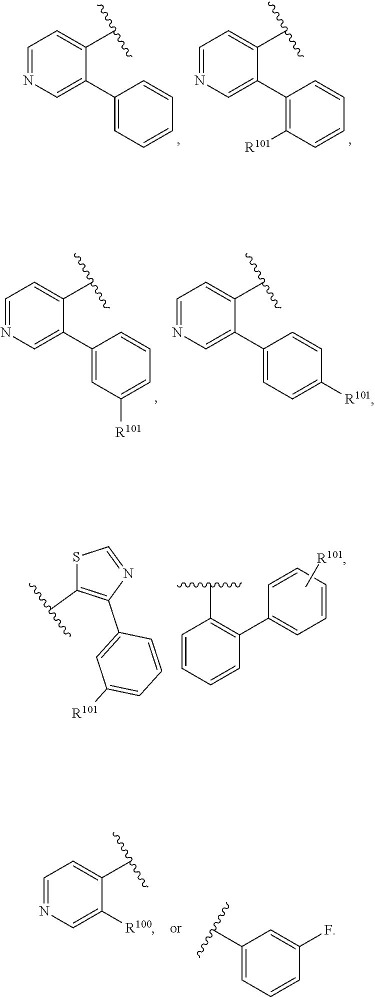

In embodiments of Formula (I), (II), (III) or (IIIA), ring A-(R¹⁰⁰)ₚ is:

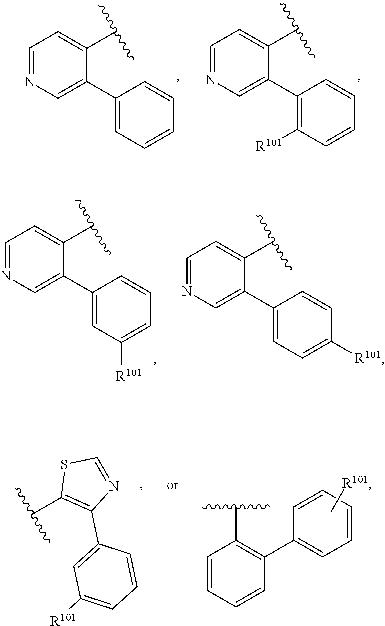

In embodiments of Formula (I), (II), (III) or (IIIA), ring A-(R¹⁰⁰)ₚ is:

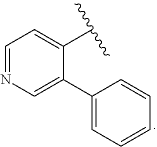 [SMILES: *c1ccncc1-c1ccccc1]

In embodiments, ring A-(R¹⁰⁰)ₚ is:

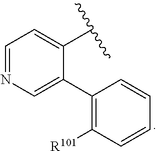

In embodiments, ring A-(R¹⁰⁰)ₚ is:

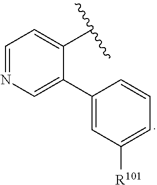

In embodiments, ring A-(R¹⁰⁰)ₚ is:

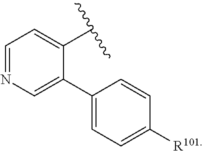

In embodiments, ring A-(R¹⁰⁰)ₚ is:

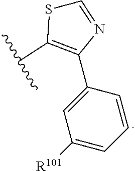

In embodiments, ring A-(R¹⁰⁰)ₚ is:

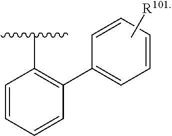

In embodiments, ring A-(R¹⁰⁰)ₚ is:

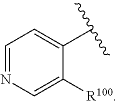

In embodiments, ring A-(R¹⁰⁰)ₚ is:

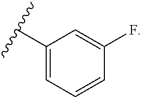 [SMILES: *c1cccc(F)c1]

In embodiments, of Formula (I), (II), (III) or (IIIA), ring A-(R¹⁰⁰)ₚ is:

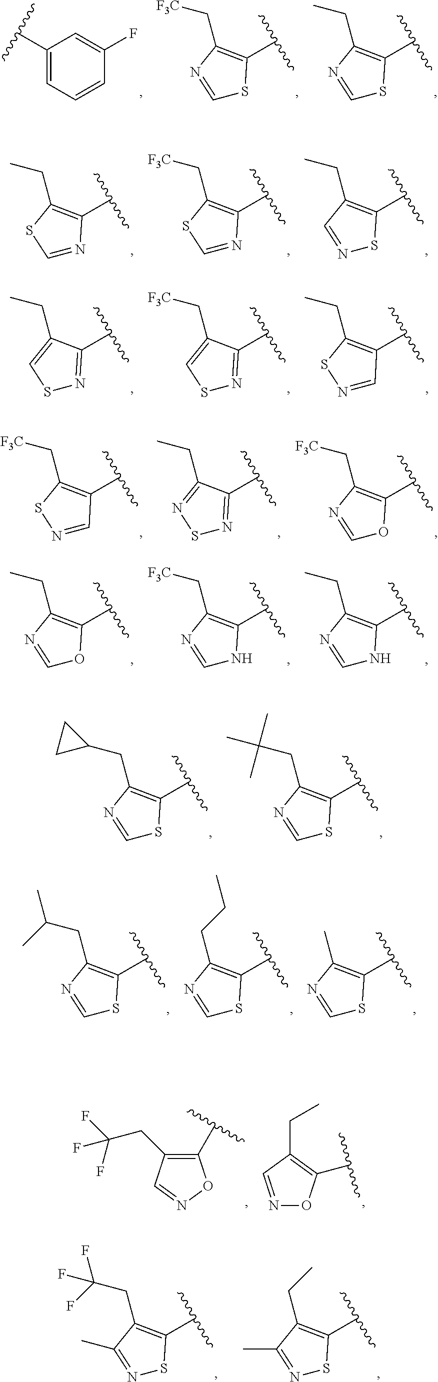

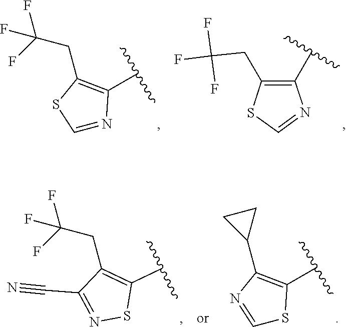

In embodiments, of Formula (I), (II), (III) or (IIIA), ring A-(R¹⁰⁰)ₚ is:

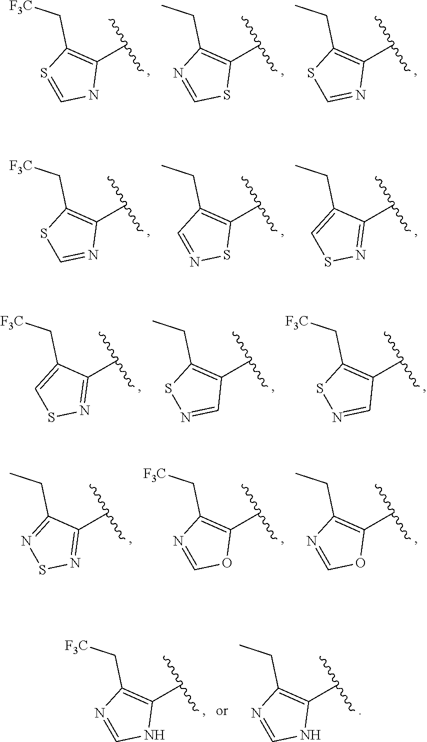

In embodiments, of Formula (I), (II), (III) or (IIIA), ring A-(R¹⁰⁰)ₚ is:

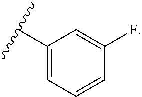 [SMILES: *c1cccc(F)c1]

In embodiments, ring A-(R¹⁰⁰)ₚ is:

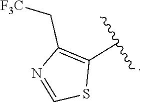 [SMILES: *c1scnc1CC(F)(F)F]

In embodiments, ring A-(R¹⁰⁰)ₚ is:

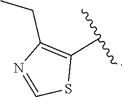 [SMILES: *c1scnc1CC]

In embodiments, ring A-(R¹⁰⁰)ₚ is:

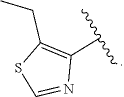 [SMILES: *c1ncsc1CC]

In embodiments, ring A-(R¹⁰⁰)ₚ is:

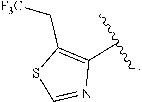 [SMILES: *c1ncsc1CC(F)(F)F]

In embodiments, ring A-(R¹⁰⁰)ₚ is:

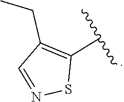 [SMILES: *c1sncc1CC]

In embodiments, ring A-(R¹⁰⁰)ₚ is:

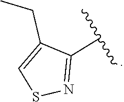 [SMILES: *c1nscc1CC]

In embodiments, ring A-(R¹⁰⁰)ₚ is:

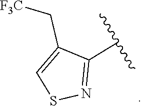 [SMILES: *c1nscc1CC(F)(F)F]

In embodiments, ring A-(R¹⁰⁰)ₚ is:

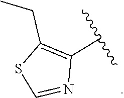 [SMILES: *c1ncsc1CC]

In embodiments, ring A-(R¹⁰⁰)ₚ is:

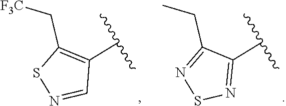

In embodiments, ring A-(R¹⁰⁰)ₚ is:

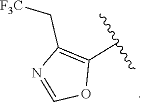 [SMILES: *c1ocnc1CC(F)(F)F]

In embodiments, ring A-(R¹⁰⁰)ₚ is:

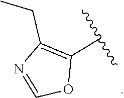 [SMILES: *c1ocnc1CC]

In embodiments, ring A-(R¹⁰⁰)ₚ is:

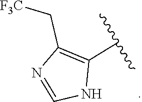 [SMILES: *c1[nH]cnc1CC(F)(F)F]

In embodiments, ring A-(R¹⁰⁰)ₚ is:

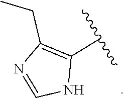 [SMILES: *c1[nH]cnc1CC]

In embodiments, ring A-(R¹⁰⁰)ₚ is:

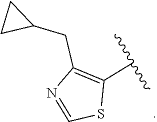 [SMILES: *c1scnc1CC1CC1]

In embodiments, ring A-(R¹⁰⁰)ₚ is:

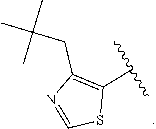 [SMILES: *c1scnc1CC(C)(C)C]

In embodiments, ring A-(R¹⁰⁰)ₚ is:

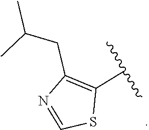 [SMILES: *c1scnc1CC(C)C]

In embodiments, ring A-(R¹⁰⁰)ₚ is:

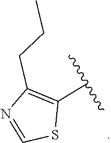 [SMILES: *c1scnc1CCC]

In embodiments, ring A-(R¹⁰⁰)ₚ is:

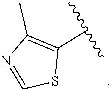 [SMILES: *c1scnc1C]

In embodiments, ring A-(R¹⁰⁰)ₚ is:

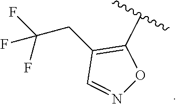 [SMILES: *c1oncc1CC(F)(F)F]

In embodiments, ring A-(R¹⁰⁰)ₚ is:

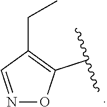 [SMILES: *c1oncc1CC]

In embodiments, ring A-(R¹⁰⁰)ₚ is:

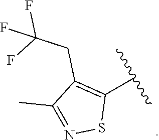 [SMILES: *c1snc(C)c1CC(F)(F)F]

In embodiments, ring A-(R¹⁰⁰)ₚ is:

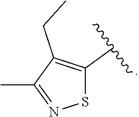 [SMILES: *c1snc(C)c1CC]

In embodiments, ring A-(R¹⁰⁰)ₚ is:

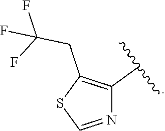 [SMILES: *c1ncsc1CC(F)(F)F]

In embodiments, ring A-(R¹⁰⁰)ₚ is:

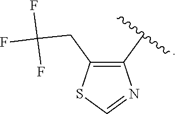 [SMILES: *c1ncsc1CC(F)(F)F]

In embodiments, ring A-(R¹⁰⁰)ₚ is:

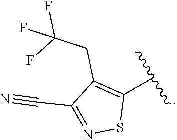 [SMILES: *c1snc(C#N)c1CC(F)(F)F]

In embodiments, ring A-(R¹⁰⁰)ₚ is:

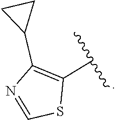 [SMILES: *c1scnc1C1CC1]

### R¹⁰¹

In embodiments of the compounds of Formula (I)—(V), each R¹⁰¹ is independently halo, —CO—N(R^{A})₂, —CN, —C₁₋₆ alkyl, —C₁₋₆ alkyl-CN, —C₁₋₆ haloalkyl, or carbocyclyl.

In embodiments of the compounds of Formula (I)—(V), each R¹⁰¹ is independently halo, —CN, hydroxy, —C₁₋₆ alkyl, or —C₁₋₆ haloalkyl.

In embodiments of the compounds of Formula (I)—(V), each R¹⁰¹ is independently halo, —CN, hydroxy, or —C₁₋₆ alkyl.

In embodiments of the compounds of Formula (I)—(V), each R¹⁰¹ is independently fluoro, hydroxy, —CN or methyl.

In embodiments of the compounds of Formula (I)—(V), each R¹⁰¹ is independently halo, e.g., fluoro.

In embodiments of the compounds of Formula (I)—(V), each R¹⁰¹ is —CN.

In embodiments of the compounds of Formula (I)—(V), each R¹⁰¹ is hydroxy.

In embodiments of the compounds of Formula (I), (II), (III), or (V), each R¹⁰¹ is independently —C₁₋₆ alkyl.

In embodiments of the compounds of Formula (I)—(V), each R¹⁰¹ is methyl.

R²² and R^{22′}

In embodiments of the compounds of Formula (I), (II), (III), (IIIA), (IV), (IVA) or (V), R²² is halo, —CO—N(R^{A})₂, —CN, —C₁₋₆ alkyl, —C₁₋₆ alkyl-CN, —C₁₋₆ haloalkyl, or carbocyclyl.

In embodiments of the compounds of Formula (I), (II), (II), (IIIA), (IV), (IVA) or (V), R²² is halo, —CN, —C₁₋₆ alkyl, —C₁₋₆ alkyl-CN, —C₁₋₆ haloalkyl, or carbocyclyl.

In embodiments of the compounds of Formula (I), (II), (IIIA), (IV), (TVA) or (V), R²² is halo, —CN, —C₁₋₆ alkyl, —C₁₋₆ alkyl-CN, or —C₁₋₆ haloalkyl.

In embodiments of the compounds of Formula (I), (II), (IIIA), (IV), (IVA) or (V), R²² is halo, —CN, —C₁₋₆ alkyl, or C₁₋₆ haloalkyl. In embodiments of the compounds of Formula (I) or (II), R²² is halo or —C₁₋₆ alkyl.

In embodiments of the compounds of Formula (I), (II), (IIIA), (IV), (IVA) or (V), R²² is methyl or chloro.

In embodiments of the compounds of Formula (I), (II), (IIIA), (IV), (IVA) or (V), R²² is methyl.

In embodiments of the compounds of Formula (I), (II), (IIIA), (IV), (IVA) or (V), R²² is chloro.

In embodiments of the compounds of Formula (I), (III), or (IV), R²² is H, —CN, halo, —C₁₋₆ alkyl, or —C₁₋₆ haloalkyl.

In embodiments of the compounds of Formula (I), (III), or (IV), R²² is H, halo, —C₁₋₆ alkyl, or —C₁₋₆ haloalkyl.

In embodiments of the compounds of Formula (I), (III), or (IV), R^{22′} is —H, halo, —C₁₋₆ alkyl, or —C₁₋₆ haloalkyl.

In embodiments of the compounds of Formula (I), (III), or (IV), R²² is —H, —F, or —Cl.

In embodiments of the compounds of Formula (I), (III), or (IV), R²² is —H or F.

In embodiments of the compounds of Formula (I), (III), or (IV), R²² is —H.

In embodiments of the compounds of Formula (I), (III), or (IV), R²² and R^{22′} are joined to form a heteroaryl, carbocyclyl, or heterocyclyl, each of which may be substituted with one or more halo.

R³²³ and R³³

In embodiments of the compounds of Formula (I), R³² and R³³ are joined to form a heterocyclyl and substituted with oxo; and wherein the heterocyclyl may be further optionally substituted with one or more R¹⁰¹.

In embodiments of the compounds of Formula (I), R³² and R³³ are joined to form a 6 or 7-membered heterocyclyl and substituted with oxo; and wherein the heterocyclyl may be further optionally substituted with one or more R¹⁰¹.

In embodiments of the compounds of Formula (I), R³² and R³³ are joined to form a 6 or 7-membered heterocyclyl containing one or two heteroatoms independently selected from O, or NR^{A}, and S and wherein the heterocyclyl is substituted with oxo; and wherein the heterocyclyl may be further optionally substituted with one or more R¹⁰¹.

In embodiments of the compounds of Formula (I), R³² and R³³ are joined to form a 6 or 7-membered heterocyclyl containing one or two heteroatoms independently selected from NR^{A} and S and wherein the heterocyclyl is substituted with oxo; and wherein the heterocyclyl may be further optionally substituted with one or more R¹⁰¹.

In embodiments of the compounds of Formula (I), R³² and R³³ are joined to form a 6 or 7-membered heterocyclyl containing one or two heteroatoms independently selected from NH and S and wherein the heterocyclyl is substituted with oxo; and wherein the heterocyclyl may be further optionally substituted with one or more R¹⁰¹.

### Integer p

In embodiments of the compounds of Formula (I), (II), (III), or (IV), p is 0, 1, or 2. In embodiments of the compounds of Formula (I), (II), (III), or (IV) p is 0 or 1. In embodiments of the compounds of Formula (I), (II), (Ill), or (IV), p is 0. In embodiments of the compounds of Formula (I), (II), (III), or (IV), p is 1. In embodiments of the compounds of Formula (I), (II), (III), or (IV), p is 2.

In embodiments of the compounds of Formula (I), (II), (III) or (IV), R^{A} is H, —C₁₋₆ alkyl, or aryl.

In embodiments of the compounds of Formula (I), (II), (III) or (IV), R^{A} is H or —C₁₋₆ alkyl.

In embodiments of the compounds of Formula (I), (II), (III) or (IV), R^{A} is H.

In embodiments of the compounds of Formula (I), (II), (III) or (IV), R^{A} is —C₁₋₆alkyl.

In embodiments of the compounds of Formula (I), (II), (III) or (IV), R^{A} is —C₁₋₃ alkyl.

In embodiments of the compounds of Formula (I), (II), (III) or (IV), R^{A} is methyl.

### X₁

In embodiments of the compounds of Formula (II), X₁ is —S—, —S(O)₂—, or —CH₂—.

In embodiments of the compounds of Formula (II), X₁ is —S— or —CH₂—.

In embodiments of the compounds of Formula (II), X₁ is —S—.

In embodiments of the compounds of Formula (II), X₁ is —CH₂—.

In embodiments of the compounds of Formula (II), Xₜ is S(O)₂.

### X₂

In embodiments of Formula (V), X₂ is —CH— or —N—. In embodiments of Formula (V), X₂ is —CH—. In embodiments of Formula (V), X₂ is —N—.

R¹ and R²

In embodiments of Formula (IV) or (V), R¹ is H and R² is H, or R¹ and R² are joined to form a carbocyclyl. In embodiments of Formula (IV) or (V), R¹ is H and R² is H. In embodiments of Formula (IV) or (V), R¹ and R² are joined to form a carbocyclyl. In embodiments, the carbocyclyl is a 3-6 membered carbocyclyl. In embodiments, the 3-6 membered carbocyclyl is cyclopropyl.

### Integer q

In embodiments of the compounds of Formula (V), q is 0, 1 or 2. In embodiments of the compounds of Formula (V), q is 0 or 1. In embodiments of the compounds of Formula (V), q is 0. In embodiments of the compounds of Formula (V), q is 1. In embodiments of the compounds of Formula (V), q is 2.

In embodiments, provided herein is one or more compounds in Table 1.

In embodiments, provided herein is a pharmaceutically acceptable salt of one or more compounds in Table 1.

TABLE 1
Compounds

| No. | Compound |
|---|---|
| 1 | 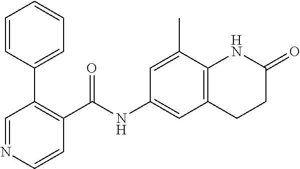 [SMILES: Cc1cc(NC(=O)c2ccncc2-c2ccccc2)cc2c1NC(=O)CC2] |
| 2 | 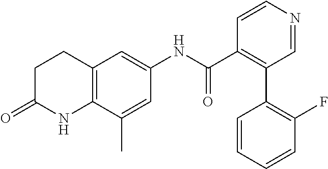 [SMILES: Cc1cc(NC(=O)c2ccncc2-c2ccccc2F)cc2c1NC(=O)CC2] |
| 3 | 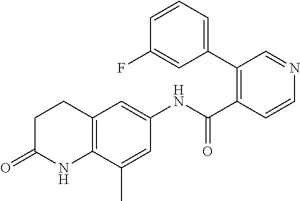 [SMILES: Cc1cc(NC(=O)c2ccncc2-c2cccc(F)c2)cc2c1NC(=O)CC2] |
| 4 | 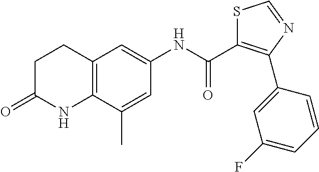 [SMILES: Cc1cc(NC(=O)c2scnc2-c2cccc(F)c2)cc2c1NC(=O)CC2] |
| 5 | 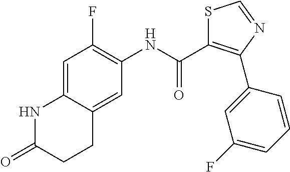 [SMILES: O=C1CCc2cc(NC(=O)c3scnc3-c3cccc(F)c3)c(F)cc2N1] |
| 6 | 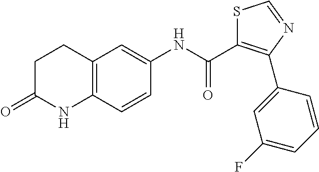 [SMILES: O=C1CCc2cc(NC(=O)c3scnc3-c3cccc(F)c3)ccc2N1] |
| 7 | 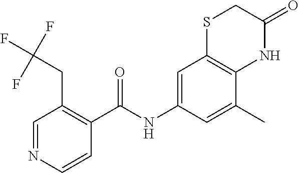 [SMILES: Cc1cc(NC(=O)c2ccncc2CC(F)(F)F)cc2c1NC(=O)CS2] |
| 8 | 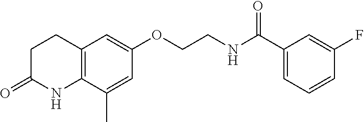 [SMILES: Cc1cc(OCCNC(=O)c2cccc(F)c2)cc2c1NC(=O)CC2] |
| 9 | 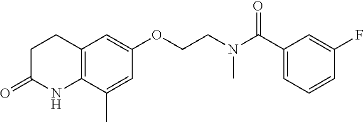 [SMILES: Cc1cc(OCCN(C)C(=O)c2cccc(F)c2)cc2c1NC(=O)CC2] |
| 10 | 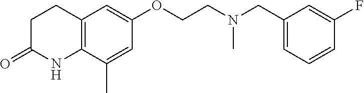 [SMILES: Cc1cc(OCCN(C)Cc2cccc(F)c2)cc2c1NC(=O)CC2] |
| 11 | 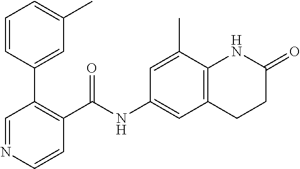 [SMILES: Cc1cccc(-c2cnccc2C(=O)Nc2cc(C)c3c(c2)CCC(=O)N3)c1] |
| 12 | 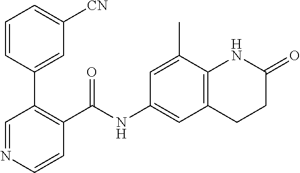 [SMILES: Cc1cc(NC(=O)c2ccncc2-c2cccc(C#N)c2)cc2c1NC(=O)CC2] |
| 13 | 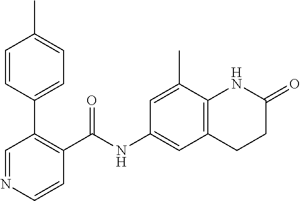 [SMILES: Cc1ccc(-c2cnccc2C(=O)Nc2cc(C)c3c(c2)CCC(=O)N3)cc1] |
| 14 | 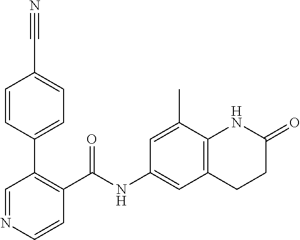 [SMILES: Cc1cc(NC(=O)c2ccncc2-c2ccc(C#N)cc2)cc2c1NC(=O)CC2] |
| 15 | 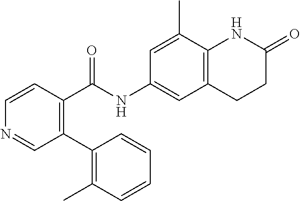 [SMILES: Cc1ccccc1-c1cnccc1C(=O)Nc1cc(C)c2c(c1)CCC(=O)N2] |
| 16 | 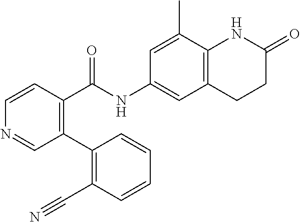 [SMILES: Cc1cc(NC(=O)c2ccncc2-c2ccccc2C#N)cc2c1NC(=O)CC2] |
| 17 | 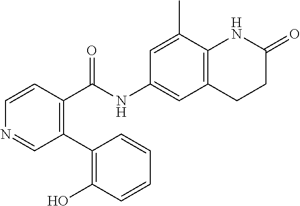 [SMILES: Cc1cc(NC(=O)c2ccncc2-c2ccccc2O)cc2c1NC(=O)CC2] |
| 18 | 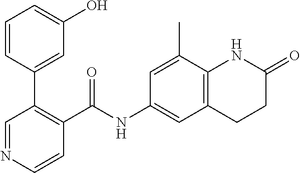 [SMILES: Cc1cc(NC(=O)c2ccncc2-c2cccc(O)c2)cc2c1NC(=O)CC2] |
| 19 | 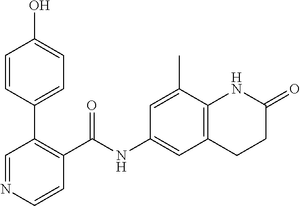 [SMILES: Cc1cc(NC(=O)c2ccncc2-c2ccc(O)cc2)cc2c1NC(=O)CC2] |
| 20 | 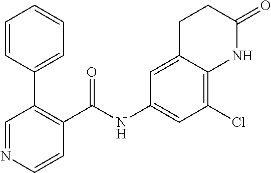 [SMILES: O=C1CCc2cc(NC(=O)c3ccncc3-c3ccccc3)cc(Cl)c2N1] |
| 21 | 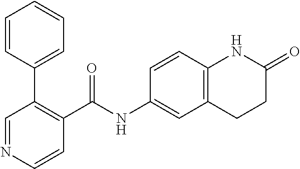 [SMILES: O=C1CCc2cc(NC(=O)c3ccncc3-c3ccccc3)ccc2N1] |
| 22 | 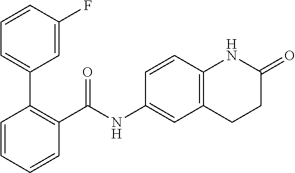 [SMILES: O=C1CCc2cc(NC(=O)c3ccccc3-c3cccc(F)c3)ccc2N1] |
| 23 | 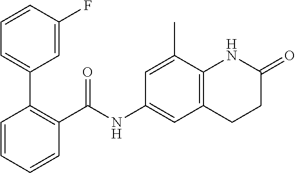 [SMILES: Cc1cc(NC(=O)c2ccccc2-c2cccc(F)c2)cc2c1NC(=O)CC2] |
| 24 | 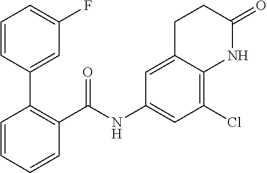 [SMILES: O=C1CCc2cc(NC(=O)c3ccccc3-c3cccc(F)c3)cc(Cl)c2N1] |
| 25 | 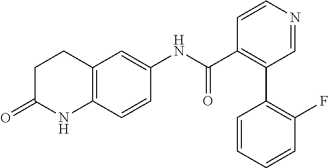 [SMILES: O=C1CCc2cc(NC(=O)c3ccncc3-c3ccccc3F)ccc2N1] |
| 26 | 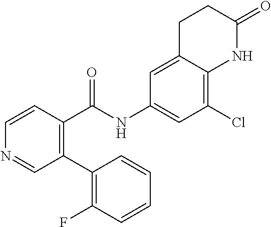 [SMILES: O=C1CCc2cc(NC(=O)c3ccncc3-c3ccccc3F)cc(Cl)c2N1] |
| 27 | 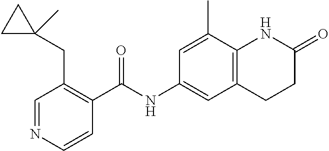 [SMILES: Cc1cc(NC(=O)c2ccncc2CC2(C)CC2)cc2c1NC(=O)CC2] |
| 28 | 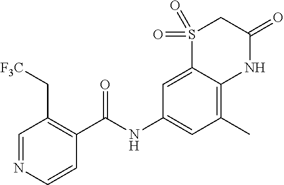 [SMILES: Cc1cc(NC(=O)c2ccncc2CC(F)(F)F)cc2c1NC(=O)CS2(=O)=O] |
| 29 | 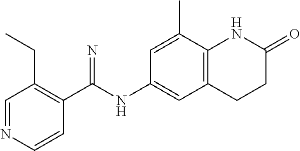 [SMILES: CCc1cnccc1C(=N)Nc1cc(C)c2c(c1)CCC(=O)N2] |
| 30 | 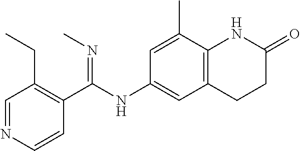 [SMILES: CCc1cnccc1/C(=N\C)Nc1cc(C)c2c(c1)CCC(=O)N2] |
| 31 | 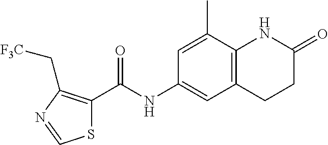 [SMILES: Cc1cc(NC(=O)c2scnc2CC(F)(F)F)cc2c1NC(=O)CC2] |
| 32 | 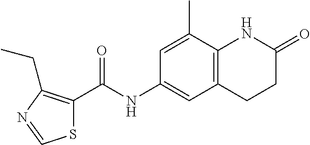 [SMILES: CCc1ncsc1C(=O)Nc1cc(C)c2c(c1)CCC(=O)N2] |
| 33 | 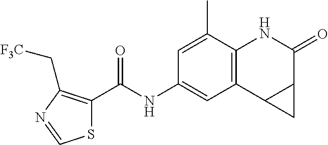 [SMILES: Cc1cc(NC(=O)c2scnc2CC(F)(F)F)cc2c1NC(=O)C1CC21] |
| 34 | 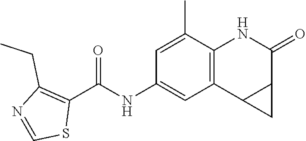 [SMILES: CCc1ncsc1C(=O)Nc1cc(C)c2c(c1)C1CC1C(=O)N2] |
| 35 | 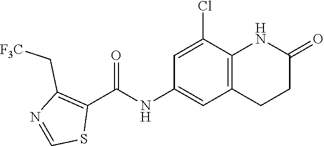 [SMILES: O=C1CCc2cc(NC(=O)c3scnc3CC(F)(F)F)cc(Cl)c2N1] |
| 36 | 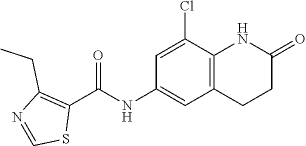 [SMILES: CCc1ncsc1C(=O)Nc1cc(Cl)c2c(c1)CCC(=O)N2] |
| 37 | 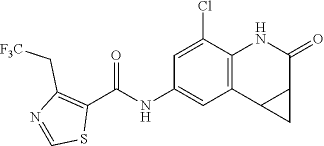 [SMILES: O=C(Nc1cc(Cl)c2c(c1)C1CC1C(=O)N2)c1scnc1CC(F)(F)F] |
| 38 | 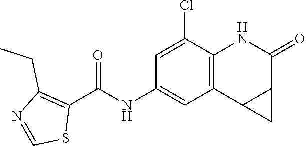 [SMILES: CCc1ncsc1C(=O)Nc1cc(Cl)c2c(c1)C1CC1C(=O)N2] |
| 39 | 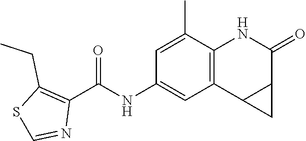 [SMILES: CCc1scnc1C(=O)Nc1cc(C)c2c(c1)C1CC1C(=O)N2] |
| 40 | 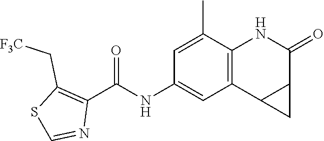 [SMILES: Cc1cc(NC(=O)c2ncsc2CC(F)(F)F)cc2c1NC(=O)C1CC21] |
| 41 | 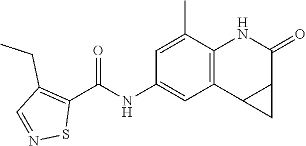 [SMILES: CCc1cnsc1C(=O)Nc1cc(C)c2c(c1)C1CC1C(=O)N2] |
| 42 | 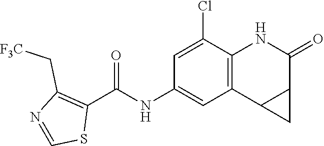 [SMILES: O=C(Nc1cc(Cl)c2c(c1)C1CC1C(=O)N2)c1scnc1CC(F)(F)F] |
| 43 | 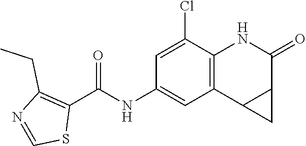 [SMILES: CCc1ncsc1C(=O)Nc1cc(Cl)c2c(c1)C1CC1C(=O)N2] |
| 44 | 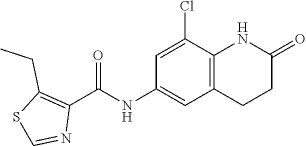 [SMILES: CCc1scnc1C(=O)Nc1cc(Cl)c2c(c1)CCC(=O)N2] |
| 45 | 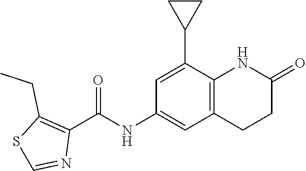 [SMILES: CCc1scnc1C(=O)Nc1cc2c(c(C3CC3)c1)NC(=O)CC2] |
| 46 | 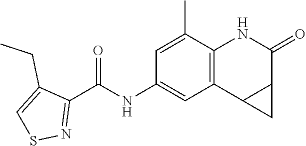 [SMILES: CCc1csnc1C(=O)Nc1cc(C)c2c(c1)C1CC1C(=O)N2] |
| 47 | 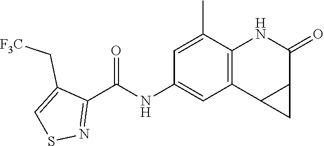 [SMILES: Cc1cc(NC(=O)c2nscc2CC(F)(F)F)cc2c1NC(=O)C1CC21] |
| 48 | 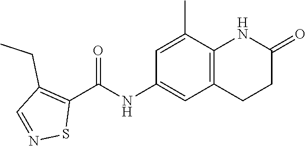 [SMILES: CCc1cnsc1C(=O)Nc1cc(C)c2c(c1)CCC(=O)N2] |
| 49 | 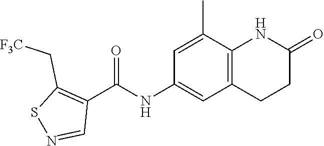 [SMILES: Cc1cc(NC(=O)c2cnsc2CC(F)(F)F)cc2c1NC(=O)CC2] |
| 50 | 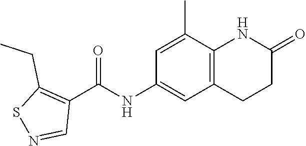 [SMILES: CCc1sncc1C(=O)Nc1cc(C)c2c(c1)CCC(=O)N2] |
| 51 | 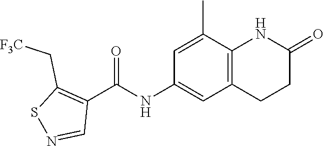 [SMILES: Cc1cc(NC(=O)c2cnsc2CC(F)(F)F)cc2c1NC(=O)CC2] |
| 52 | 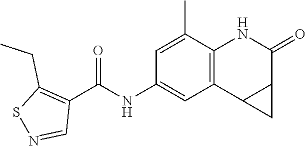 [SMILES: CCc1sncc1C(=O)Nc1cc(C)c2c(c1)C1CC1C(=O)N2] |
| 53 | 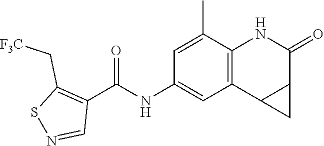 [SMILES: Cc1cc(NC(=O)c2cnsc2CC(F)(F)F)cc2c1NC(=O)C1CC21] |
| 54 | 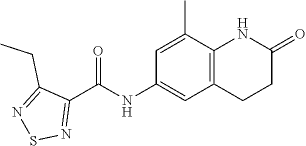 [SMILES: CCc1nsnc1C(=O)Nc1cc(C)c2c(c1)CCC(=O)N2] |
| 55 | 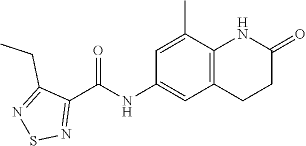 [SMILES: CCc1nsnc1C(=O)Nc1cc(C)c2c(c1)CCC(=O)N2] |
| 56 | 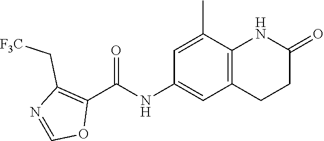 [SMILES: Cc1cc(NC(=O)c2ocnc2CC(F)(F)F)cc2c1NC(=O)CC2] |
| 57 | 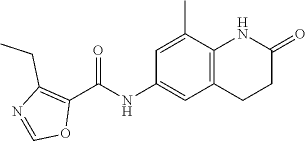 [SMILES: CCc1ncoc1C(=O)Nc1cc(C)c2c(c1)CCC(=O)N2] |
| 58 | 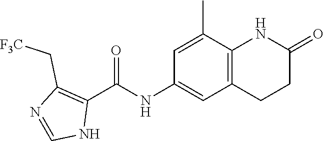 [SMILES: Cc1cc(NC(=O)c2[nH]cnc2CC(F)(F)F)cc2c1NC(=O)CC2] |
| 59 | 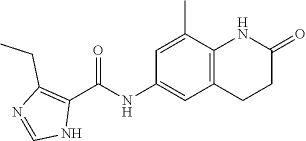 [SMILES: CCc1nc[nH]c1C(=O)Nc1cc(C)c2c(c1)CCC(=O)N2] |
| 60 | 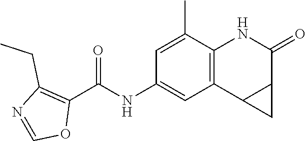 [SMILES: CCc1ncoc1C(=O)Nc1cc(C)c2c(c1)C1CC1C(=O)N2] |
| 61 | 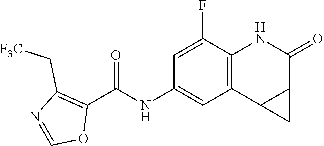 [SMILES: O=C(Nc1cc(F)c2c(c1)C1CC1C(=O)N2)c1ocnc1CC(F)(F)F] |
| 62 | 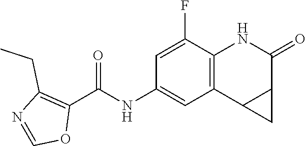 [SMILES: CCc1ncoc1C(=O)Nc1cc(F)c2c(c1)C1CC1C(=O)N2] |
| 63 | 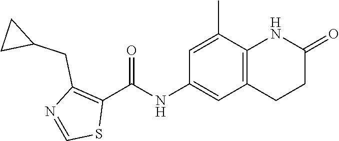 [SMILES: Cc1cc(NC(=O)c2scnc2CC2CC2)cc2c1NC(=O)CC2] |
| 64 | 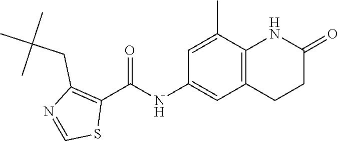 [SMILES: Cc1cc(NC(=O)c2scnc2CC(C)(C)C)cc2c1NC(=O)CC2] |
| 65 | 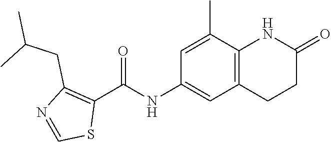 [SMILES: Cc1cc(NC(=O)c2scnc2CC(C)C)cc2c1NC(=O)CC2] |
| 66 | 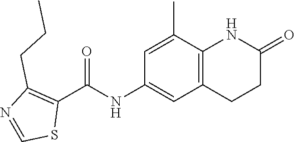 [SMILES: CCCc1ncsc1C(=O)Nc1cc(C)c2c(c1)CCC(=O)N2] |
| 67 | 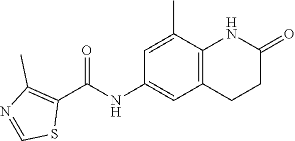 [SMILES: Cc1cc(NC(=O)c2scnc2C)cc2c1NC(=O)CC2] |
| 68 | 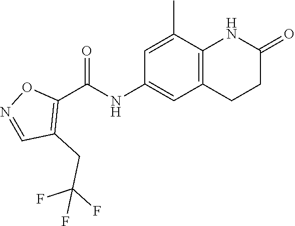 [SMILES: Cc1cc(NC(=O)c2oncc2CC(F)(F)F)cc2c1NC(=O)CC2] |
| 69 | 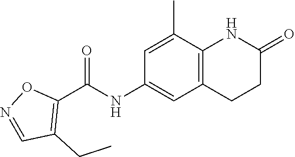 [SMILES: CCc1cnoc1C(=O)Nc1cc(C)c2c(c1)CCC(=O)N2] |
| 70 | 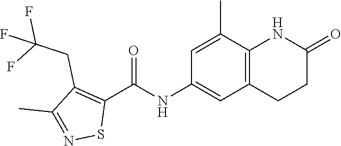 [SMILES: Cc1cc(NC(=O)c2snc(C)c2CC(F)(F)F)cc2c1NC(=O)CC2] |
| 71 | 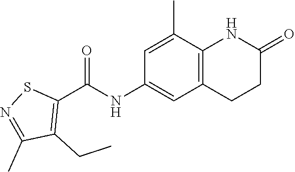 [SMILES: CCc1c(C)nsc1C(=O)Nc1cc(C)c2c(c1)CCC(=O)N2] |
| 72 | 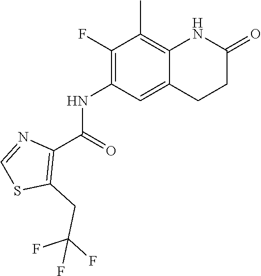 [SMILES: Cc1c(F)c(NC(=O)c2ncsc2CC(F)(F)F)cc2c1NC(=O)CC2] |
| 73 | 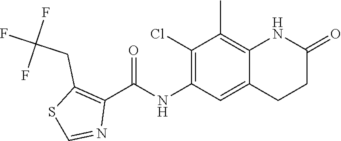 [SMILES: Cc1c(Cl)c(NC(=O)c2ncsc2CC(F)(F)F)cc2c1NC(=O)CC2] |
| 74 | 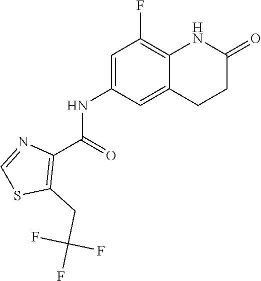 [SMILES: O=C1CCc2cc(NC(=O)c3ncsc3CC(F)(F)F)cc(F)c2N1] |
| 75 | 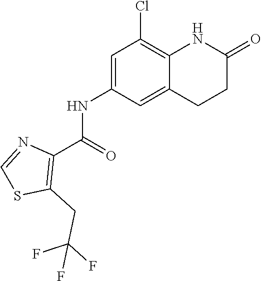 [SMILES: O=C1CCc2cc(NC(=O)c3ncsc3CC(F)(F)F)cc(Cl)c2N1] |
| 76 | 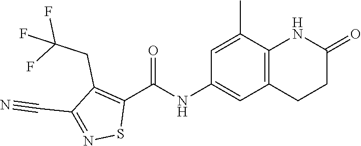 [SMILES: Cc1cc(NC(=O)c2snc(C#N)c2CC(F)(F)F)cc2c1NC(=O)CC2] |
| 77 | 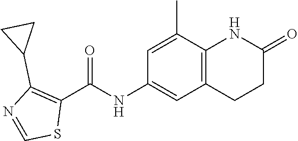 [SMILES: Cc1cc(NC(=O)c2scnc2C2CC2)cc2c1NC(=O)CC2] |
| 78 | 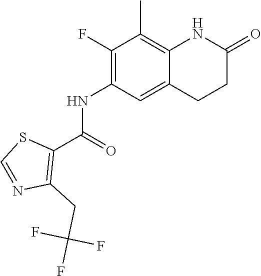 [SMILES: Cc1c(F)c(NC(=O)c2scnc2CC(F)(F)F)cc2c1NC(=O)CC2] |
| 79 | 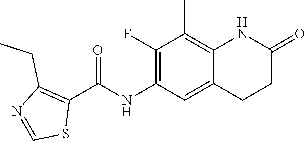 [SMILES: CCc1ncsc1C(=O)Nc1cc2c(c(C)c1F)NC(=O)CC2] |
| 80 | 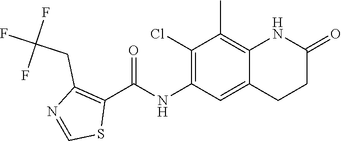 [SMILES: Cc1c(Cl)c(NC(=O)c2scnc2CC(F)(F)F)cc2c1NC(=O)CC2] |
| 81 | 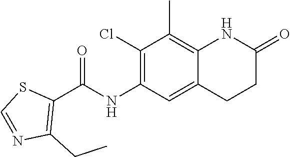 [SMILES: CCc1ncsc1C(=O)Nc1cc2c(c(C)c1Cl)NC(=O)CC2] |
| 82 | 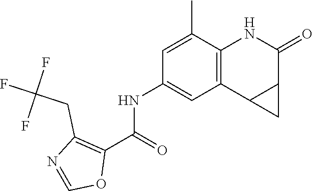 [SMILES: Cc1cc(NC(=O)c2ocnc2CC(F)(F)F)cc2c1NC(=O)C1CC21] |
| 83 | 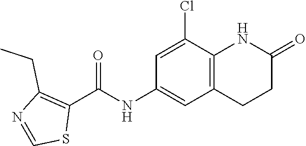 [SMILES: CCc1ncsc1C(=O)Nc1cc(Cl)c2c(c1)CCC(=O)N2] |

### Compositions

In embodiments, the present disclosure provides pharmaceutical compositions for modulating (e.g., inhibiting) aldehyde dehydrogenase (e.g., ALDH1a3 and/or ALDH1a2) in a subject. In some embodiments, a pharmaceutical composition comprises one or more compounds of the present disclosure (e.g., a compound of Formula (I), (II), (III), (IV), (V), or Table 1) or a pharmaceutically acceptable salt thereof.

In embodiments of the present disclosure, a pharmaceutical composition comprises a therapeutically effective amounts of one or more compounds of the present disclosure (e.g., a compound of Formula (I), (II), (III), (IV), (V), or Table 1) or a pharmaceutically acceptable salt thereof and a pharmaceutically acceptable carrier.

In embodiments, a pharmaceutical composition, as described herein, comprises one or more compounds selected from Table 1, or a pharmaceutically acceptable salt thereof and a pharmaceutically acceptable carrier.

In embodiments of the present disclosure, a pharmaceutical composition comprising one or more compounds of the present disclosure (e.g., a compound of Formula (I), (II), (III), (IV), (V), or Table 1) or a pharmaceutically acceptable salt thereof, and a pharmaceutically acceptable excipient or adjuvant is provided. The pharmaceutically acceptable excipients and adjuvants are added to the composition or formulation for a variety of purposes. In some embodiments, a pharmaceutical composition comprising one or more compounds disclosed herein, or a pharmaceutically acceptable salt thereof, further comprise a pharmaceutically acceptable carrier.

In some embodiments, a pharmaceutically acceptable carrier includes a pharmaceutically acceptable excipient, binder, and/or diluent. In some embodiments, suitable pharmaceutically acceptable carriers include, but are not limited to, inert solid fillers or diluents and sterile aqueous or organic solutions. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like.

For the purposes of this disclosure, the compounds of the present disclosure can be formulated for administration by a variety of means including orally, parenterally, by inhalation spray, topically, transdermally, buccally, sublingually, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion.

In some embodiments, the pharmaceutical composition is formulated for parenteral administration (such as intravenous injection or infusion, subcutaneous or intramuscular injection). The parenteral formulations can be, for example, an aqueous solution, a suspension, or an emulsion.

In some embodiments, the pharmaceutical composition is formulated for inhalation. The inhalable formulations can be, for example, formulated as a nasal spray, dry powder, or an aerosol administrable through a metered-dose inhaler.

Compounds of the present disclosure can be used alone, in combination with each other, or in combination with one or more additional therapeutic agents, e.g., metformin, recombinant insulin, liraglutide, semaglutide, empagliflozin, paclitaxel, doxorubicin, 5-fluorouracil, tamoxifen, octreotide, etc. When used in combination with one or more additional therapeutic agents, compounds of the present disclosure or pharmaceutical compositions herein can be administered to the subject either concurrently or sequentially in any order with such additional therapeutic agents. In some embodiments, the pharmaceutical composition can comprise one or more compounds of the present disclosure and the one or more additional therapeutic agents in a single composition. In some embodiments, the pharmaceutical composition comprising one or more compounds of the present disclosure can be included in a kit which also comprises a separate pharmaceutical composition comprising the one or more additional therapeutic agents.

As discussed herein, compounds of the present disclosure can sensitize the cancer for chemotherapy treatment. In some embodiments, compounds of the present disclosure can be used in combination with a chemotherapeutic agent, for example, for treating cancer. Any of the known chemotherapeutic agents can be used in combination with one or more compounds of the present disclosure. Non-limiting useful examples of chemotherapeutic agents include antineoplastic agents and combinations thereof, such as DNA alkylating agents (for example cisplatin, oxaliplatin, carboplatin, cyclophosphamide, nitrogen mustards like ifosfamide, bendamustine, melphalan, chlorambucil, busulphan, temozolamide and nitrosoureas like carmustine); antimetabolites (for example gemcitabine and antifolates such as fluoropyrimidines like 5-fluorouracil and tegafur, raltitrexed, methotrexate, cytosine arabinoside, and hydroxyurea); anti-tumour antibiotics (for example anthracyclines like adriamycin, bleomycin, doxorubicin, liposomal doxorubicin, pirarubicin, daunomycin, valrubicin, epirubicin, idarubicin, mitomycin-C, dactinomycin, amrubicin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, irinotecan, topotecan and camptothecin); inhibitors of DNA repair mechanisms such as CHK kinase; DNA-dependent protein kinase inhibitors; inhibitors of poly (ADP-ribose) polymerase (PARP inhibitors, including olaparib); and Hsp90 inhibitors such as tanespimycin and retaspimycin, inhibitors of ATR kinase (such as AZD6738); and inhibitors of WEE1 kinase (such as AZD1775/MK-1775).

In some embodiments, compounds of the present disclosure can also be used for treating type 2 diabetes in combination with one or more additional therapeutic agents useful for treating type 2 diabetes, e.g., metformin, recombinant insulin, liraglutide, semaglutide, empagliflozin etc.

Generally, the compounds of the present disclosure are administered in a therapeutically effective amount. The amount of the compound actually administered will typically be determined by a physician, in the light of the relevant circumstances, including the condition to be treated, the chosen route of administration, the actual compound-administered, the age, weight, and response of the individual patient, the severity of the patient's symptoms, and the like.

### Therapeutic Use

The compounds of the present disclosure find use in any number of methods. For example, in embodiments, the compounds of the present disclosure are useful in methods for modulating aldehyde dehydrogenase e.g., ALDH1a3 and/or ALDH1a2. Accordingly, in some embodiments, the present disclosure provides the use of any one of the foregoing compounds of Formula (I), (II), (III), (IV), (V), or Table 1 or a pharmaceutically acceptable salt thereof, for modulating aldehyde dehydrogenase e.g., ALDH1a3 and/or ALDH1a2 activity. For example, in some embodiments, modulating aldehyde dehydrogenase e.g., ALDH1a3 and/or ALDH1a2 activity is in a mammalian cell. In embodiments, modulating aldehyde dehydrogenase e.g., ALDH1a3 and/or ALDH1a2 activity can be in a subject in need thereof (e.g., a mammalian subject, such as a human) and for treatment of any of the described conditions or diseases.

In some embodiments, the modulating aldehyde dehydrogenase e.g., ALDH1a3 and/or ALDH1a2 activity is binding. In some embodiments, the modulating aldehyde dehydrogenase e.g., ALDH1a3 and/or ALDH1a2 activity is inhibiting the aldehyde dehydrogenase e.g., ALDH1a3 and/or ALDH1a2. In embodiments, the present disclosure provides a method for inhibiting ALDH1a2. In embodiments, ALDH1a2 is selectively inhibited.

In embodiments, the present disclosure provides a method for inhibiting ALDH1a3. In embodiments, ALDH1a3 is selectively inhibited. In embodiments, the present disclosure provides a method for inhibiting both ALDH1a3 and ALDH1a2.

Aldehyde dehydrogenase isoform 1a3 (ALDH1a3) is an isoform/isozyme of the ALDH1a subfamily that is crucial in the biosynthesis of R^{A} and the regulation of R^{A} signaling, and is cell- and disease-specific. ALDH1a3 was known as ALDH6 prior to 2000, and as Raldh3 from 2000-2007 in developmental studies. In normal conditions, ALDH1a3 is only required during embryonic development and is dispensable to healthy adult mice. In adult physiology, humans with homozygous inactivating mutations in Aldh1a3 have been described with incompletely penetrant anopthalmia and no other described pathologies. In contrast to its minor role in normal physiology, ALDH1a3 has recently been shown to be the major determinant of ALDEFLUOR™ reactivity across most cancer types and in de-differentiated pancreatic islet cells. ALDEFLUOR™ activity has long been used as a marker to differentiate aggressive cancer cells from the bulk tumor despite an overlying ignorance regarding if/how ALDEFLUOR™ activity affects tumor progression.

It has been discovered that ALDEFLUOR™ activity driven by ALDH1a3 is a functional driver of cancer aggressiveness, and is critical for tumor progression, metastasis, and resistance to chemotherapy. Thus, human ALDH1a3 (UniProtKB Accession No.: P47895) is a functional driver of chemoresistant and metastatic phenotypes in cancer, including breast cancer. Accordingly, ALDH1a3 represents a potential therapeutic target in multiple pathologies, and targeting ALDH1a3 may overcome the current barrier in treating Stage 3/4 patients whose tumors are resistant to conventional forms of therapy.

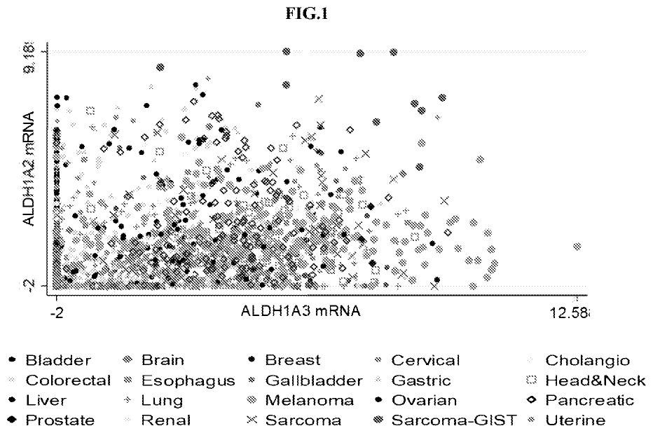

It has been shown, among the tested ALDH isoforms, only ALDH1a2 and ALDH1a3 induce retinoid pathway activation and ALDH1a2 and ALDH1a3 drive retinoid pathway activation in solid tumors in vivo. Further, as shown in FIG. 1 expression of ALDH1a2 and ALDH1a3 mRNA in patient-derived xenograft models from multiple human cancers shows expression of ALDH1a2 and ALDH1a3 enzymes across multiple tumor types.

In some embodiments, the present disclosure provides methods of treating a disease or disorder that is treatable by administration of an aldehyde dehydrogenase inhibitor, the method comprising administering a therapeutically effective amount of one or more compounds of the present disclosure (e.g., compounds of Formula (I), (II) (III), (IV), (V), or Table 1) or a pharmaceutically acceptable salt thereof.

In some embodiments, the disease or disorder is associated with aldehyde dehydrogenase isoform 1a3 (ALDH1a3) in the subject. In some embodiments, the disease or disorder is associated with aldehyde dehydrogenase isoform 1a2 (ALDH1a2) in the subject. In some embodiments, the disease or disorder is associated with aldehyde dehydrogenase isoform 1a3 (ALDH1a3) and 1a2 (ALDH1a2) in the subject. For example, in some embodiments, the disease or disorder is a proliferative disease such as cancer (e.g., as described herein) associated with aldehyde dehydrogenase isoform 1a3 (ALDH1a3) and/or 1a2 (ALDH1a2). In some embodiments, the disease or disorder is a metabolic disease, such as type 2 diabetes associated with aldehyde dehydrogenase isoform 1a3 (ALDH1a3) and/or 1a2 (ALDH1a2). In some embodiments, the disease or disorder is an endothelial cell or smooth muscle cell disease or disorder, such as pulmonary arterial hypertension or neointimal hyperplasia, associated with aldehyde dehydrogenase isoform 1a3 (ALDH1a3) and/or 1a2 (ALDH1a2). In embodiments, the disease or disorder is an immunologically-driven disease or disorder, such as acute graft-vs-host disease or osteoarthritis pain, associated with aldehyde dehydrogenase isoform 1a3 (ALDH1a3) and/or ALDH1a2.

In embodiments, the subject suffers from a disease or disorder associated with aldehyde dehydrogenase, for example, a disease or disorder associated with aldehyde dehydrogenase isoform 1a3 (ALDH1a3) and/or 1a2 (ALDH1a2), in a subject in need thereof. In embodiments, the subject suffers from a proliferative disease such as cancer (e.g., as described herein). In embodiments, the subject suffers from a metabolic disease such as type 2 diabetes. In embodiments, the subject suffers from an endothelial cell or smooth muscle cell disease or disorder, such as pulmonary arterial hypertension or neointimal hyperplasia.

In embodiments, compounds of the present disclosure are used for the treatment and/or prophylaxis of diseases or disorders that are associated with aldehyde dehydrogenase, such as a disease or disorder associated with aldehyde dehydrogenase isoform 1a3 (ALDH1a3) and/or 1a2 (ALDH1a2), including one or more of the following diseases or disorders: proliferative diseases or disorders, metabolic diseases or disorders, endothelial cell or smooth muscle cell diseases or disorders, metastasis, etc. Accordingly, some embodiments of the present disclosure are directed to methods of using one or more compounds of the present disclosure for inhibiting ALDH enzymes such as ALDH1a3 and/or ALDH1a2, and methods of treating or preventing various cancers, cancer metastasis, and/or other ALDH1a2 and/or ALDH1a3-mediated diseases and disorders, such as type 2 diabetes, pulmonary arterial hypertension (PAH) and neointimal hyperplasia (NIH) or as a male contraceptive.

In embodiments, the present disclosure provides a method of using one or more compounds of the present disclosure for antagonizing retinoid pathway. Without wishing to be bound by theories, it is believed that retinoid pathway activation can cause immune tolerance, induction of T_{reg} cells and/or M2 macrophages, and/or effector T cell suppression. Inhibition of ALDH1a2 and/or ALDH1a3 by one or more compounds of the present disclosure can inhibit such retinoid signaling, which can be used to treat diseases or disorders associated with undesired retinoid pathway activation and can restore or activate the subject's immune responses, e.g., against cancer cells. For example, in some embodiments, the compounds of the present disclosure can be used in combination with an immunotherapy (e.g., an immune checkpoint inhibitor) to treat diseases or disorders that are unresponsive to the immunotherapy or to treat a subject who has developed resistance to the immunotherapy.

Compounds of the present disclosure can inhibit ALDH1a2 and/or ALDH1a3, can inhibit retinoid signaling, and can be used to treat various diseases or disorders associated with ALDH1a2 and/or ALDH1a3, and diseases or disorders associated with retinoid pathway activation.

In some embodiments, the present disclosure provides a method of treating cancer in a subject in need thereof. In some embodiments, the method comprises administering to the subject a therapeutically effective amount of a compound of the present disclosure (e.g., compounds of Formula (I), (II) (III), (IV), (V), or Table 1) or a pharmaceutically acceptable salt thereof or a therapeutically effective amount of a pharmaceutical composition described herein.

Many cancer types were shown to have ALDH1a3 activities which can be inhibited by representative compounds of the present disclosure. Also, as shown in FIG. 1, many cancer types were shown to express ALDH1a2 and ALDH1a3 enzymes, the activities of which can be inhibited by representative compounds of the present disclosure. In some embodiments, the cancer is a solid cancer. In some embodiments, the cancer is metastatic cancer or chemoresistant cancer. In some embodiments, the cancer is resistant to one or more immunotherapy, such as an immune checkpoint inhibitor, such as an anti-PD-1 antibody, anti-PD-L1 antibody, anti-CTLA4 antibody, IL-2, autologous T cell therapy, bispecific antibody therapy, anti-TGFβ antibody, a JAK/STAT inhibitor, or any combination thereof.

In some embodiments, the cancer is a breast cancer, colorectal cancer, kidney cancer, ovarian cancer, gastric cancer, thyroid cancer, urothelial cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, hematologic cancer, sarcoma, gastrointestinal stromal tumor, peripheral nerve sheath tumor, myeloma, and/or endometrial cancer.

In some embodiments, the cancer is a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, and/or sarcoma. In some embodiments, the cancer is breast cancer (e.g., (e.g., ER negative breast cancer, triple negative breast cancer, basal-like breast cancers, or HER2-positive breast cancers), clear cell renal cell cancer, gastric cancer, bladder cancer, ovarian cancer, squamous cell lung cancer, colorectal cancer or glioma (e.g., low-grade glioma) cancer. In some embodiments, the cancer can also be gastrointestinal stromal tumor, peripheral nerve sheath tumor, myeloma, leukemia, lymphoma and/or endometrial cancer. In some embodiments, the cancer can also be any of those shown in FIG. 7 herein, such as a bladder, brain, breast, cervical, cholangio, esophagus, gallbladder, gastric, head and neck, liver, lung, melanoma, ovarian, pancreatic, prostate, renal, sarcoma, sarcoma-GIST, and/or uterine cancer.

In some embodiments, the cancer has established metastasis. In some embodiments, the cancer has not metastasized prior to treatment with the methods herein, and the method comprises administering an effective amount of one or more compounds of the present disclosure to delay or prevent metastasis of the cancer. In embodiments described herein, the cancer is associated with ALDH1a3 and/or ALDH1a2 activities, such as having higher expression level compared to a control, and/or having cancer cells with ALDH1a3 and/or ALDH1a2 activities, e.g., positive in Aldefluor™ assay, which can be reduced with an ALDH1a3 and/or ALDH1a2 inhibitor or genetic knockout or knockdown, as applicable. In some embodiments, the method further comprises administering to the subject an effective amount of a second anti-cancer therapy, such as a chemotherapeutic agent (e.g., described herein, such as paclitaxel), a receptor tyrosine kinase inhibitor, or a therapeutic antibody. In some embodiments, the method further comprises administering to the subject an effective amount of an immunotherapy, such as an immune check point inhibitor. Suitable immunotherapy for the methods described herein is not particularly limited and can include any of those known in the art, which can include for example, anti-PD-1 antibody (e.g., nivolumab, pembrolizumab, lambrolizumab, pidilizumab, BMS-936559, or AMP-224), anti-PD-L1 antibody (e.g., atezolizumab, durvalumab, avelumab, YW243.55.S70, MEDI-4736, MSB-0010718C, LY3300054, BMS-936559, MPDL3280A, or MDX-1105), IL-2, autologous T cell therapy, bispecific antibody therapy, anti-TGFβ antibody, a JAK/STAT inhibitor, or any combination thereof.

In some embodiments, the present disclosure provides a method of treating metastatic cancer in a subject in need thereof, the method comprising administering to the subject a therapeutically effective amount of a compound of the present disclosure (e.g., compounds of Formula (I), (II), (III), (IV), (V), or Table 1) or a pharmaceutically acceptable salt thereof) or a therapeutically effective amount of a pharmaceutical composition described herein. In some embodiments, the metastatic cancer is a solid cancer. In some embodiments, the metastatic cancer can be a metastatic breast cancer, metastatic colorectal cancer, metastatic kidney cancer, metastatic ovarian cancer, metastatic gastric cancer, metastatic thyroid cancer, metastatic testicular cancer, metastatic cervical cancer, metastatic nasopharyngeal cancer, metastatic esophageal cancer, metastatic bile duct cancer, metastatic lung cancer, metastatic pancreatic cancer, metastatic prostate cancer, metastatic bone cancer, metastatic blood cancer, metastatic brain cancer, metastatic liver cancer, metastatic mesothelioma, metastatic melanoma, and/or metastatic sarcoma. In some embodiments, the cancer is metastatic breast (e.g., ER negative breast cancer, triple negative breast cancer, basal-like breast cancers, or HER2-positive breast cancers), clear cell renal cell, gastric, bladder, ovarian, squamous cell lung, colorectal or glioma (e.g., low-grade glioma) cancer. In some embodiments, the metastatic cancer can also be a metastatic cancer selected from gastrointestinal stromal tumor, peripheral nerve sheath tumor, myeloma, and/or endometrial cancer. In some embodiments, the metastatic cancer can also be a metastatic cancer selected from a bladder, brain, breast, cervical, cholangio, esophagus, gallbladder, gastric, head and neck, liver, lung, melanoma, ovarian, pancreatic, prostate, renal, sarcoma, sarcoma-GIST, and/or uterine cancer. In some embodiments, the metastatic cancer is associated with ALDH1a3 and/or ALDH1a2 activities. In some embodiments, the metastatic cancer can be breast cancer with established lung metastasis, colorectal metastasis, and/or bone metastasis. In some embodiments, the method further comprises administering to the subject an effective amount of a second anti-cancer therapy, such as a chemotherapeutic agent (e.g., described herein, such as paclitaxel), a receptor tyrosine kinase inhibitor, or a therapeutic antibody.

In some embodiments, the present disclosure provides a method of treating chemoresistant cancer in a subject in need thereof, the method comprising administering to the subject a therapeutically effective amount of a compound of the present disclosure (e.g., compounds of Formula (I), (II), (III), (IV), (V), or Table 1) or a pharmaceutically acceptable salt thereof) or a therapeutically effective amount of a pharmaceutical composition described herein. “Chemoresistant cancer,” as used herein, refers to a cancer that does not respond to treatment with one or more chemotherapeutic agents. “Chemoresistant cancers” include those that are non-responsive to treatment with one or more therapeutic agents at the beginning of treatment, and those that become non-responsive to treatment with one or more therapeutic agents during treatment. Chemoresistant cancers that are particularly suitable for treatment using the methods described herein include, but are not limited to, cancers that are resistant to treatment with paclitaxel and/or doxorubicin. In some embodiments, the chemoresistant cancer is a solid cancer. In some embodiments, the chemoresistant cancer can be a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, and/or sarcoma. In some embodiments, the cancer can also be gastrointestinal stromal tumor, peripheral nerve sheath tumor, myeloma, and/or endometrial cancer. In some embodiments, the cancer can also be a bladder, brain, breast, cervical, cholangio, esophagus, gallbladder, gastric, head and neck, liver, lung, melanoma, ovarian, pancreatic, prostate, renal, sarcoma, sarcoma-GIST, and/or uterine cancer. In some embodiments, the cancer can be a breast (e.g., triple negative breast), clear cell renal cell, gastric, bladder, ovarian, squamous cell lung, colorectal or glioma (e.g., low-grade glioma) cancer. In some embodiments, the chemoresistant cancer is associated with ALDH1a3 and/or ALDH1a2 activities. In some embodiments, the method further comprises administering to the subject an effective amount of a second anti-cancer therapy, such as a chemotherapeutic agent (e.g., described herein, such as paclitaxel), a receptor tyrosine kinase inhibitor, or a therapeutic antibody.

In some embodiments, the present disclosure provides a method of sensitizing cancer for chemotherapy in a subject in need thereof, the method comprising administering to the subject an effective amount of a compound of the present disclosure (e.g., compounds of Formula (I), (II) (III), (IV), (V), or Table 1) or a pharmaceutically acceptable salt thereof) or an effective amount of a pharmaceutical composition described herein. Typically, the method can cause the cancer more responsive to treatment with chemotherapeutic agent. In some embodiments, the cancer is a solid cancer. In some embodiments, the cancer can be a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, and/or sarcoma. In some embodiments, the cancer can also be gastrointestinal stromal tumor, peripheral nerve sheath tumor, myeloma, and/or endometrial cancer. In some embodiments, the cancer can also be a bladder, brain, breast, cervical, cholangio, esophagus, gallbladder, gastric, head and neck, liver, lung, melanoma, ovarian, pancreatic, prostate, renal, sarcoma, sarcoma-GIST, and/or uterine cancer. In some embodiments, the cancer is associated with ALDH1a3 and/or ALDH1a2 activities. In some embodiments, the method further comprises administering to the subject an effective amount of a second anti-cancer therapy, such as a chemotherapeutic agent (e.g., described herein, such as paclitaxel), a receptor tyrosine kinase inhibitor, or a therapeutic antibody.

In some embodiments, the present disclosure provides a method of treating or preventing metastasis of a cancer in a subject in need thereof, the method comprising administering to the subject an effective amount of a compound of the present disclosure (e.g., compounds of Formula (I), (II) (III), (IV), (V), or Table 1) or a pharmaceutically acceptable salt thereof) or an effective amount of a pharmaceutical composition described herein. In some embodiments, the cancer is a solid cancer. In some embodiments, the cancer can be a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, and/or sarcoma. In some embodiments, the cancer can also be gastrointestinal stromal tumor, peripheral nerve sheath tumor, myeloma, and/or endometrial cancer. In some embodiments, the cancer can also be a bladder, brain, breast, cervical, cholangio, esophagus, gallbladder, gastric, head and neck, liver, lung, melanoma, ovarian, pancreatic, prostate, renal, sarcoma, sarcoma-GIST, and/or uterine cancer. In some embodiments, the cancer is associated with ALDH1a3 and/or ALDH1a2 activities. In some embodiments, the cancer has established metastasis. In some embodiments, the cancer has not metastasized prior to treatment with the methods herein, and the method delays or prevents metastasis of the cancer. In some embodiments, the method further comprises administering to the subject an effective amount of a second anti-cancer therapy, such as a chemotherapeutic agent (e.g., described herein, such as paclitaxel), a receptor tyrosine kinase inhibitor, or a therapeutic antibody.

In some embodiments, the cancer is unresponsive to one or more immunotherapy, e.g., an anti-PD-1, anti-CTLA4, anti-LAG-3, anti-TIGIT or anti-PD-L1 antibody. In some embodiments, the subject has developed resistance to one or more immunotherapy, e.g., an anti-PD-1 or anti-PD-L1 antibody. In some embodiments, the method further comprises administering to the subject one or more immunotherapy (e.g., as described herein).

In some embodiments, the present disclosure provides a method of antagonizing the retinoid pathway in a subject in need thereof. In some embodiments, the method comprises administering to the subject an effective amount of an ALDH1a2 inhibitor. In some embodiments, the method comprises administering to the subject an effective amount of an ALDH1a3 inhibitor. In embodiments, the method of antagonizing the retinoid pathway comprises administering to the subject an effective amount of an ALDH1a2 inhibitor and an ALDH1a3 inhibitor, for example, an effective amount of a dual inhibitor of ALDH1a2 and ALDH1a3 or a combination of one or more ALDH1a2 inhibitor and one or more ALDH1a3 inhibitor. In some embodiments, the method comprising administering to the subject an effective amount of a compound of the present disclosure (e.g., compounds of Formula (I), (II), (III), (IV), (V), or Table 1) or a pharmaceutically acceptable salt thereof) or an effective amount of a pharmaceutical composition described herein. In some embodiments, the subject suffers from a disease or disorder associated with retinoid pathway activation (e.g., any of those described herein).

In some embodiments, the present disclosure provides a method of inhibiting T_{reg} cell and/or M2 macrophage formation in a subject in need thereof. In some embodiments, the method comprises administering to the subject an effective amount of an ALDH1a2 inhibitor. In some embodiments, the method comprises administering to the subject an effective amount of an ALDH1a3 inhibitor. In some embodiments, the method of inhibiting Ts cell and/or M2 macrophage formation comprises administering to the subject an effective amount of an ALDH1a2 inhibitor and an ALDH1a3 inhibitor, for example, an effective amount of a dual inhibitor of ALDH1a2 and ALDH1a3 or a combination of one or more ALDH1a2 inhibitor and one or more ALDH1a3 inhibitor. In some embodiments, the method comprising administering to the subject an effective amount of a compound of the present disclosure (e.g., compounds of Formula (I), (H), (III), (IV), (V), or Table 1) or a pharmaceutically acceptable salt thereof), or an effective amount of a pharmaceutical composition described herein. In some embodiments, the subject is characterized as having a cancer (e.g., described herein) unresponsive to one or more immunotherapy or the subject has developed resistance to one or more immunotherapy. In some embodiments, the cancer is a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, sarcoma, gastrointestinal stromal tumor, peripheral nerve sheath tumor, myeloma, and/or endometrial cancer. In some embodiments, the cancer can also be a bladder, brain, breast, cervical, cholangio, esophagus, gallbladder, gastric, head and neck, liver, lung, melanoma, ovarian, pancreatic, prostate, renal, sarcoma, sarcoma-GIST, and/or uterine cancer. In some embodiments, the cancer is unresponsive to one or more immunotherapy, such as an anti-PD-1 antibody, anti-PDL-1 antibody, anti-CTLA4 antibody, IL-2, autologous T cell therapy, bispecific antibody therapy, anti-TGFβ antibody, a JAK/STAT inhibitor, or any combination thereof. In some embodiments, the subject has developed resistance to one or more immunotherapy, such as an anti-PD-1 antibody, anti-PD-L1 antibody, anti-CTLA4 antibody, IL-2, autologous T cell therapy, bispecific antibody therapy, anti-TGFβ antibody, a JAK/STAT inhibitor, or any combination thereof. In some embodiments, the method further comprises administering to the subject one or more immunotherapy, such as an immune checkpoint inhibitor. In some embodiments, the method further comprises administering to the subject an anti-PD-1 antibody, anti-PD-L1 antibody, anti-CTLA4 antibody, IL-2, autologous T cell therapy, bispecific antibody therapy, anti-TGFβ antibody, a JAK/STAT inhibitor, or any combination thereof.

In some embodiments, the present disclosure provides a method of treating a disease or disorder associated with the retinoid pathway activation in a subject in need thereof. In some embodiments, the method comprises administering to the subject an effective amount of an ALDH1a2 inhibitor. In some embodiments, the method comprises administering to the subject an effective amount of an ALDH1a3 inhibitor. In some embodiments, the method of treating a disease or disorder associated with the retinoid pathway activation comprises administering to the subject an effective amount of an ALDH1a2 inhibitor and an ALDH1a3 inhibitor, for example, an effective amount of a dual inhibitor of ALDH1a2 and ALDH1a3 or a combination of one or more ALDH1a2 inhibitor and one or more ALDH1a3 inhibitor. In some embodiments, the method comprising administering to the subject an effective amount of a compound of the present disclosure (e.g., compounds of Formula (I), (II), (III), (IV), (V), or Table 1) or a pharmaceutically acceptable salt thereof) or an effective amount of a pharmaceutical composition described herein. In some embodiments, the disease or disorder is associate with immunosuppression or immunotolerance of the subject. In some embodiments, the disease or disorder is associate with induction of T_{reg} cells and/or M2 macrophages, and/or effector T cell suppression in the subject. In some embodiments, the disease or disorder is cancer (e.g., described herein). In some embodiments, the cancer is a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, sarcoma, gastrointestinal stromal tumor, peripheral nerve sheath tumor, myeloma, and/or endometrial cancer. In some embodiments, the cancer can also be a bladder, brain, breast, cervical, cholangio, esophagus, gallbladder, gastric, head and neck, liver, lung, melanoma, ovarian, pancreatic, prostate, renal, sarcoma, sarcoma-GIST, and/or uterine cancer. In some embodiments, the cancer is unresponsive to one or more immunotherapy, such as an anti-PD-1 antibody, anti-PDL-1 antibody, anti-CTLA4 antibody, IL-2, autologous T cell therapy, bispecific antibody therapy, anti-TGFβ antibody, a JAK/STAT inhibitor, or any combination thereof. In some embodiments, the subject has developed resistance to one or more immunotherapy, such as an anti-PD-1 antibody, anti-PDL-1 antibody, anti-CTLA4 antibody, IL-2, autologous T cell therapy, bispecific antibody therapy, anti-TGFβ antibody, a JAK/STAT inhibitor, or any combination thereof. In some embodiments, the method further comprises administering to the subject one or more immunotherapy, such as an immune checkpoint inhibitor. In some embodiments, the method further comprises administering to the subject an anti-PD-1 antibody, anti-PD-L1 antibody, anti-CTLA4 antibody, IL-2, autologous T cell therapy, bispecific antibody therapy, anti-TGFβ antibody, a JAK/STAT inhibitor, or any combination thereof.

In some embodiments, the present disclosure provides a method of treating cancer in a subject in need thereof, wherein the cancer is unresponsive to one or more immunotherapy or the subject has developed resistance to one or more immunotherapy, the method comprising administering to the subject an effective amount of a compound of the present disclosure (e.g., compounds of Formula (I), (II), (III), (IV), (V), or Table 1) or a pharmaceutically acceptable salt thereof) or an effective amount of a pharmaceutical composition described herein. In some embodiments, the cancer is unresponsive to one or more immunotherapy, such as an anti-PD-1 antibody, anti-PD-L1 antibody, anti-CTLA4 antibody, IL-2, autologous T cell therapy, bispecific antibody therapy, anti-TGFβ antibody, a JAK/STAT inhibitor, or any combination thereof. In some embodiments, the cancer is unresponsive to treatment with anti-PD-1 or anti-PD-L1 antibodies. In some embodiments, the subject has developed resistance to one or more immunotherapy, such as an anti-PD-1 antibody, anti-PDL-1 antibody, anti-CTLA4 antibody, IL-2, autologous T cell therapy, bispecific antibody therapy, anti-TGFβ antibody, a JAK/STAT inhibitor, or any combination thereof. In some embodiments, the subject has developed resistance to anti-PD-1 or anti-PD-L1 antibodies based treatment. In some embodiments, the method further comprises administering to the subject one or more immunotherapy, such as an immune checkpoint inhibitor. In some embodiments, the method further comprises administering to the subject an anti-PD-1 antibody, anti-PDL-1 antibody, anti-CTLA4 antibody, IL-2, autologous T cell therapy, bispecific antibody therapy, anti-TGFβ antibody, a JAK/STAT inhibitor, or any combination thereof. In some embodiments, the cancer is a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, sarcoma, gastrointestinal stromal tumor, peripheral nerve sheath tumor, myeloma, and/or endometrial cancer. In some embodiments, the cancer can also be a bladder, brain, breast, cervical, cholangio, esophagus, gallbladder, gastric, head and neck, liver, lung, melanoma, ovarian, pancreatic, prostate, renal, sarcoma, sarcoma-GIST, and/or uterine cancer.

In some embodiments, the present disclosure provides a method of treating cancer in a subject in need thereof, the method comprising administering to the subject an effective amount of a compound of the present disclosure (e.g., compounds of Formula (I), (II), (III), (IV), (V), or Table 1) or a pharmaceutically acceptable salt thereof) or an effective amount of a pharmaceutical composition described herein, in combination with an immunotherapy, such as an immune checkpoint inhibitor. In some embodiments, the immunotherapy comprises administering to the subject an anti-PD-1 antibody, anti-PD-L1 antibody, anti-CTLA4 antibody, IL-2, autologous T cell therapy, bispecific antibody therapy, anti-TGFβ antibody, a JAK/STAT inhibitor, or any combination thereof. In some embodiments, the cancer is a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, sarcoma, gastrointestinal stromal tumor, peripheral nerve sheath tumor, myeloma, and/or endometrial cancer. In some embodiments, the cancer can also be a bladder, brain, breast, cervical, cholangio, esophagus, gallbladder, gastric, head and neck, liver, lung, melanoma, ovarian, pancreatic, prostate, renal, sarcoma, sarcoma-GIST, and/or uterine cancer.

In some embodiments, the present disclosure provides a method of treating or preventing a metabolic disease, such as Type 2 Diabetes in a subject in need thereof, the method comprising administering to the subject a therapeutically effective amount of a compound of the present disclosure (e.g., compounds of Formula (I), (II), (III), (IV), (V), or Table 1) or a pharmaceutically acceptable salt thereof) or a therapeutically effective amount of a pharmaceutical composition described herein.

As discussed herein, metabolic diseases such as type 2 diabetes are associated with a pathology driven by ALDH1a3 activities. In some embodiments, the method further comprises administering to the subject an effective amount of an additional anti-metabolic diseases agents, such as anti-type 2 diabetes agent. Suitable additional anti-metabolic diseases agents include without limitation an incretin mimic, recombinant insulin, a biguanide, SGLT2 inhibitors, a therapeutic antibody, etc. Any of the known Type 2 Diabetes treatments can be used in combination with the compounds of the present disclosure, for example, for treating Type 2 Diabetes (e.g., described herein) or treating or preventing other metabolic syndromes.

In some embodiments, the present disclosure provides a method of treating an endothelial cell or smooth muscle cell disease or disorder, such as pulmonary arterial hypertension or neointimal hyperplasia, in a subject in need thereof, the method comprising administering to the subject a therapeutically effective amount of a compound of the present disclosure (e.g., compounds of Formula (I), (II), (III), (IV), (V), or Table 1) or a pharmaceutically acceptable salt thereof) or a therapeutically effective amount of a pharmaceutical composition described herein. In some embodiments, the endothelial cell or smooth muscle cell disease or disorder is associated with a pathology driven by ALDH1a3 activities. In some embodiments, the endothelial cell or smooth muscle cell disease or disorder is pulmonary arterial hypertension. In some embodiments, the endothelial cell or smooth muscle cell disease or disorder is neointimal hyperplasia.

Also provided herein is a method of inhibiting the proliferation of a cancer cell (e.g., a metastatic cancer cell, a chemoresistant cancer cell). The method comprises administering to the cell (e.g., an effective amount of) one or more compounds of the present disclosure. In a particular embodiment, the cancer cell is a breast cancer cell (e.g., a basal-like breast cancer cell or a HER-2 positive breast cancer cell). The cell can be a cultured cell (e.g., cell line) or a cell in a subject. In a particular embodiment, the cell is present in a human subject (e.g., a human subject with a cancer).

In some embodiments, the present disclosure provides a method of male contraception, the method comprising administering to a subject in need thereof an effective amount of a compound of the present disclosure (e.g., compounds of Formula (I), (II), (III), (IV), (V), or Table 1) or a pharmaceutically acceptable salt thereof) or an effective amount of a pharmaceutical composition described herein.

In embodiments, the compound of the present disclosure recited in the methods herein is a compound of the present disclosure having an IC50 value of less than 250 nM (e.g., in particular embodiments, less than 100 nM, such as about 1-100 nM, about 10-100 nM, about 10-50 nM, about 20-100 nM, about 20-50 nM, etc.) in inhibiting hALDH1a3 when measured by the method described herein according to Example 12.

In some embodiments, the compound of the present disclosure recited in the methods herein is a compound of the present disclosure having an IC50 value of less than 250 nM (e.g., in particular embodiments, less than 100 nM, such as about 1-100 nM, about 10-100 nM, about 10-50 nM, about 20-100 nM, about 20-50 nM, etc.) in inhibiting hALDH1a2 when measured by the method described herein according to Example 13.

In some embodiments, the compound of the present disclosure recited in the methods herein, e.g., methods for inhibiting retinoid pathway or for treating a diseases or disorder associated with the retinoid pathway, such as various cancer discussed herein, diseases or disorders associated with immune tolerance, or for male contraception, can be any compound of the present disclosure having an IC50 value of less than 250 nM (e.g., in particular embodiments, less than 100 nM, such as about 1-100 nM, about 10-100 nM, about 10-50 nM, about 20-100 nM, about 20-50 nM, etc.) in inhibiting hALDH1a3 and/or hALDH1a2 when measured by the method described herein according to Example's 12 and 13.

In embodiments, the compound of the present disclosure recited in the methods herein is a compound of the present disclosure that selectively inhibits ALDH1a2. For example in embodiments, ALDH1a2 is selectively inhibited over ALDH1a3 e.g., by at least about 5-fold, at least about 10-fold, at least about 15-fold, or at least about 20-fold. In embodiments, the compound of the present disclosure has an IC50 value of A, B, or C activity (e.g., as defined herein in Example 13) in inhibiting ALDH1a2 and an IC50 value of D or E activity (e.g., as defined herein in Example 12) in inhibiting ALDH1a3. In embodiments, the ALDH1a2 is hALDH1a2. In embodiments, the ALDH1a3 is hALDH1a3.

In embodiments, the compound of the present disclosure recited in the methods herein is a compound of the present disclosure that selectively inhibits ALDH1a3. For example in embodiments, ALDH1a3 is selectively inhibited over ALDH1a2 e.g., by at least about 5-fold, at least about 10-fold, at least about 15-fold, or at least about 20-fold. In embodiments, the compound of the present disclosure has an IC50 value of A, B, or C activity (e.g., as defined herein in Example 12) in inhibiting ALDH1a3 and an IC50 value of D or E activity (e.g., as defined herein in Example 13) in inhibiting ALDH1a2. In embodiments, the ALDH1a2 is hALDH1a2. In embodiments, the ALDH1a3 is hALDH1a3.

As used herein, the term “compound(s) of the present disclosure” refers to any of the compounds described herein according to Formula (I) (e.g., Formula (I), (II), (III), (IV), or (V)) or any of the compounds in Table 1, isotopically labeled compound(s) thereof (such as a deuterated analog wherein one or more of the hydrogen atoms is/are substituted with a deuterium atom with an abundance above its natural abundance), possible stereoisomers thereof (including diastereoisomers, enantiomers, and racemic mixtures), tautomers thereof, conformational isomers thereof, and/or possible pharmaceutically acceptable salts thereof (e.g., acid addition salt such as HCl salt or base addition salt such as Na salt). Hydrates and solvates of the compounds of the present disclosure are considered compositions of the present disclosure, wherein the compound(s) is in association with water or solvent, respectively.

### EMBODIMENTS

Embodiment 1.1. A compound of Formula (T)

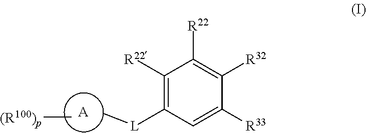

or a pharmaceutically acceptable salt thereof,
wherein:
   ring A is a heterocyclyl, heteroaryl, or aryl;
   L is —NH—, —C(O)—NH—, —C(O)—N(R^{A})-alkylene-O—, -(alkylene)-N(R^{A})-alkylene-O—, —S(O)NH—, —S(O)₂NH—, —S(O)—, or —S(O)₂—,
   R²² is halo, —CO—N(R^{A})₂, —CN, —C₁₋₆ alkyl, —C₁₋₆ alkyl-CN, —C₁₋₆ haloalkyl, or carbocyclyl;
   R^{22′} is H, —CN, halo, —C₁₋₆ alkyl, or —C₁₋₆ haloalkyl; or
   R²² and R^{22′} are joined to form a heteroaryl, carbocyclyl, or heterocyclyl, each of which may be substituted with one or more halo;
   R³² and R³³ are joined to form a heterocyclyl and substituted with oxo; and
wherein the heterocyclyl may be further optionally substituted with one or more R¹⁰¹;
   p is 0, 1 or 2;
   each R¹⁰⁰ is independently halo, —C₁₋₆alkyl, —C₁₋₆alkylene-carbocyclyl, —C₁₋₆ alkylene-heterocyclyl, —C₁₋₆ haloalkyl, or aryl, wherein aryl is optionally substituted with one or more R¹⁰¹;
   each R¹⁰¹ is independently halo, —CO—N(R^{A})₂, —CN, hydroxy, —C₁₋₆ alkyl or —C₁₋₆ haloalkyl; and
   R^{A} is H, —C₁₋₆ alkyl, or aryl.

Embodiment 1.2. The compound of embodiment 1.1, wherein the compound of Formula (I) is a compound of Formula (H).

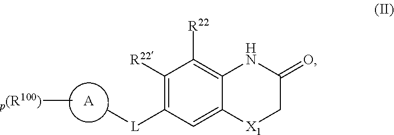

wherein:
   X₁ is —S—, —S(O)₂—, or —CH₂—;
ring A is heteroaryl or aryl;
L is —C(O)—NH—, —C(O)—N(R^{A})-alkylene-O—, -(alkylene)-N(R^{A})-alkylene-O—;
R²² is halo, —CN, —C₁₋₆ alkyl, —C₁₋₆ alkyl-CN, —C₁₋₆haloalkyl;
R^{22′} is H;
p is 0, 1 or 2;
each R¹⁰⁰ is independently halo, —C₁₋₆ alkyl, —C₁₋₆ alkylene-carbocyclyl, —C₁₋₆ alkylene-heterocyclyl, —C₁₋₆ haloalkyl, or aryl, wherein aryl is optionally substituted with one or more R¹⁰¹;
each R¹⁰¹ is independently halo, —CN, hydroxy, —C₁₋₆ alkyl; and
R^{A} is H or —C₁₋₆ alkyl.

Embodiment 1.3. The compound of embodiment 1.1 or 1.2, wherein L is —C(O)—NH—, —C(O)—N(R^{A})-alkylene-O—, or -(alkylene)-N(R^{A})-alkylene-O—.

Embodiment 1.4. The compound of any one of embodiments 1.1-1.3, wherein ring A is a 5 or 6-membered heteroaryl, phenyl, a 5,6-bicyclic heteroaryl, a 5,6-bicyclic heterocyclyl, a 6,6-bicyclic heterocyclyl, a 6,6-bicyclic heteroaryl, or a 3-8 membered heterocyclyl.

Embodiment 1.5. The compound of embodiment 1.4, wherein ring A is a 5 or 6-membered heteroaryl or phenyl.

Embodiment 1.6. The compound of embodiment 1.5, wherein ring A is pyridyl, phenyl, or thiazolyl.

Embodiment 1.7. The compound of any one of embodiments 1.1-1.6, wherein ring A is

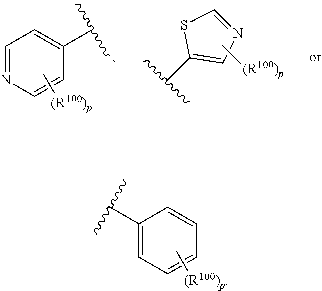

Embodiment 1.8. The compound of any one of embodiments 1.1-1.7, wherein each R¹⁰⁰ is independently halo, —C₁₋₆ haloalkyl, or aryl, wherein aryl is optionally substituted with one or more R¹⁰¹.

Embodiment 1.9. The compound of any one of embodiments 1.1-1.8, wherein ring A-(R¹⁰⁰)ₚ is:

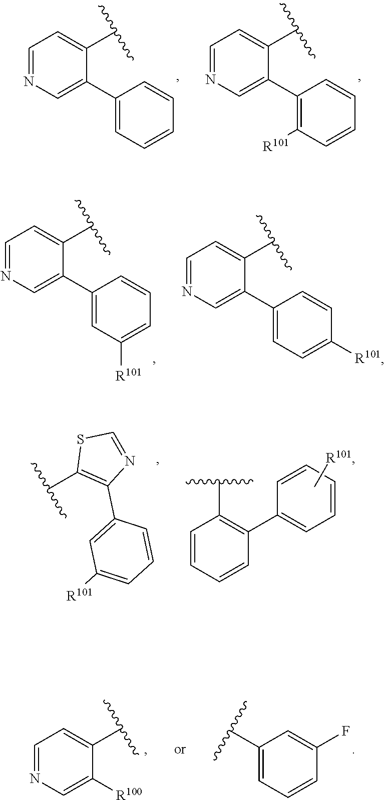

Embodiment 1.10. The compound of embodiment 1.8 or 1.9, wherein R¹⁰¹ is fluoro, hydroxy, —CN or methyl.

Embodiment 1.11. The compound of any one of embodiments 1.1-1.10, wherein R²² is halo, —CN, —C₁₋₆alkyl, or C₁₋₆ haloalkyl.

Embodiment 1.12. The compound of embodiment 1.11, wherein R²² is halo or —C₁₋₆ alkyl.

Embodiment 1.13. The compound of embodiment 1.12, wherein R²² is methyl or chloro.

Embodiment 1.14. The compound of any one of embodiments 1.1-1.13, wherein R^{22′} is —H, halo, —C₁₋₆alkyl, or —C₁₋₆haloalkyl.

Embodiment 1.15. The compound of any one of embodiments 1.1-1.14, wherein R^{22′} is —H or F.

Embodiment 1.16. The compound of embodiment 1.15, wherein R^{22′} is —H.

Embodiment 1.17. A compound selected from:

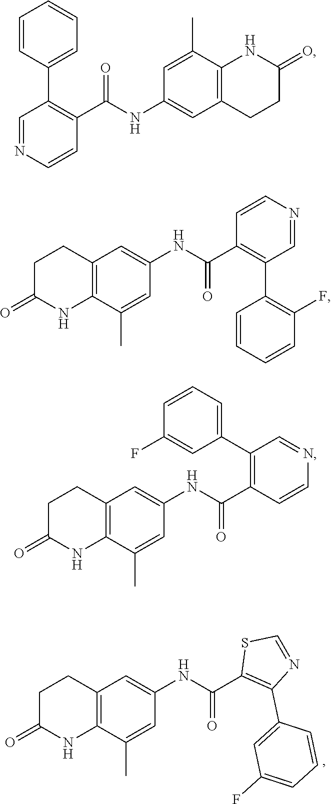 [SMILES: Cc1cc(NC(=O)c2ccncc2-c2ccccc2)cc2c1NC(=O)CC2 ; Cc1cc(NC(=O)c2ccncc2-c2ccccc2F)cc2c1NC(=O)CC2 ; Cc1cc(NC(=O)c2ccncc2-c2cccc(F)c2)cc2c1NC(=O)CC2 ; Cc1cc(NC(=O)c2scnc2-c2cccc(F)c2)cc2c1NC(=O)CC2]

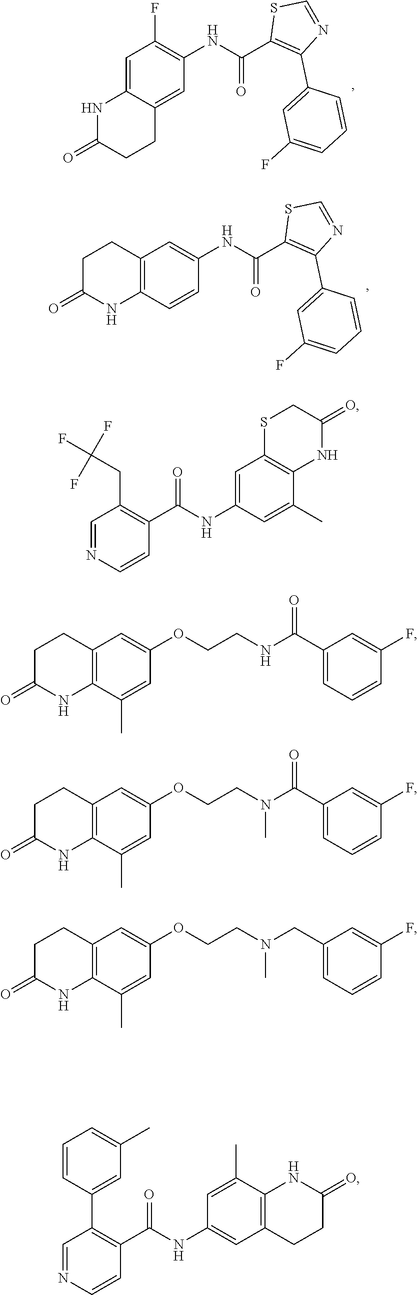

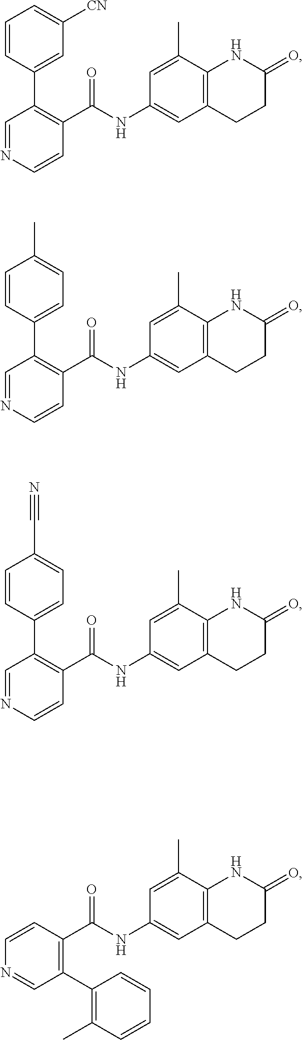 [SMILES: Cc1cc(NC(=O)c2ccncc2-c2cccc(C#N)c2)cc2c1NC(=O)CC2 ; Cc1ccc(-c2cnccc2C(=O)Nc2cc(C)c3c(c2)CCC(=O)N3)cc1 ; Cc1cc(NC(=O)c2ccncc2-c2ccc(C#N)cc2)cc2c1NC(=O)CC2 ; Cc1ccccc1-c1cnccc1C(=O)Nc1cc(C)c2c(c1)CCC(=O)N2]

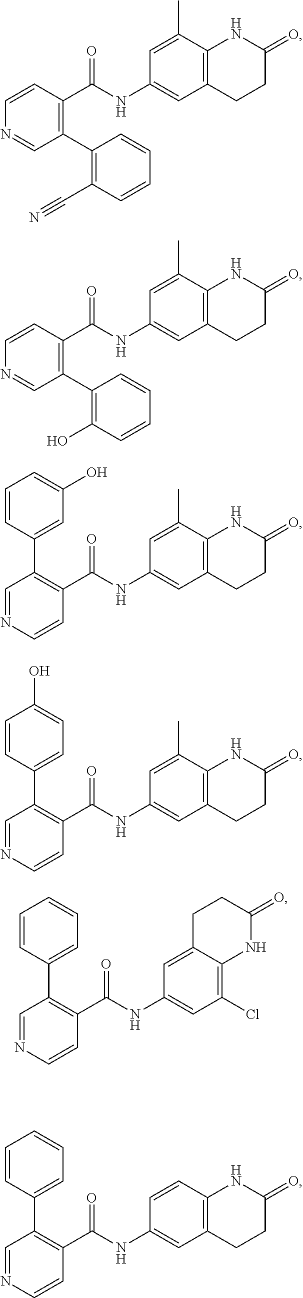

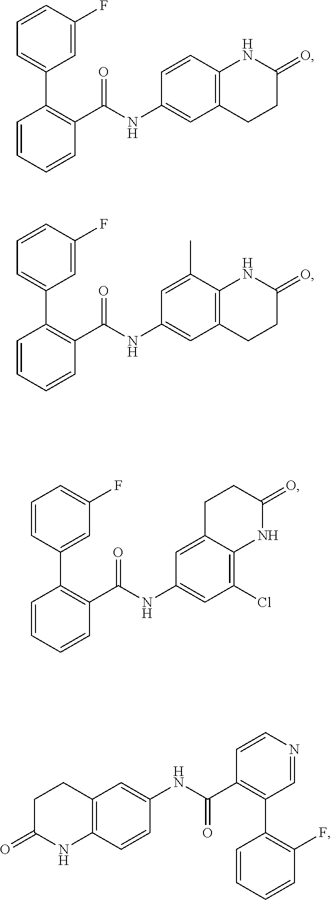 [SMILES: O=C1CCc2cc(NC(=O)c3ccccc3-c3cccc(F)c3)ccc2N1 ; Cc1cc(NC(=O)c2ccccc2-c2cccc(F)c2)cc2c1NC(=O)CC2 ; O=C1CCc2cc(NC(=O)c3ccccc3-c3cccc(F)c3)cc(Cl)c2N1 ; O=C1CCc2cc(NC(=O)c3ccncc3-c3ccccc3F)ccc2N1]

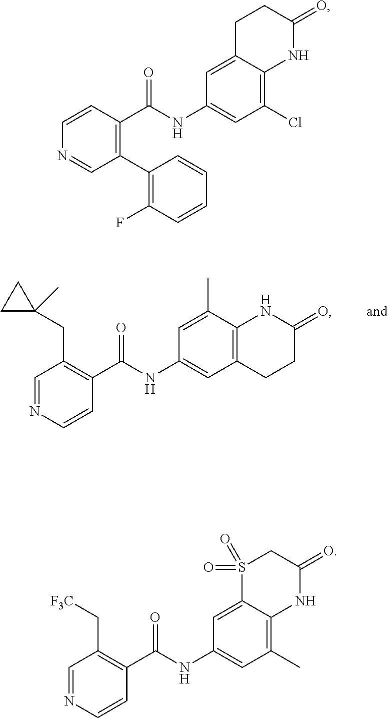 [SMILES: O=C1CCc2cc(NC(=O)c3ccncc3-c3ccccc3F)cc(Cl)c2N1 ; Cc1cc(NC(=O)c2ccncc2CC2(C)CC2)cc2c1NC(=O)CC2 ; "and" ; Cc1cc(NC(=O)c2ccncc2CC(F)(F)F)cc2c1NC(=O)CS2(=O)=O]

Embodiment 1.18. A pharmaceutical composition comprising the compound of any one of embodiments 1.1-1.17, or a pharmaceutically acceptable salt thereof, and a pharmaceutically acceptable carrier.

Embodiment 1.19. A method of treating cancer in a subject in need thereof, the method comprising administering to the subject a therapeutically effective amount of the compound of any one of embodiments 1.1-1.17 or a pharmaceutically acceptable salt thereof, or the pharmaceutical composition of embodiment 1.18.

Embodiment 1.20. A method of treating metastatic cancer or chemoresistant cancer in a subject in need thereof, the method comprising administering to the subject a therapeutically effective amount of the compound of any one of embodiments 1.1-1.17 or a pharmaceutically acceptable salt thereof, or the pharmaceutical composition of embodiment 1.18.

Embodiment 1.21. A method of treating or preventing metastasis of a cancer in a subject in need thereof, the method comprising administering to the subject an effective amount of the compound of any one of embodiments 1.1-1.17 or a pharmaceutically acceptable salt thereof, or the pharmaceutical composition of embodiment 1.18.

Embodiment 1.22. A method of sensitizing cancer for chemotherapy in a subject in need thereof, the method comprising administering to the subject an effective amount of the compound of any one of embodiments 1.1-1.17 or a pharmaceutically acceptable salt thereof, or the pharmaceutical composition of embodiment 1.18.

Embodiment 1.23. The method of any one of embodiments 1.19-1.22, further comprising administering to the subject an effective amount of a second anti-cancer therapy.

Embodiment 1.24. The method of embodiment 1.23, wherein the second anti-cancer therapy is a chemotherapeutic agent, a receptor tyrosine kinase inhibitor, or a therapeutic antibody.

Embodiment 1.25. The method of any one of embodiments 1.19-1.24, wherein the cancer is a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, sarcoma, gastrointestinal stromal tumor, peripheral nerve sheath tumor, myeloma, and/or endometrial cancer.

Embodiment 1.26. A method of treating or preventing type 2 diabetes in a subject in need thereof, the method comprising administering to the subject an effective amount of the compound of any one of embodiments 1.1-1.17 or a pharmaceutically acceptable salt thereof, or the pharmaceutical composition of embodiment 1.18.

Embodiment 1.27. A method of treating or preventing a metabolic disease in a subject in need thereof, the method comprising administering to the subject an effective amount of the compound of any one of embodiments 1.1-1.17 or a pharmaceutically acceptable salt thereof, or the pharmaceutical composition of embodiment 1.18.

Embodiment 1.28. A method of inhibiting an aldehyde dehydrogenase in a subject in need thereof, comprising administering to the subject an effective amount of the compound of any one of embodiments 1.1-1.17 or a pharmaceutically acceptable salt thereof, or the pharmaceutical composition of embodiment 1.18.

Embodiment 1.29. A method of treating a disease or disorder associated with aldehyde dehydrogenase, preferably, a disease or disorder associated with aldehyde dehydrogenase isoform 1a3 (ALDH1a3) and/or (ALDH1a2) in a subject in need thereof, comprising administering to the subject an effective amount of the compound of any one of embodiments 1.1-1.17, or a pharmaceutically acceptable salt thereof, or the pharmaceutical composition of embodiment 1.18.

Embodiment 1.30. The method of embodiment 1.27, wherein the disease or disorder is a proliferative disease or disorder or a metabolic disease or disorder.

Embodiment 1.31. A method of treating an endothelial cell or smooth muscle cell disease or disorder, such as pulmonary arterial hypertension or neointimal hyperplasia in a subject in need thereof, comprising administering to the subject an effective amount of the compound of any one of embodiments 1.1-1.17 or a pharmaceutically acceptable salt thereof, or the pharmaceutical composition of embodiment 1.18.

Embodiment 1.32. A method of antagonizing the retinoid pathway in a subject in need thereof, the method comprising administering to the subject an effective amount of the compound of any one of embodiments 1.1-1.17 or a pharmaceutically acceptable salt thereof, or the pharmaceutical composition of embodiment 1.18.

Embodiment 1.33. A method of treating a cancer in a subject in need thereof, the method comprising administering to the subject a therapeutically effective amount of the compound of any one of embodiments 1.1-1.17 or a pharmaceutically acceptable salt thereof, or the pharmaceutical composition of embodiment 1.18, in combination with an immunotherapy.

Embodiment 1.34. The method of embodiment 1.33, wherein the immunotherapy comprises administering to the subject an anti-PD-1 antibody, anti-PD-L1 antibody, anti-CTLA4 antibody, IL-2, autologous T cell therapy, bispecific antibody therapy, anti-TGFβ antibody, a JAK/STAT inhibitor, or any combination thereof.

Embodiment 1.35. The method of embodiment 1.33 or 1.34, wherein the cancer is a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, sarcoma, gastrointestinal stromal tumor, peripheral nerve sheath tumor, myeloma, and/or endometrial cancer.

Embodiment 1.36. A method of treating a cancer in a subject in need thereof, wherein the cancer is unresponsive to one or more immunotherapy or the subject has developed resistance to one or more immunotherapy, the method comprising administering to the subject a therapeutically effective amount of the compound of any one of embodiments 1.1-1.17 or a pharmaceutically acceptable salt thereof, or the pharmaceutical composition of claim 1.18, and optionally administering to the subject an immunotherapy.

Embodiment 1.37. The method of embodiment 36, wherein the cancer is unresponsive to treatment with anti-PD-1 or anti-PD-L1 antibodies.

Embodiment 1.38. The method of embodiment 36, wherein the subject has developed resistance to anti-PD-1 or anti-PD-L1 antibodies-based treatment.

Embodiment 1.39. The method of any one of embodiments 1.36-1.38, comprising administering to the subject an anti-PD-1 antibody, anti-PD-L1 antibody, anti-CTLA4 antibody, IL-2, autologous T cell therapy, bispecific antibody therapy, anti-TGFβ antibody, a JAK/STAT inhibitor, or any combination thereof.

Embodiment 1.40. The method of any one of embodiments 1.36-1.39, wherein the cancer is a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, sarcoma, gastrointestinal stromal tumor, peripheral nerve sheath tumor, myeloma, and/or endometrial cancer.

Embodiment 1.41. A method of treating a disease or disorder associated with retinoid pathway activation in a subject in need thereof, the method comprising administering to the subject a therapeutically effective amount of the compound of any one of embodiments 1.1-1.17 or a pharmaceutical salt thereof, or the pharmaceutical composition of embodiment 1.18.

Embodiment 1.42 The method of embodiment 1.41, wherein the disease or disorder is associated with immune tolerance, induction of T_{reg} cells and/or M2 macrophages, and/or effector T cell suppression.

Embodiment 1.43. The method of embodiment 1.41 or 1.42, wherein the disease or disorder is cancer.

Embodiment 1.44. The method of embodiment 1.43, wherein the cancer is a breast cancer, colorectal cancer, kidney cancer, ovarian cancer, gastric cancer, thyroid cancer, urothelial cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, hematologic cancer, sarcoma, gastrointestinal stromal tumor, peripheral nerve sheath tumor, myeloma, and/or endometrial cancer.

Embodiment 1.45. The method of any one of embodiments 1.41-1.44, further comprising administering to the subject an immunotherapy (e.g., an immune checkpoint inhibitor).

Embodiment 1.46. The method of embodiment 1.45, wherein administering the immunotherapy comprises administering to the subject an anti-PD-1 antibody, anti-PD-L1 antibody, anti-CTLA4 antibody, anti-LAG-3, anti-TIGIT, IL-2, autologous T cell therapy, bispecific antibody therapy, anti-TGFβ antibody, a JAK/STAT inhibitor, or any combination thereof.

Embodiment 1.47. A method of male contraception in a subject in need thereof, the method comprising administering to the subject an effective amount of the compound of any one of embodiments 1.1-1.17 or a pharmaceutically acceptable salt thereof, or the pharmaceutical composition of embodiment 1.18.

Embodiment 1.48. A method of inhibiting T_{reg} cell and/or M2 macrophage formation in a subject in need thereof, the method comprising administering to the subject an effective amount of the compound of any one of embodiments 1.1-1.17 or a pharmaceutical salt thereof, or the pharmaceutical composition of embodiment 1.18.

Embodiment 1.49. The method of embodiment 1.48, wherein the subject is characterized as having a cancer unresponsive to one or more immunotherapy or the subject has developed resistance to one or more immunotherapy.

Embodiment 1.50. The method of embodiment 1.28, wherein ALDH1a2 is selectively inhibited.

### FURTHER EMBODIMENTS

Embodiment 2.1. A compound of Formula (T)

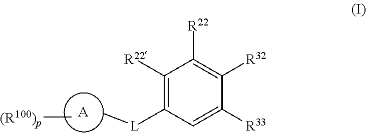

or a pharmaceutically acceptable salt or deuterated form thereof,
wherein:
   ring A is a heterocyclyl, heteroaryl, or aryl;
L is —NH—, —C(NR^{A})—NH—, —C(O)—NH—, —C(O)—N(R^{A})-alkylene-O—, -(alkylene)-N(R^{A})-alkylene-O—, —S(O)NH—, —S(O)₂NH—, —S(O)—, or —S(O)₂—;
   R²² is halo, —CO—N(R^{A})₂, —CN, —C₁₋₆ alkyl, —C₁₋₆ alkyl-CN, —C₁₋₆ haloalkyl, or carbocyclyl;
   R^{22′} is H, —CN, halo, —C₁₋₆ alkyl, —C₁₋₆haloalkyl, —C₁₋₆ alkyl-CN, carbocyclyl, or —C₁₋₆ alkyl-carbocyclyl; or
   R²² and R^{22′} are joined to form a heteroaryl, carbocyclyl, or heterocyclyl, each of which may be substituted with one or more halo;
   R³² and R³³ are joined to form a heterocyclyl and substituted with oxo; and
wherein the heterocyclyl may be further optionally substituted with one or more R¹⁰¹;
   p is 0, 1, or 2;
   each R¹⁰⁰ is independently halo, —C₁₋₆ alkyl, —C₁₋₆ alkylene-carbocyclyl, —C₁₋₆ alkylene-heterocyclyl, —C₁₋₆ haloalkyl, or aryl, wherein aryl is optionally substituted with 1 or 2 R¹⁰¹;
   each R¹⁰¹ is independently halo, —CO—N(R^{A})₂, —CN, hydroxy, —C₁₋₆ alkyl, or —C₁₋₆ haloalkyl; and
R^{A} is H, —C₁₋₆ alkyl, or aryl,
   wherein when L is —C(O)—NH— or —S(O)₂NH—, and A is aryl, then p is 1 or 2 and at least one 1 R¹⁰⁰ is aryl optionally substituted with one or more R¹⁰¹, and wherein the compound is not

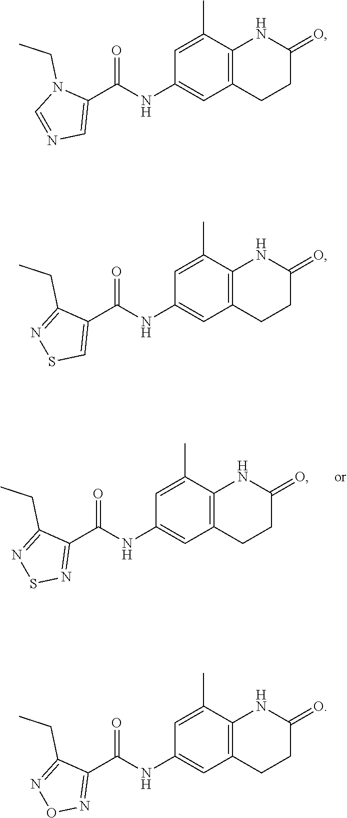 [SMILES: CCn1cncc1C(=O)Nc1cc(C)c2c(c1)CCC(=O)N2 ; CCc1nscc1C(=O)Nc1cc(C)c2c(c1)CCC(=O)N2 ; CCc1nsnc1C(=O)Nc1cc(C)c2c(c1)CCC(=O)N2 ; CCc1nonc1C(=O)Nc1cc(C)c2c(c1)CCC(=O)N2]

Embodiment 2.2. The compound of Embodiment 2.1, wherein the compound of Formula (I) is a compound of Formula (II):

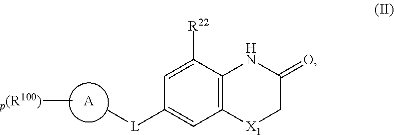

or a pharmaceutically acceptable salt or deuterated form thereof,
wherein:
X₁ is —S—, —S(O)₂—, —N(R^{A})— or —CH₂—;
ring A is heteroaryl or aryl;
L is —C(O)—NH—, —C(NR^{A})—NH—, —C(O)—N(R^{A})-alkylene-O—, or -(alkylene)-N(R^{A})-alkylene-O—;
R²² is halo, —CN, —C₁₋₆ alkyl, —C₁₋₆ alkyl-CN, —C₁₋₆ haloalkyl, -carbocyclyl, or —C₁₋₆ alkyl-carbocyclyl;
p is 0, 1, or 2;
each R¹⁰⁰ is independently halo, —C₁₋₆ alkyl, —C₁₋₆ alkylene-carbocyclyl, —C₁₋₆ alkylene-heterocyclyl, —C₁₋₆ haloalkyl, or aryl, wherein aryl is optionally substituted with 1 or 2 R¹⁰¹;
each R¹⁰¹ is independently halo, —CN, hydroxy, or —C₁₋₆ alkyl; and
R^{A} is H or —C₁₋₆ alkyl.

Embodiment 2.3. The compound of Embodiment 2.1, wherein the compound of Formula (I) is a compound of Formula (IIIA):

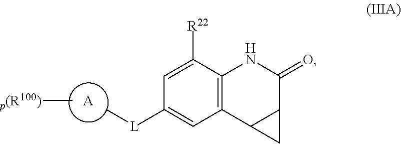

or a pharmaceutically acceptable salt or deuterated form thereof,
wherein:
ring A is heteroaryl or aryl;
L is —C(O)—NH—, —C(NR^{A})—NH—, —C(O)—N(R^{A})-alkylene-O—, or -(alkylene)-N(R^{A})-alkylene-O—;
R²² is halo, —CN, —C₁₋₆ alkyl, —C₁₋₆ alkyl-CN, —C₁₋₆ haloalkyl, -carbocyclyl, or —C₁₋₆ alkyl-carbocyclyl;
p is 0, 1, or 2;
each R¹⁰¹ is independently halo, —C₁₋₆ alkyl, —C₁₋₆alkylene-carbocyclyl, —C₁₋₆ alkylene-heterocyclyl, —C₁₋₆ haloalkyl, or aryl, wherein aryl is optionally substituted with 1 or 2 R¹⁰¹;
each R¹⁰¹ is independently halo, —CN, hydroxy, or —C₁₋₆ alkyl; and
R^{A} is H or —C₁₋₆ alkyl.

Embodiment 2.4. The compound of Embodiment 2.1, 2.2 or 2.3, wherein L is —C(O)—NH—, —C(O)—N(R^{A})-alkylene-O—, or -(alkylene)-N(R^{A})-alkylene-O—.

Embodiment 2.5. The compound of Embodiment 2.1, 2.2 or 2.3, wherein L is —C(O)—NH-Embodiment 2.6. The compound of any one of Embodiments 2.1-2.4, wherein ring A is a 5 or 6-membered heteroaryl, phenyl, a 5,6-bicyclic heteroaryl, a 5,6-bicyclic heterocyclyl, a 6,6-bicyclic heterocyclyl, a 6,6-bicyclic heteroaryl, or a 3-8 membered heterocyclyl.

Embodiment 2.7. The compound of Embodiment 2.6, wherein ring A is a 5-membered heteroaryl, 6-membered heteroaryl, or phenyl.

Embodiment 2.8. The compound of any one of Embodiments 2.1-2.7, wherein ring A is pyridyl, phenyl, thiazolyl, isothiazolyl, imidazolyl, oxazolyl, thiadiazolyl, or isoxazolyl.

Embodiment 2.9. The compound of any one of Embodiments 2.1-2.8, wherein ring A is

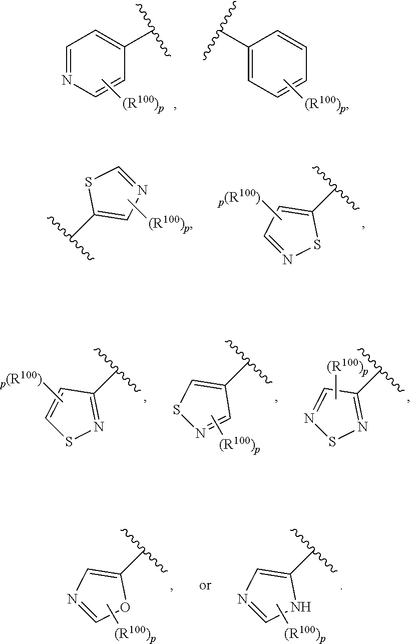

Embodiment 2.10. The compound of any one of Embodiments 2.1-2.9, wherein ring A-(R¹⁰⁰)ₚ is:

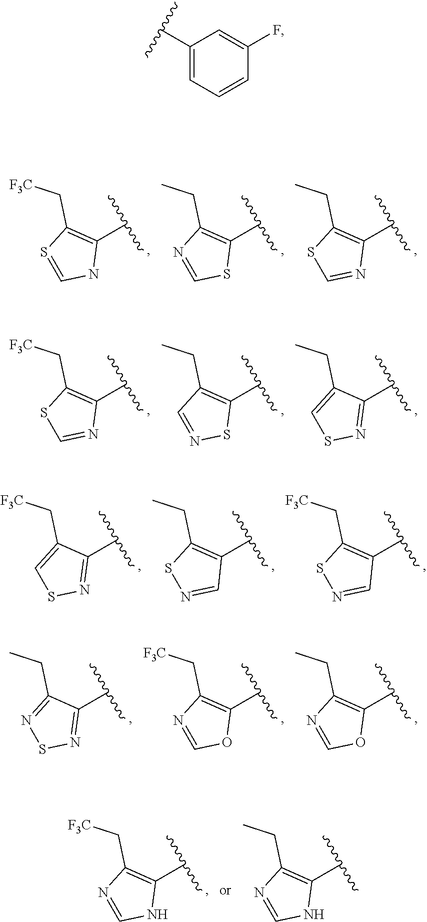

Embodiment 2.11. The compound of any one of Embodiments 2.1-2.9, wherein ring A-(R¹⁰⁰)ₚ is:

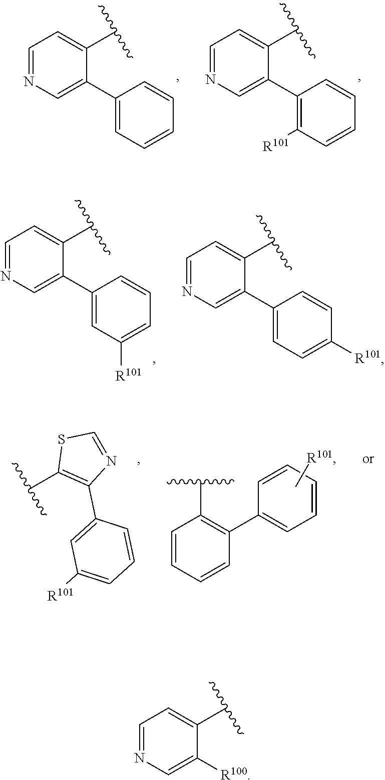

Embodiment 2.12. The compound of Embodiment 2.1, wherein the compound of Formula (I) is a compound of Formula (IV):

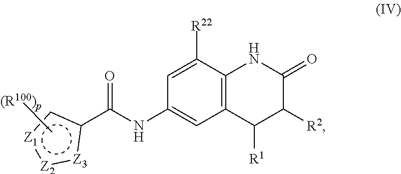

or a pharmaceutically acceptable salt or deuterated form thereof,
wherein:
R¹ is H and R² is H, or R¹ and R² are joined to form a carbocyclyl;
R²² is halo, —CN, —C₁₋₆ alkyl, —C₁₋₆ alkyl-CN, —C₁₋₆ haloalkyl, carbocyclyl, or —C₁₋₆ alkyl-carbocyclyl;
p is 0, 1, or 2;
each R¹⁰⁰ is independently halo, —CN, hydroxy, —C₁₋₆ alkylene-carbocyclyl, —C₁₋₆ alkyl, —C₁₋₆ haloalkyl, or carbocyclyl;
Z₁, Z₂, and Z₃ are independently selected from CH, S, N, NH, and O; and
dashed line represents a double or is absent to provide an aromatic ring.

Embodiment 2.13. The compound of Embodiment 2.12, wherein:

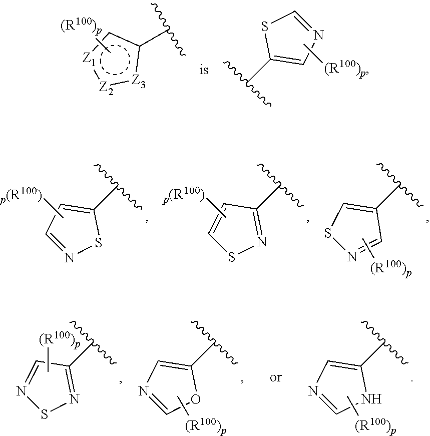

Embodiment 2.14. The compound of Embodiment 2.13 wherein:

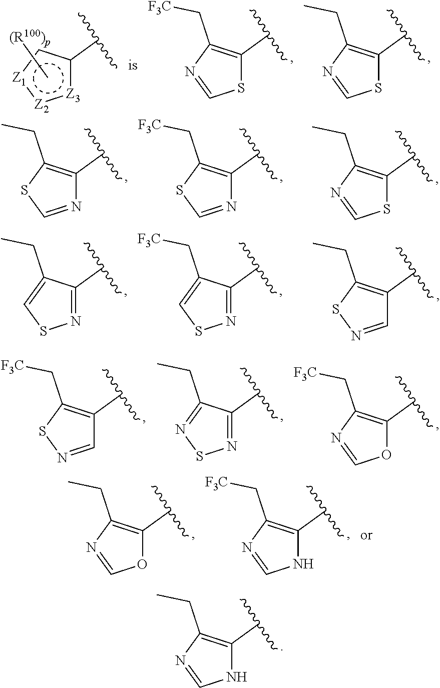

Embodiment 2.15. The compound of any one of Embodiments 2.1-2.14, wherein each R¹⁰⁰ is independently halo, —CN, —C₁₋₆ haloalkyl, —C₁₋₆alkylene-carbocyclyl, —C₁₋₆ alkyl, carbocyclyl, aryl, or heteroaryl, wherein aryl or heteroaryl is optionally substituted with 1 or 2 R¹⁰¹.

Embodiment 2.16. The compound of any one of Embodiments 2.1-2.9 or 2.15, wherein p is 1 and R¹⁰⁰ is aryl, wherein aryl is optionally substituted with 1 or 2 R¹⁰¹.

Embodiment 2.17. The compound of any one of Embodiments 2.1-2.9 and 2.16, wherein the compound of Formula (I) is a compound of Formula (V):

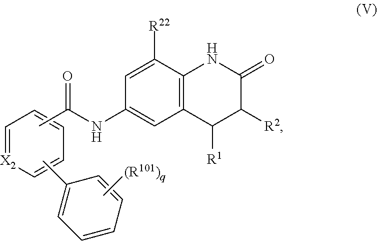

or a pharmaceutically acceptable salt or deuterated form thereof,
wherein:
R¹ is H and R² is H, or R¹ and R² are joined to form a carbocyclyl;
R²² is halo, —CN, —C₁₋₆ alkyl, —C₁₋₆ alkyl-CN, —C₁₋₆ haloalkyl, carbocyclyl, or —C₁₋₆ alkyl-carbocyclyl;
q is 0, 1, or 2;
each R¹⁰¹ is independently halo, —CN, hydroxy, —C₁₋₆ alkyl, or —C₁₋₆ haloalkyl; and
X₂ is —CH— or —N—.

Embodiment 2.18. The compound of any one of Embodiments 2.1-2.9 or 2.16, wherein

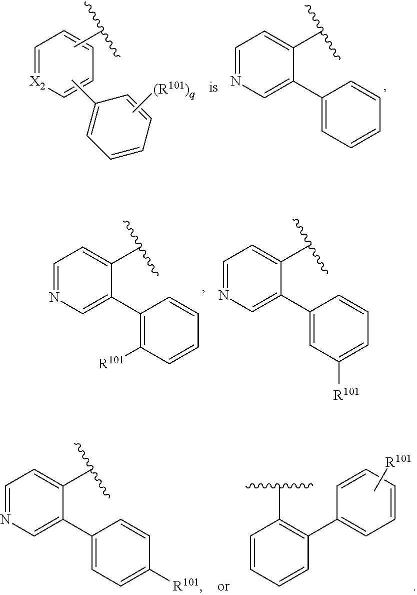

Embodiment 2.19. The compound of any one of Embodiments 2.1-2.9, 2.11, and 2.18, wherein R¹⁰¹ is fluoro, hydroxy, —CN, or methyl.

Embodiment 2.20. The compound of any one of Embodiments 2.1-2.19, wherein R²² is halo, —CN, —C₁₋₆ alkyl, or C₁₋₆ haloalkyl.

Embodiment 2.21. The compound of Embodiment 2.20, wherein R^{u} is halo or —C₁₋₆ alkyl.

Embodiment 2.22. The compound of Embodiment 2.21, wherein R²² is methyl or chloro.

Embodiment 2.23. The compound of Embodiment 2.1, wherein R² is —H, halo, —C₁₋₆ alkyl, or —C₁₋₆ haloalkyl.

Embodiment 2.24. The compound of Embodiment 2.1, wherein R^{22′} is —H, —F, or —Cl.

Embodiment 2.25. The compound of Embodiment 2.1, wherein R^{22′} is —H.

Embodiment 2.26. A compound selected from the group consisting of:

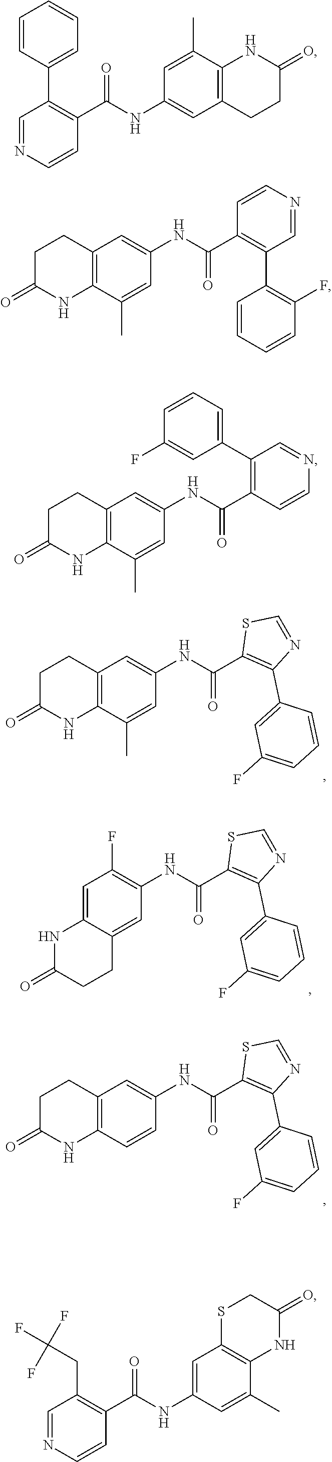

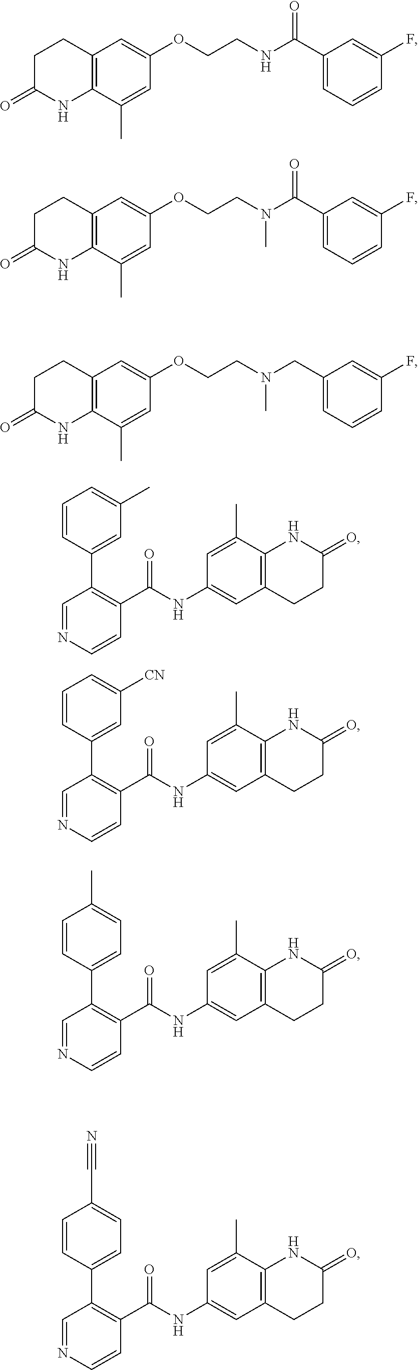

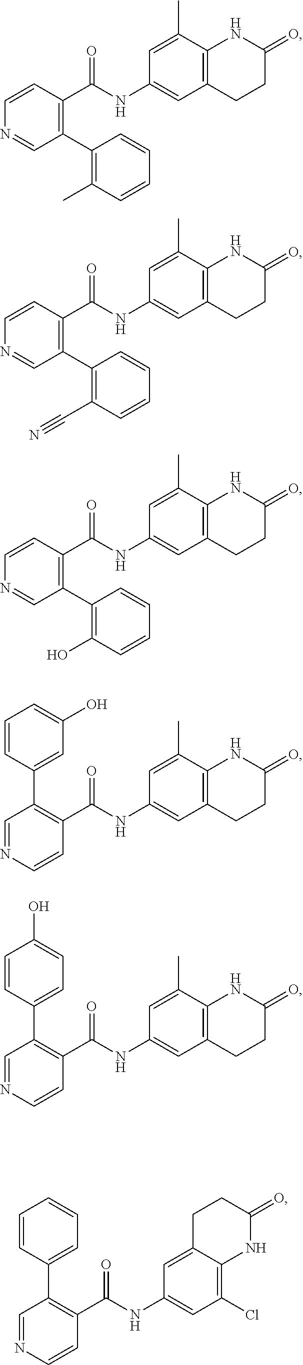

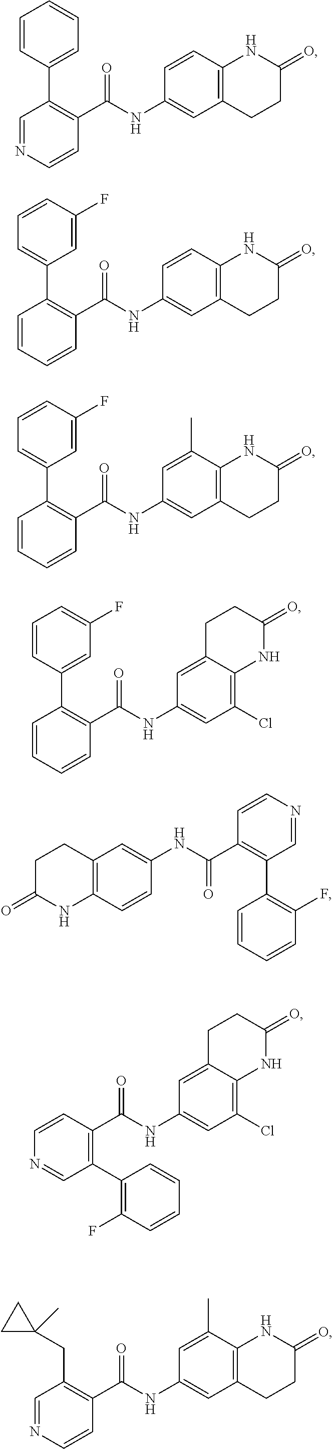

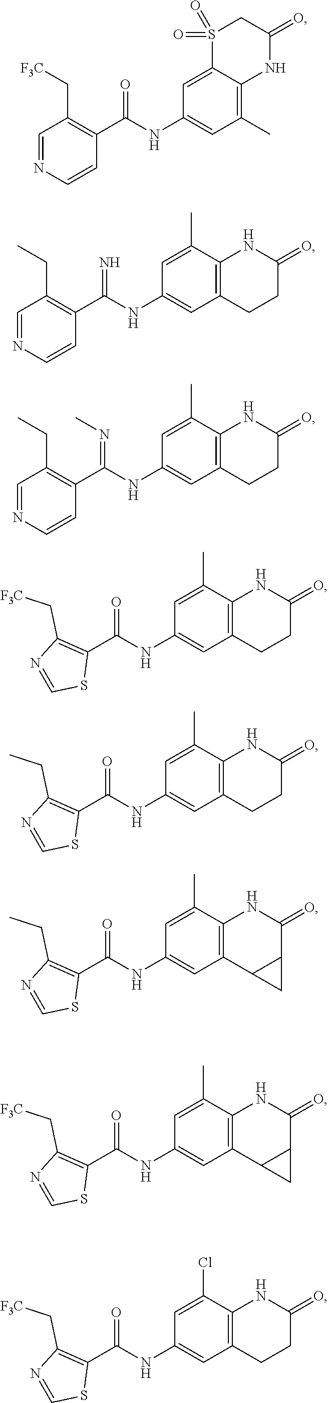

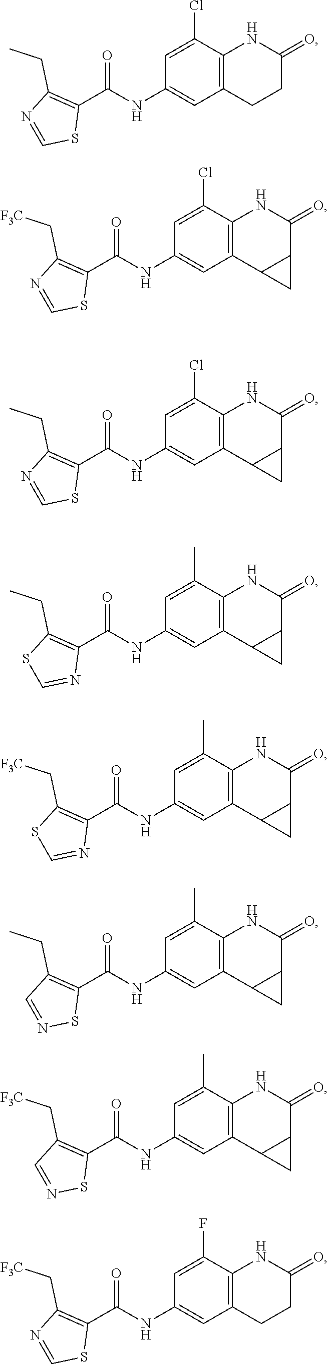

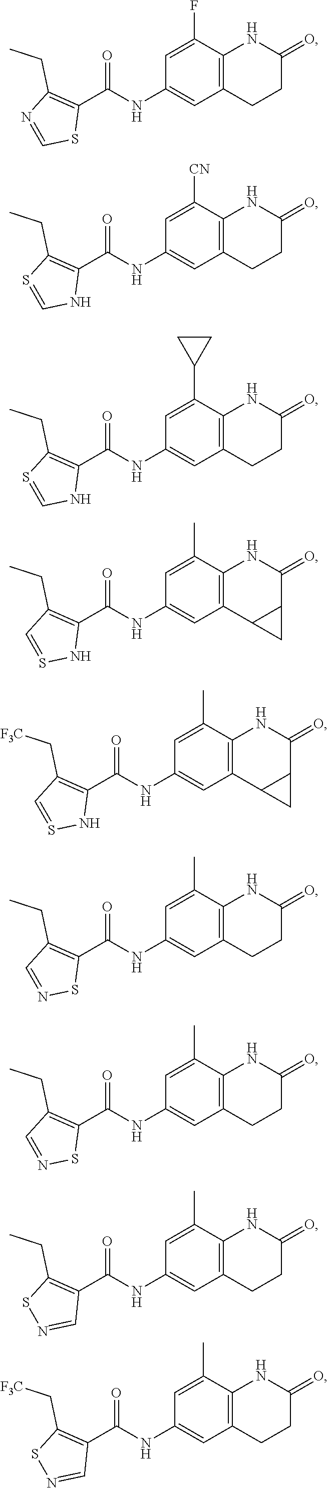

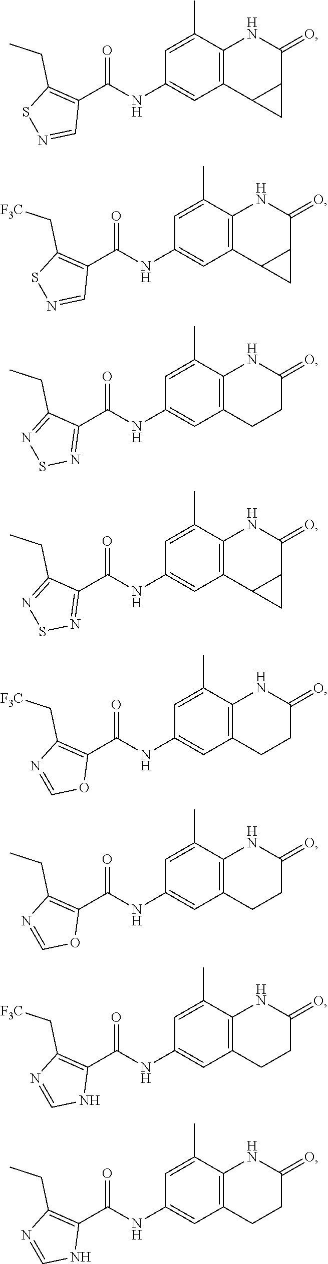

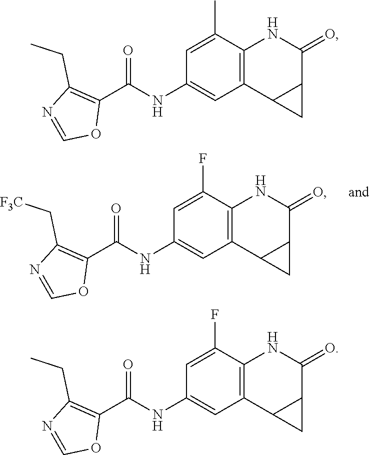 [SMILES: CCc1ncoc1C(=O)Nc1cc(C)c2c(c1)C1CC1C(=O)N2 ; O=C(Nc1cc(F)c2c(c1)C1CC1C(=O)N2)c1ocnc1CC(F)(F)F ; "and" ; CCc1ncoc1C(=O)Nc1cc(F)c2c(c1)C1CC1C(=O)N2]

Embodiment 2.27. A pharmaceutical composition comprising the compound of any one of Embodiments 2.1-2.26, or a pharmaceutically acceptable salt thereof, and a pharmaceutically acceptable carrier.

Embodiment 2.28. A method of treating cancer in a subject in need thereof, the method comprising administering to the subject a therapeutically effective amount of the compound of any one of Embodiments 2.1-2.26 or a pharmaceutically acceptable salt thereof, or the pharmaceutical composition of Embodiment 2.27.

Embodiment 29. A method of treating metastatic cancer or chemoresistant cancer in a subject in need thereof, the method comprising administering to the subject a therapeutically effective amount of the compound of any one of Embodiments 2.1-2.26 or a pharmaceutically acceptable salt thereof, or the pharmaceutical composition of Embodiment 2.27.

Embodiment 2.30. A method of treating or preventing metastasis of a cancer in a subject in need thereof, the method comprising administering to the subject an effective amount of the compound of any one of Embodiments 2.1-2.26 or a pharmaceutically acceptable salt thereof, or the pharmaceutical composition of Embodiment 2.27.

Embodiment 2.31. A method of sensitizing cancer for chemotherapy in a subject in need thereof, the method comprising administering to the subject an effective amount of the compound of any one of Embodiments 2.1-2.26 or a pharmaceutically acceptable salt thereof, or the pharmaceutical composition of Embodiment 2.27.

Embodiment 2.32. The method of any one of Embodiments 2.38-2.31, further comprising administering to the subject an effective amount of a second anti-cancer therapy.

Embodiment 2.33. The method of Embodiment 2.32, wherein the second anti-cancer therapy is a chemotherapeutic agent, a receptor tyrosine kinase inhibitor, or a therapeutic antibody.

Embodiment 2.34. The method of any one of Embodiments 2.28-2.33, wherein the cancer is a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, sarcoma, gastrointestinal stromal tumor, peripheral nerve sheath tumor, myeloma, and/or endometrial cancer.

Embodiment 2.35. A method of treating or preventing type 2 diabetes in a subject in need thereof, the method comprising administering to the subject an effective amount of the compound of any one of Embodiments 2.1-2.26 or a pharmaceutically acceptable salt thereof, or the pharmaceutical composition of Embodiment 2.27.

Embodiment 2.36. A method of treating or preventing a metabolic disease in a subject in need thereof, the method comprising administering to the subject an effective amount of the compound of any one of Embodiments 2.1-2.26 or a pharmaceutically acceptable salt thereof, or the pharmaceutical composition of Embodiment 2.27.

Embodiment 2.37. A method of inhibiting an aldehyde dehydrogenase in a subject in need thereof, comprising administering to the subject an effective amount of the compound of any one of Embodiments 2.1-2.26 or a pharmaceutically acceptable salt thereof, or the pharmaceutical composition of Embodiment 2.27.

Embodiment 2.38. A method of treating a disease or disorder associated with aldehyde dehydrogenase, preferably, a disease or disorder associated with aldehyde dehydrogenase isoform 1a3 (ALDH1a3) and/or (ALDH1a2) in a subject in need thereof, comprising administering to the subject an effective amount of the compound of any one of Embodiments 2.1-2.26, or a pharmaceutically acceptable salt thereof, or the pharmaceutical composition of Embodiment 2.27.

Embodiment 2.39. The method of Embodiment 2.38, wherein the disease or disorder is a proliferative disease or disorder or a metabolic disease or disorder.

Embodiment 2.40. A method of treating an endothelial cell or smooth muscle cell disease or disorder, such as pulmonary arterial hypertension or neointimal hyperplasia in a subject in need thereof, comprising administering to the subject an effective amount of the compound of any one of Embodiments 2.1-2.26 or a pharmaceutically acceptable salt thereof, or the pharmaceutical composition of Embodiment 2.27.

Embodiment 2.41. A method of antagonizing the retinoid pathway in a subject in need thereof, the method comprising administering to the subject an effective amount of the compound of any one of Embodiments 2.1-2.26 or a pharmaceutically acceptable salt thereof, or the pharmaceutical composition of Embodiment 2.27.

Embodiment 2.42. A method of treating a cancer in a subject in need thereof, the method comprising administering to the subject a therapeutically effective amount of the compound of any one of Embodiments 2.1-2.26 or a pharmaceutically acceptable salt thereof, or the pharmaceutical composition of Embodiment 2.27, in combination with an immunotherapy.

Embodiment 2.43. The method of Embodiment 2.42, wherein the immunotherapy comprises administering to the subject an anti-PD-1 antibody, anti-PD-L1 antibody, anti-CTLA4 antibody, IL-2, autologous T cell therapy, bispecific antibody therapy, anti-TGFβ antibody, a JAK/STAT inhibitor, or any combination thereof.

Embodiment 2.44. The method of Embodiment 2.42 or 2.43, wherein the cancer is a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, sarcoma, gastrointestinal stromal tumor, peripheral nerve sheath tumor, myeloma, and/or endometrial cancer.

Embodiment 2.45. A method of treating a cancer in a subject in need thereof, wherein the cancer is unresponsive to one or more immunotherapy or the subject has developed resistance to one or more immunotherapy, the method comprising administering to the subject a therapeutically effective amount of the compound of any one of Embodiments 2.1-2.26 or a pharmaceutically acceptable salt thereof, or the pharmaceutical composition of Embodiment 2.27, and optionally administering to the subject an immunotherapy.

Embodiment 2.46. The method of Embodiment 2.45, wherein the cancer is unresponsive to treatment with anti-PD-1 or anti-PD-L1 antibodies.

Embodiment 2.47. The method of Embodiment 2.45, wherein the subject has developed resistance to anti-PD-1 or anti-PD-L1 antibodies-based treatment.

Embodiment 2.48. The method of any one of Embodiments 2.45-2.47, comprising administering to the subject an anti-PD-1 antibody, anti-PD-L1 antibody, anti-CTLA4 antibody, IL-2, autologous T cell therapy, bispecific antibody therapy, anti-TGFβ antibody, a JAK/STAT inhibitor, or any combination thereof.

Embodiment 2.49. The method of any one of Embodiments 2.45-2.48, wherein the cancer is a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, sarcoma, gastrointestinal stromal tumor, peripheral nerve sheath tumor, myeloma, and/or endometrial cancer.

Embodiment 2.50. A method of treating a disease or disorder associated with retinoid pathway activation in a subject in need thereof, the method comprising administering to the subject a therapeutically effective amount of the compound of any one of Embodiments 1-26 or a pharmaceutical salt thereof, or the pharmaceutical composition of Embodiment 27.

Embodiment 2.51. The method of Embodiment 2.50, wherein the disease or disorder is associated with immune tolerance, induction of T_{reg} cells and/or M2 macrophages, and/or effector T cell suppression.

Embodiment 2.52. The method of Embodiment 2.50 or 2.51, wherein the disease or disorder is cancer.

Embodiment 2.53. The method of Embodiment 2.52, wherein the cancer is a breast cancer, colorectal cancer, kidney cancer, ovarian cancer, gastric cancer, thyroid cancer, urothelial cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, hematologic cancer, sarcoma, gastrointestinal stromal tumor, peripheral nerve sheath tumor, myeloma, and/or endometrial cancer.

Embodiment 2.54. The method of any one of Embodiments 2.50-2.53, further comprising administering to the subject an immunotherapy (e.g., an immune checkpoint inhibitor).

Embodiment 2.55. The method of Embodiment 2.54, wherein administering the immunotherapy comprises administering to the subject an anti-PD-1 antibody, anti-PD-L1 antibody, anti-CTLA4 antibody, anti-LAG-3, anti-TIGIT, IL-2, autologous T cell therapy, bispecific antibody therapy, anti-TGFβ antibody, a JAK/STAT inhibitor, or any combination thereof.

Embodiment 2.56. A method of male contraception in a subject in need thereof, the method comprising administering to the subject an effective amount of the compound of any one of Embodiments 2.1-2.26 or a pharmaceutically acceptable salt thereof, or the pharmaceutical composition of Embodiment 2.27.

Embodiment 2.57. A method of inhibiting T_{reg} cell and/or M2 macrophage formation in a subject in need thereof, the method comprising administering to the subject an effective amount of the compound of any one of Embodiments 2.1-2.26 or a pharmaceutical salt thereof, or the pharmaceutical composition of Embodiment 2.27.

Embodiment 2.58. The method of Embodiment 2.57, wherein the subject is characterized as having a cancer unresponsive to one or more immunotherapy or the subject has developed resistance to one or more immunotherapy.

Embodiment 2.59. The method of Embodiment 2.38, wherein ALDH1a2 is selectively inhibited.

### EXAMPLES

The various starting materials, intermediates, and compounds of the embodiments can be isolated and purified where appropriate using conventional techniques such as precipitation, filtration, crystallization, evaporation, distillation, and chromatography. Characterization of these compounds can be performed using conventional methods such as by melting point, mass spectrum, nuclear magnetic resonance, and various other spectroscopic analyses. Exemplary embodiments of steps for performing the synthesis of products described herein are described in greater detail infra.

### Example 1: Synthesis of N-(8-methyl-2-oxo-1,2,3,4-tetrahydroquinolin-6-yl)-3-phenylisonicotinamide (Compound 1)

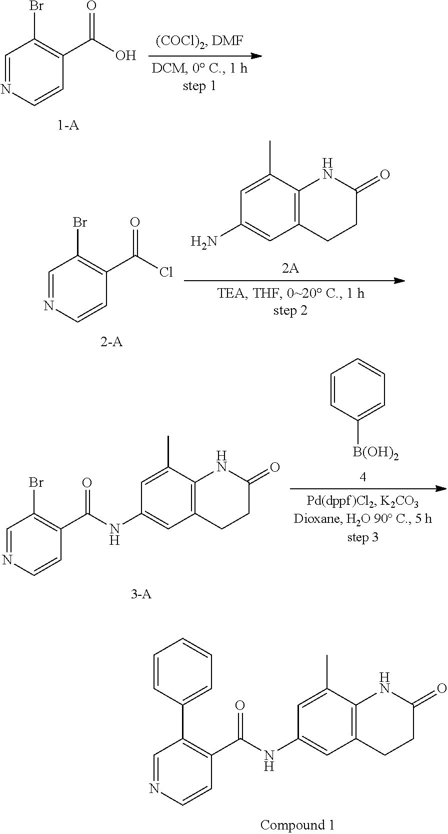 [SMILES: O=C(O)c1ccncc1Br ; O=C(Cl)c1ccncc1Br ; Cc1cc(N)cc2c1NC(=O)CC2 ; Cc1cc(NC(=O)c2ccncc2Br)cc2c1NC(=O)CC2 ; OB(O)c1ccccc1 ; Cc1cc(NC(=O)c2ccncc2-c2ccccc2)cc2c1NC(=O)CC2]

Step 1: To the mixture of 3-bromopyridine-4-carboxylic acid (500 mg, 2.48 mmol, 1 eq) and DMF (9.05 mg, 123.76 μmol, 9.52 μL, 0.05 eq) in DCM (10 mL) was added (COCl)₂ (628.33 mg, 4.95 mmol, 2 eq) drop-wise at 0° C. Then the mixture was stirred at 0° C. for 1 hr. The mixture was concentrated to get 3-bromopyridine-4-carbonyl chloride (550 mg, crude) as an oil.

Step 2: To the mixture of 6-amino-8-methyl-3,4-dihydro-1H-quinolin-2-one (395.67 mg, 2.25 mmol, 0.9 eq) and TEA (757.36 mg, 7.48 mmol, 1.04 mL, 3 eq) in THF (10 mL) was added 3-bromopyridine-4-carbonyl chloride (550 mg, 2.49 mmol, 1 eq) in DCM (2 mL) drop-wise at 0° C. The mixture was stirred at 20° C. for 1 hr under N₂. LC-MS showed 6-amino-8-methyl-3,4-dihydro-1H-quinolin-2-one was consumed completely and one main peak with desired m/z was detected. To the mixture was added water (10 ml). The aqueous phase was extracted with DCM (10 mL*4). The combined organic phase was washed with brine (10 mL), dried with anhydrous Na₂SO₄, filtered and concentrated in vacuum. 3-bromo-N-(8-methyl-2-oxo-3,4-dihydro-1H-quinolin-6-yl)pyridine-4-carboxamide (600 mg, 1.67 mmol, 66.77% yield) was obtained as a solid. (M+H)⁺: 360.0, 362.0. ¹H NMR (400 MHz, DMSO-d6) δ ppm 10.47 (s, 1H), 9.46 (s, 1H), 8.87 (s, 1H), 8.68 (d, J=4.8 Hz, 1H), 7.58 (d, J=4.8 Hz, 1H), 7.38 (s, 1H), 7.30 (d, J=1.6 Hz, 1H), 2.86 (t, J=6.8 Hz, 2H), 2.44 (t, J=6.8 Hz, 2H), 2.22 (s, 3H).

Step 4: To a solution of 3-bromo-N-(8-methyl-2-oxo-3,4-dihydro-1H-quinolin-6-yl)pyridine-4-carboxamide (100 mg, 277.62 umol, 1 eq) and phenylboronic acid (40.62 mg, 333.14 umol, 1.2 eq) in dioxane (4 mL) and H₂O (0.8 mL) was added Na₂CO₃ (58.85 mg, 555.24 umol, 2 eq) and cyclopentyl(diphenyl)phosphane; dichloropalladium; iron (20.31 mg, 27.76 umol, 0.1 eq). The mixture was stirred at 90° C. for 5 hr. LCMS showed the reaction was consumed completely. The mixture was filtered. The filtrate was concentrated in vacuum. The crude product was purified by column chromatography (SiO2, Ethyl acetate/MeOH=1/0 to 10/1). The product of N-(8-methyl-2-oxo-3,4-dihydro-1H-quinolin-6-yl)-3-phenyl-pyridine-4-carboxamide (27 mg, 74.03 μmol, 26.67% yield) was obtained as a solid. (M+H)+: 358.1. ¹H NMR (400 MHz, MeOD) δ ppm 8.70-8.75 (m, 2H), 7.62 (d, J=4.8 Hz, 1H), 7.52-7.55 (m, 2H), 7.42-7.47 (m, 2H), 7.17 (s, 1H), 7.09 (s, 1H), 2.90 (t, J=6.8 Hz, 2H), 2.55 (t, J=6.8 Hz, 2H), 2.22 (s, 3H).

### Example 2: Synthesis of 3-(2-fluorophenyl)-N-(8-methyl-2-oxo-1,2,3,4-tetrahydroquinolin-6-yl)isonicotinamide (Compound 2)

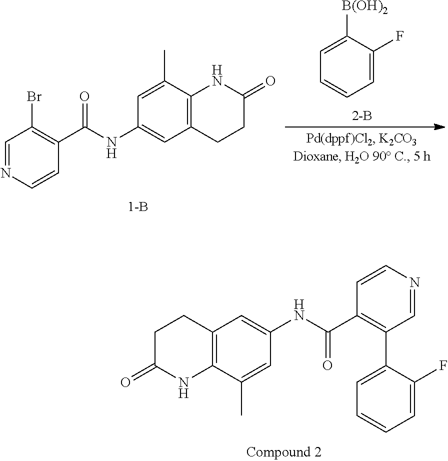 [SMILES: Cc1cc(NC(=O)c2ccncc2Br)cc2c1NC(=O)CC2 ; OB(O)c1ccccc1F ; Cc1cc(NC(=O)c2ccncc2-c2ccccc2F)cc2c1NC(=O)CC2]

Step 3: To a solution of 3-bromo-N-(8-methyl-2-oxo-3,4-dihydro-1H-quinolin-6-yl)pyridine-4-carboxamide (100 mg, 277.62 umol, 1 eq) and (2-fluorophenyl)boronic acid (46.61 mg, 333.14 umol, 1.2 eq) in dioxane (4 mL) and H₂O (0.8 mL) was added cyclopentyl(diphenyl)phosphane;dichloropalladium;iron (20.31 mg, 27.76 umol, 0.1 eq) and Na₂CO₃ (58.85 mg, 555.24 μmol, 2 eq). The mixture was stirred at 90° C. for 5 hr. The mixture was filtered. The filtrate was concentrated in vacuum. The residue was diluted with H₂O 15 mL and extracted with EtOAc 7 mL*3. The combined organic layers were washed with brine 3 mL*3, dried over Na₂SO₄, filtered and concentrated under reduced pressure to give a residue. The crude product was purified by column chromatography (SiO₂, Ethyl acetate/MeOH=1/0 to 10/1). The product of 3-(2-fluorophenyl)-N-(8-methyl-2-oxo-3,4-dihydro-1H-quinolin-6-yl)pyridine-4-carboxamide (12.5 mg, 31.97 μmol, 11.51% yield) was obtained as a solid. (M+H)⁺: 376.1. ¹H NMR (400 MHz, MeOD) δ ppm 8.72 (d, J=5.2 Hz, 1H), 8.66 (s, 1H), 7.68 (d, J=5.2 Hz, 1H), 7.41-7.50 (m, 2H), 7.24-7.31 (m, 1H), 7.09-7.21 (m, 3H), 2.89 (t, J=6.8 Hz, 2H), 2.54 (d, J=6.8 Hz, 2H), 2.22 (s, 3H).

### Example 3: Synthesis of 3-(3-fluorophenyl)-N-(8-methyl-2-oxo-1,2,3,4-tetrahydroquinolin-6-yl)isonicotinamide (Compound 3)

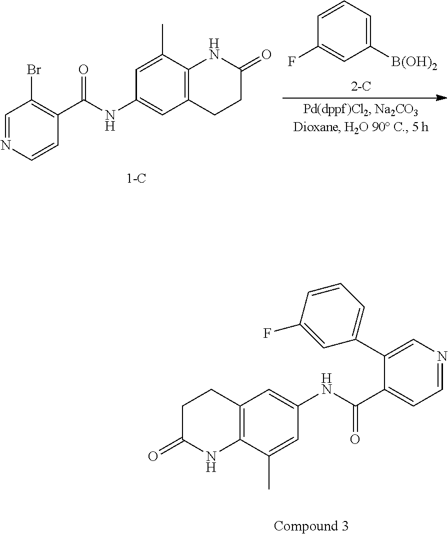

Step 1: To a solution of (3-fluorophenyl)boronic acid (46.61 mg, 333.14 umol, 1.2 eq) and 3-bromo-N-(8-methyl-2-oxo-3,4-dihydro-1H-quinolin-6-yl)pyridine-4-carboxamide (100 mg, 277.62 umol, 1 eq) in dioxane (4 mL) and H₂O (0.8 mL) was added Pd(dppf)Cl₂ (20.31 mg, 27.76 umol, 0.1 eq) and Na₂CO₃ (58.85 mg, 555.24 μmol, 2 eq). The mixture was stirred at 90° C. for 5 hr. LCMS showed the reaction was consumed completely. The mixture was filtered. The filtrate was concentrated in vacuum. The crude product was purified by column chromatography (SiO₂, Ethyl acetate/MeOH=1/0 to 10/1). The product of 3-(3-fluorophenyl)-N-(8-methyl-2-oxo-3,4-dihydro-1H-quinolin-6-yl)pyridine-4-carboxamide (39.7 mg, 101.53 μmol, 36.57% yield) was obtained as a solid. (M+H)⁺: 376.1. ¹H NMR (400 MHz, METHANOL-d4) δ ppm 8.66-8.73 (m, 2H), 7.62 (d, J=4.8 Hz, 1H), 7.42-7.49 (m, 1H), 7.26-7.34 (m, 2H), 7.07-7.19 (m, 3H), 2.91 (t, J=7.6 Hz, 2H) 2.56 (t, J=7.6 Hz, 2H), 2.23 (s, 3H).

### Example 4: Synthesis of 4-(3-fluorophenyl)-N-(8-methyl-2-oxo-1,2,3,4-tetrahydroquinolin-6-yl)thiazole-5-carboxamide (Compound 4)

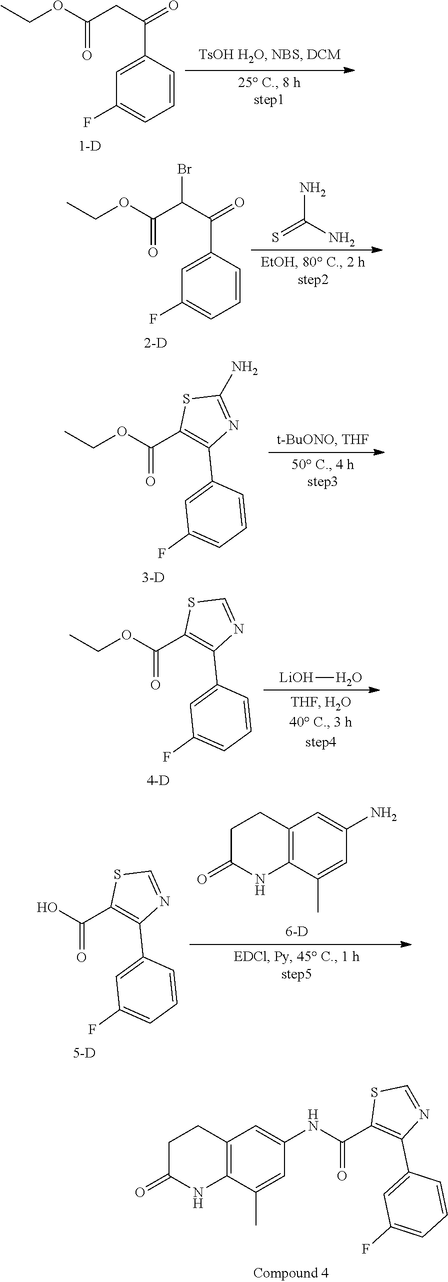

Step 1: To a solution of ethyl 3-(3-fluorophenyl)-3-oxo-propanoate (3 g, 14.27 mmol, 1 eq) in DCM (30 mL) was added 4-methylbenzenesulfonic acid, hydrate (542.96 mg, 2.85 mmol, 0.2 eq) and NBS (5.08 g, 28.54 mmol, 2 eq). The mixture was stirred at 25° C. for 8 hr. TLC indicated the reaction was consumed completely. The reaction mixture was diluted with H₂O 50 mL and extracted with EtOAc 50 mL (25 mL*2). The combined organic layers were dried over Na₂SO₄, filtered and the filtrate concentrated under reduced pressure to give a residue. The residue was purified by column chromatography (SiO₂, Petroleum ether/Ethyl acetate=50/1 to 10/1). The product of ethyl 2-bromo-3-(3-fluorophenyl)-3-oxo-propanoate (2 g, 6.23 mmol, 43.63% yield) was obtained as a oil. ¹H NMR (400 MHz, CHLOROFORM-d) δ ppm 7.78 (d, J=8 Hz, 1H), 7.68-7.72 (m, 1H), 7.45-7.56 (m, 1H), 7.32-7.34 (m, 1H), 5.60 (s, 1H), 4.31 (q, J=7.2 Hz, 2H), 1.23-1.31 (m, 3H).

Step 2: To a solution of ethyl 2-bromo-3-(3-fluorophenyl)-3-oxo-propanoate (2.39 g, 8.27 mmol, 1 eq) and thiourea (755.16 mg, 9.92 mmol, 1.2 eq) in EtOH (20 mL). The mixture was stirred at 80° C. for 2 hr. TLC indicated the reaction was consumed completely. The reaction mixture was concentrated under reduced pressure to give a residue. The residue was purified by column chromatography (SiO₂, Petroleum ether/Ethyl acetate=10/1 to 0/1). The product of ethyl 2-amino-4-(3-fluorophenyl)thiazole-5-carboxylate (1.2 g, 4.51 mmol, 54.51% yield) was obtained as an oil. ¹H NMR (400 MHz, CHLOROFORM-d) δ ppm 7.49 (d, J=7.8 Hz, 1H) 7.33-7.45 (m, 2H), 7.10 (d, J=8.4 Hz, 1H), 5.64 (br s, 2H), 4.22 (q, J=7.1 Hz, 2H), 1.26 (t, J=7.1 Hz, 3H).

Step 3: To a solution of ethyl 2-amino-4-(3-fluorophenyl)thiazole-5-carboxylate (840 mg, 3.15 mmol, 1 eq) in THF (10 mL) was added t-BuONO (422.87 mg, 4.10 mmol, 487.74 μL, 1.3 eq). The mixture was stirred at 50° C. for 4 hr. TLC indicated the reaction was consumed completely. The reaction mixture was concentrated under reduced pressure to give a residue. The residue was purified by column chromatography (SiO₂, Petroleum ether/Ethyl acetate=50/1 to 5/1). The product of ethyl 4-(3-fluorophenyl)thiazole-5-carboxylate (800 mg, 2.80 mmol, 88.82% yield) was obtained as a solid. ¹H NMR (400 MHz, CHLOROFORM-d) δ ppm 8.91 (s, 1H), 7.58 (d, J=7.6 Hz, 1H), 7.52-7.55 (m, 1H), 7.40-7.43 (m, 1H), 7.10-7.18 (m, 1H), 4.32 (q, J=7.2 Hz, 2H), 1.32 (t, J=7.2 Hz, 3H).

Step 4: To a solution of ethyl 4-(3-fluorophenyl)thiazole-5-carboxylate (800 mg, 3.18 mmol, 1 eq) in THF (10 mL) was added LiOH·H₂O (267.20 mg, 6.37 mmol, 2 eq) in H₂O (2 mL). The mixture was stirred at 40° C. for 3 hr. LC-MS showed the reaction was consumed completely. The reaction mixture was concentrated under reduced pressure to give a residue. The reaction mixture was diluted with H₂O 10 mL and extracted with EtOAc 10 mL. The aqueous layer was adjusted to pH=3 by addition of 1N HCl. The reaction mixture was filtered and the filter cake concentrated under reduced pressure to give a residue. The product of 4-(3-fluorophenyl)thiazole-5-carboxylic acid (700 mg, 3.14 mmol, 98.50% yield) was obtained as a solid. (M+H)⁺: 224.1. ¹H NMR (400 MHz, DMSO-d6) δ ppm 13.48 (s, 1H), 9.28 (s, 1H), 7.56-7.64 (m, 2H), 7.48 (td, J=8.0, 6.4 Hz, 1H), 7.27 (td, J=8.7, 2.4 Hz, 1H).

Step 5: To a solution of 4-(3-fluorophenyl)thiazole-5-carboxylic acid (50 mg, 223.99 umol, 1 eq) and 6-amino-8-methyl-3,4-dihydro-1H-quinolin-2-one (43.42 mg, 246.39 umol, 1.1 eq) in pyridine (1 mL) was added EDCI (64.41 mg, 335.99 umol, 1.5 eq). The mixture was stirred at 45° C. for 1 hr. LC-MS showed the reaction was consumed completely. The reaction mixture was concentrated under reduced pressure to give a residue. The residue was purified by prep-TLC (SiO₂, Ethyl acetate:MeOH=20:1). The product of 4-(3-fluorophenyl)-N-(8-methyl-2-oxo-3,4-dihydro-1H-quinolin-6-yl) thiazole-5-carboxamide (14.6 mg, 37.89 umol, 16.92% yield) was obtained as a solid. (M+H)⁺: 382.0. ¹H NMR (400 MHz, DMSO-d6) δ ppm 10.48 (s, 1H), 9.44 (s, 1H), 9.27 (s, 1H), 7.44-7.63 (m, 3H), 7.19-7.30 (m, 3H), 2.83 (br t, J=7.6 Hz, 2H), 2.40 (br t, J=7.6 Hz, 2H), 2.18 (s, 3H).

### Example 5: Synthesis of N-(7-fluoro-2-oxo-1,2,3,4-tetrahydroquinolin-6-yl)-4-(3-fluorophenyl)thiazole-5-carboxamide (Compound 5)

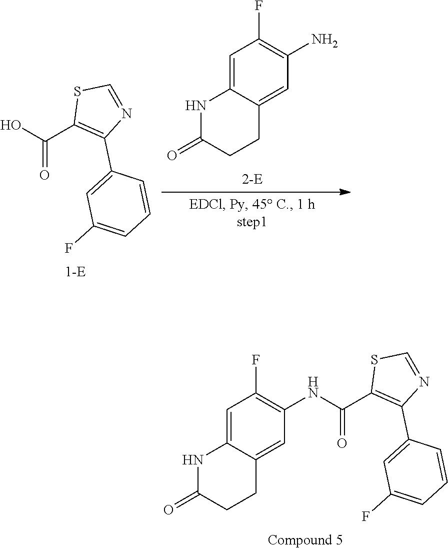

To a solution of 4-(3-fluorophenyl)thiazole-5-carboxylic acid (60 mg, 268.79 umol, 1 eq) and 6-amino-7-fluoro-3,4-dihydro-1H-quinolin-2-one (53.27 mg, 295.67 umol, 1.1 eq) in pyridine (1 mL) was added EDCI (77.29 mg, 403.18 umol, 1.5 eq). The mixture was stirred at 45° C. for 1 hr. LC-MS showed the reaction was consumed completely. The reaction mixture was concentrated under reduced pressure to give a residue. The residue was purified by prep-TLC (SiO₂, Ethyl acetate:MeOH=20:1). The product of 4-(3-fluorophenyl)-N-(8-methyl-2-oxo-3,4-dihydro-1H-quinolin-6-yl)thiazole-5-carboxamide (40.4 mg, 103.80 umol, 38.62% yield) was obtained as a solid. (M+H)⁺: 386.0. ¹H NMR (400 MHz, DMSO-d6) δ ppm 10.13 10.30 (m, 2H), 9.28 (s, 1H), 7.46-7.68 (m, 3H), 7.35 (br d, J=7.2 Hz, 1H), 7.24-7.32 (m, 1H), 6.72 (br d, J=11.6 Hz, 1H), 2.86 (br t, J=6.8 Hz, 2H), 2.43-2.48 (m, 2H).

### Example 6: Synthesis of 4-(3-fluorophenyl)-N-(2-oxo-1,2,3,4-tetrahydroquinolin-6-yl)thiazole-5-carboxamide (Compound 6)

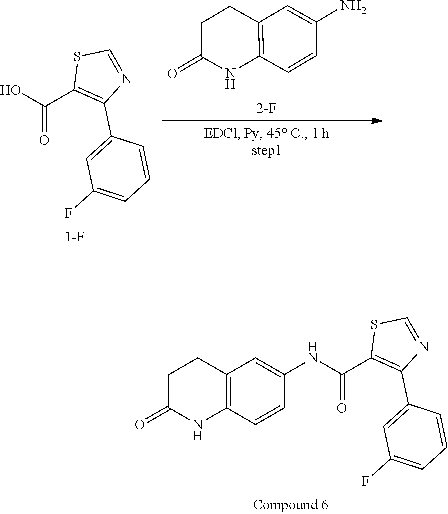

Step 1: To a solution of 4-(3-fluorophenyl)thiazole-5-carboxylic acid (60 mg, 268.79 umol, 1 eq) and 6-amino-3,4-dihydro-1H-quinolin-2-one (47.95 mg, 295.67 umol, 1.1 eq) in pyridine (1 mL) was added EDCI (77.29 mg, 403.19 umol, 1.5 eq). The mixture was stirred at 45° C. for 1 hr. LC-MS showed the reaction was consumed completely. The reaction mixture was concentrated under reduced pressure to give a residue. The residue was purified by prep-TLC (SiO₂, Ethyl acetate/MeOH=20:1). The product of 4-(3-fluorophenyl)-N-(8-methyl-2-oxo-3,4-dihydro-1H-quinolin-6-yl)thiazole-5-carboxamide (30 mg, 78.65 umol, 29.26% yield) was obtained as a solid. (M+H)⁺: 368.0. ¹H NMR (400 MHz, DMSO-d6) δ ppm 10.52 (s, 1H), 10.06 (s, 1H), 9.27 (s, 1H), 7.43-7.62 (m, 4H), 7.20-7.35 (m, 2H), 6.81 (d, J=8.4 Hz, 1H), 2.85 (brt, J=7.6 Hz, 2H), 2.43 (t, J=7.6 Hz, 2H).

### Example 7: Synthesis of N-(8-methyl-2-oxo-1,2,3,4-tetrahydroquinolin-6-yl)-3-(m-tolyl)isonicotinamide (Compound 11)

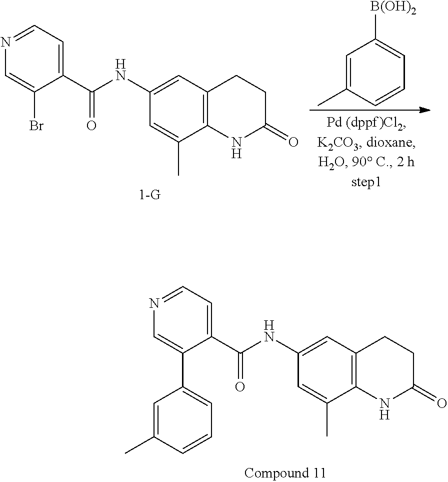

Step 1; To a solution of 3-bromo-N-(8-methyl-2-oxo-3,4-dihydro-1H-quinolin-6-yl)pyridine-4-carboxamide (100 mg, 277.62 umol, 1 eq) and m-tolylboronic acid (56.62 mg, 416.43 umol, 1.5 eq) in dioxane (3 mL) and H₂O (0.3 mL) was added K₂CO₃ (76.74 mg, 555.24 umol, 2 eq) and Pd(dppf)Cl₂ (20.31 mg, 27.76 umol, 0.1 eq). The mixture was stirred at 90° C. for 2 hr. LC-MS showed 3-bromo-N-(8-methyl-2-oxo-3,4-dihydro-1H-quinolin-6-yl)pyridine-4-carboxamide was consumed completely. The reaction mixture was diluted with H₂O 10 mL and extracted with EA (5 mL*3). The combined organic layers were washed with brine (5 mL*3), dried over Na₂SO₄, filtered and concentrated under reduced pressure to give a residue. The residue was purified by column chromatography (SiO2, Petroleum ether/Ethyl acetate=10/1 to 0/1). Compound N-(8-methyl-2-oxo-3,4-dihydro-1H-quinolin-6-yl)-3-(m-tolyl)pyridine-4-carboxamide (18 mg, 47.25 umol, 17.02% yield) was obtained as a solid. (M+H)⁺: 372.1. 1H NMR (400 MHz, METHANOL-d4) δ ppm 8.63-8.70 (m, 2H), 7.60 (d, J=5.2 Hz, 1H), 7.30-7.36 (m, 3H) 7.22 (br d, J=6.8 Hz, 1H), 7.14 (s, 1H), 7.06 (s, 1H), 2.89 (t, J=6.8 Hz, 2H), 2.52-2.56 (m, 2H) 2.36 (s, 3H) 2.21 (s, 3H).

### Example 8: Synthesis of N-(8-methyl-2-oxo-1,2,3,4-tetrahydroquinolin-6-yl)-3-(o-tolyl)isonicotinamide (Compound 15)

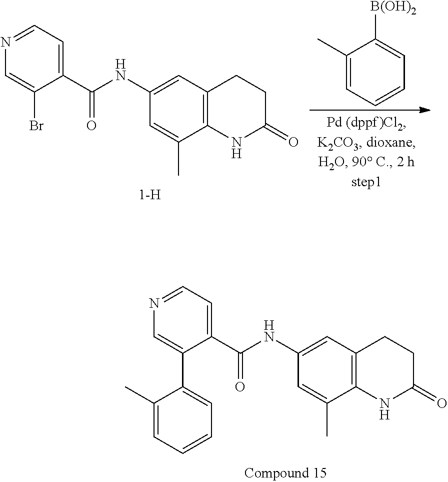

Step 1: To a solution of 3-bromo-N-(8-methyl-2-oxo-3,4-dihydro-1H-quinolin-6-yl)pyridine-4-carboxamide (100 mg, 277.62 umol, 1 eq) and o-tolylboronic acid (56.62 mg, 416.43 umol, 1.5 eq) in dioxane (3 mL) and H₂O (0.3 mL) was added K₂CO₃ (76.74 mg, 555.24 umol, 2 eq) and Pd(dppf)Cl₂ (20.31 mg, 27.76 umol, 0.1 eq). The mixture was stirred at 90° C. for 2 hr under N₂ atmosphere. The mixture was stirred at 90° C. for 4 hr under N₂ atmosphere. LCMS showed 3-bromo-N-(8-methyl-2-oxo-3,4-dihydro-1H-quinolin-6-yl)pyridine-4-carboxamide was consumed completely. Several new peaks were shown on LC-MS and 27.4% of desired compound was detected. The residue was purified by flash silica gel chromatography (ISCO®; 12 g SepaFlash® Silica Flash Column, Eluent of 0˜80% Ethyl acetate/Petroleum ethergradient @40 mL/min). Compound N-(8-methyl-2-oxo-3,4-dihydro-1H-quinolin-6-yl)-3-(o-tolyl)pyridine-4-carboxamide (18 mg, 48.46 umol, 17.46% yield) was obtained as a solid. (M+H)⁺: 372.2. ¹H NMR (400 MHz, METHANOL-d₄) δ ppm 8.69 (d, J=4.8 Hz, 1H), 8.51 (s, 1H), 7.66 (d, J=5.2 Hz, 1H), 7.23-7.31 (m, 4H), 7.09 (s, 1H), 7.01 (s, 1H), 2.83-2.89 (m, 2H), 2.49-2.55 (m, 2H), 2.18 (d, J=4.38 Hz, 6H).

### Example 9: Synthesis of 3-fluoro-N-(2-((8-methyl-2-oxo-1,2,3,4-tetrahydroquinolin-6-yl)oxy)ethyl)benzamide (Compound 8)

Compound 8 may be prepared according to the following scheme:

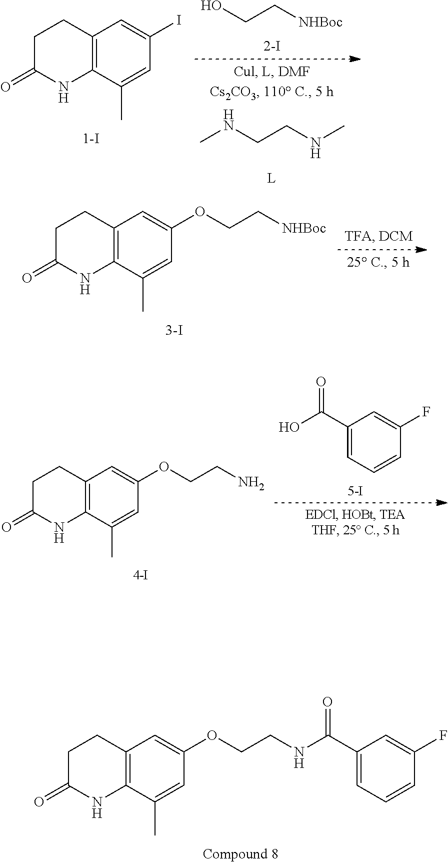

### Example 10: Synthesis of 3-fluoro-N-methyl-N-(2-((8-methyl-2-oxo-1,2,3,4-tetrahydroquinolin-6-yl)oxy)ethyl)benzamide (Compound 9)

Compound 9 may be prepared according to the following scheme:

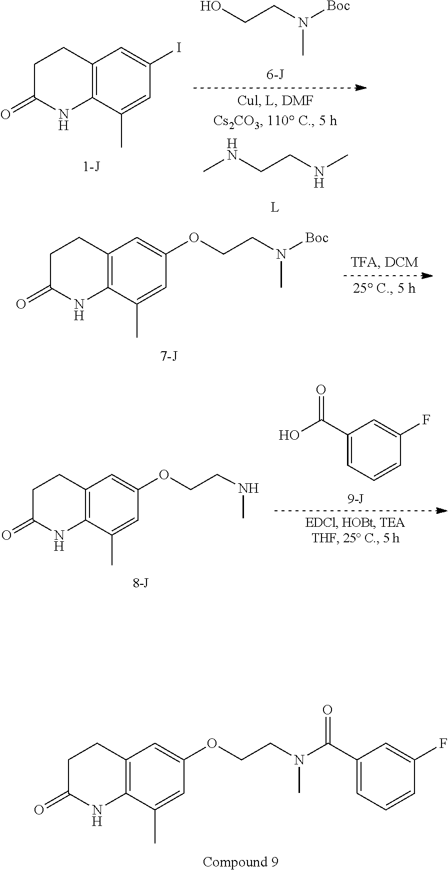

### Example 11: Synthesis of 6-(2-((3-fluorobenzyl)methyl)amino)ethoxy)-8-methyl-3,4-dihydroquinolin-2(1H)-one (Compound 10)

Compound 10 may be prepared according to the following scheme:

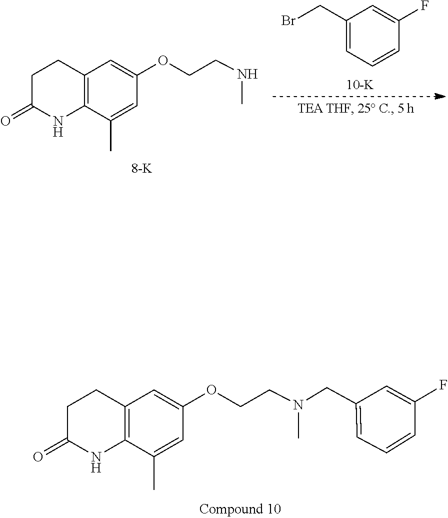 [SMILES: CNCCOc1cc(C)c2c(c1)CCC(=O)N2 ; Fc1cccc(CBr)c1 ; Cc1cc(OCCN(C)Cc2cccc(F)c2)cc2c1NC(=O)CC2]

### Example 12: Aldh1a3 Enzyme Inhibition Assay

Recombinant protein extraction: pET-Aldh1a3 transformed BL21-DE3 cultures induced at 20° C. for 19 h with 0.3 mM IPTG rocking. Cultures were spun at 3500 g for 10 min, supernatants were poured off and allowed to drain fully. Cells were resuspended in 10 mM HEPES pH 7.4, 10 mM KCl. Cells were freeze-thawed in liquid nitrogen and then a 37° C. water bath for 10 cycles followed by ultrasonication at 50% amplitude, 3 sec on, 9 sec off for 10 cycles at 4 V. Cell extracts were spun at 16000×g for 5 minutes.

Reaction performed at 20° C. in reaction buffer (10 mM HEPES pH 7.4, 10 mM KCl, 0.1 M Resazurin, 1 mg/mL BSA, 200 uM NAD+, diaphorase and aldehyde substrate). Recombinant enzyme and inhibitor added immediately before assay. Reaction rate measured by resorufin fluorescence.

The IC₅₀ values of selected tested compounds are shown in Table 2 below.

TABLE 2
IC₅₀ values* for inhibition of hALDH1a3 and mALDH1a3

| No. | hAldh1a3 IC50 | mAldh1a3 IC50 |
|---|---|---|
| 1 | D | D |
| 2 | D | No activity |
| 3 | D | No activity |
| 4 | B | C |
| 5 | A | B |
| 6 | A | B |
| 7 | B | B |
| 8 | No activity | No activity |
| 9 | E | E |
| 10 | D | D |
| 11 | No activity | No activity |
| 12 | No activity | No activity |
| 13 | No activity | No activity |
| 14 | No activity | No activity |
| 15 | No activity | No activity |
| 16 | No activity | No activity |
| 18 | No activity | No activity |
| 19 | No activity | No activity |
| 20 | D | D |
| 21 | E | No activity |
| 22 | E | No activity |
| 23 | E | E |
| 24 | D | E |
| 25 | E | No activity |
| 26 | D | No activity |
| 27 | A | C |
| 28 | E | D |
| 31 | B | A |
| 32 | B | A |
| 33 | B | A |
| 34 | A | A |
| 35 | B | A |
| 36 | B | A |
| 37 | B | A |
| 38 | B | A |
| 39 | C | C |
| 40 | A | A |
| 41 | A | A |
| 42 | B | A |
| 43 | B | A |
| 44 | D | B |
| 45 | D | B |
| 46 | B | A |
| 48 | B | A |
| 49 | A | A |
| 50 | C | A |
| 52 | B | A |
| 56 | C | B |
| 57 | C | B |
| 59 | D | C |
| 60 | C | B |
| 61 | B | C |
| 62 | D | D |
| 63 | A | A |
| 64 | C | D |
| 65 | B | C |
| 66 | B | B |
| 67 | C | C |
| 72 | B | B |
| 73 | A | A |
| 75 | A | B |
| 77 | C | B |
| 78 | A | A |
| 79 | B | A |
| 80 | A | A |
| 81 | B | A |
| 82 | B | B |
| 83 | B | A |

*The IC₅₀ values are reported herein according to the Activity Level: A < 100 nM; B: 100 nM − 250 nM; C: 250 nM − 1 μM (micromolar); D: 1 μM − 5 μM; E: >5 μM.

### Example 13: Aldh1a2 Enzyme Inhibition Assay

Recombinant protein extraction: pET-Aldh1a2 transformed BL21-DE3 cultures induced at 20° C. for 19 h with 0.3 mM IPTG rocking. Cultures were spun at 3500 g for 10 min, supernatants were poured off and allowed to drain fully. Cells were resuspended in 10 mM HEPES pH 7.4, 10 mM KCl. Cells were freeze-thawed in liquid nitrogen and then a 37° C. water bath for 10 cycles followed by ultrasonication at 50% amplitude, 3 sec on, 9 sec off for 10 cycles at 4° C. Cell extracts were spun at 16000×g for 5 minutes.

Reaction performed at 20° C. in reaction buffer (10 mM HEPES pH 7.4, 10 mM KCl, 0.1 M Resazurin, 1 mg/mL BSA, 200 μM NAD+, diaphorase and aldehyde substrate). Recombinant enzyme and inhibitor added immediately before assay. Reaction rate measured by resorufln fluorescence.

The IC₅₀ values of selected tested compounds are shown in Table 3 below.

TABLE 3
IC₅₀ values* for inhibition of hALDH1a2 and mALDH1a2

| No. | hAldh1a2 IC50 | mAldh1a2 IC50 |
|---|---|---|
| 1 | B | B |
| 2 | B | C |
| 3 | C | C |
| 4 | C | C |
| 5 | D | D |
| 6 | D | D |
| 7 | C | C |
| 8 | No activity | No activity |
| 9 | D | D |
| 10 | D | D |
| 11 | D | D |
| 12 | D | E |
| 13 | E | E |
| 14 | E | E |
| 15 | E | E |
| 16 | E | E |
| 18 | D | D |
| 19 | D | D |
| 20 | A | B |
| 21 | D | D |
| 22 | E | E |
| 23 | D | D |
| 24 | B | C |
| 25 | D | D |
| 26 | B | B |
| 27 | B | B |
| 28 | E | E |
| 31 | C | C |
| 32 | C | C |
| 33 | A | A |
| 34 | A | A |
| 35 | C | C |
| 36 | C | C |
| 37 | B | B |
| 38 | B | B |
| 39 | C | C |
| 40 | A | A |
| 41 | A | A |
| 42 | C | D |
| 43 | D | D |
| 44 | D | D |
| 45 | C | C |
| 46 | B | B |
| 48 | B | B |
| 49 | A | B |
| 50 | C | C |
| 52 | A | B |
| 56 | D | D |
| 57 | D | D |
| 59 | D | D |
| 60 | C | C |
| 61 | C | C |
| 62 | E | E |
| 63 | B | C |
| 64 | C | D |
| 65 | D | D |
| 66 | C | C |
| 67 | D | D |
| 72 | B | C |
| 73 | B | A |
| 75 | B | C |
| 77 | C | C |
| 78 | C | D |
| 79 | C | C |
| 80 | B | C |
| 81 | C | C |
| 82 | B | B |
| 83 | A | A |

*The IC₅₀ values are reported herein according to the Activity Level: A < 100 nM; B: 100 nM − 250 nM; C: 250 nM − 1 μM (micromolar); D: 1 μM − 5 μM; E: >5 μM.

1. A compound of Formula (III) 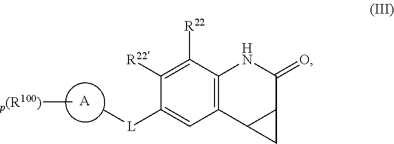
or a pharmaceutically acceptable salt or deuterated form thereof,
wherein:
ring A is heteroaryl, heterocyclyl, or aryl;
L is —C(O)—NH—, —C(NR^{A})—NH—, —C(O)—N(R^{A})-alkylene-O—, or -(alkylene)-N(R^{A})-alkylene-O—;
R²² is halo, —CN, —C₁₋₆ alkyl, —C₁₋₆ alkyl-CN, —C₁₋₆ haloalkyl, -carbocyclyl, or —C₁₋₆ alkyl-carbocyclyl;
R^{22′} is H, —CN, halo, —C₁₋₆alkyl, —C₁₋₆haloalkyl, —C₁₋₆ alkyl-CN, carbocyclyl, or —C₁₋₆ alkyl-carbocyclyl;
p is 0, 1, or 2;
each R¹⁰⁰ is independently halo, —CN, hydroxy, —C₁₋₆ alkyl, —C₁₋₆ alkylene-carbocyclyl, —C₁₋₆alkylene-heterocyclyl, —C₁₋₆haloalkyl, carbocyclyl, aryl, or heteroaryl, wherein aryl or heteroaryl is optionally substituted with 1 or 2 R¹⁰¹;
each R¹⁰¹ is independently halo, —CN, hydroxy, or —C₁₋₆ alkyl; and
R^{A} is H or —C₁₋₆ alkyl
wherein the compound is not

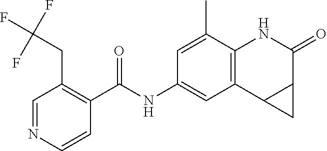 [SMILES: Cc1cc(NC(=O)c2ccncc2CC(F)(F)F)cc2c1NC(=O)C1CC21]

2. The compound of claim 1, wherein the compound of Formula (III) is a compound of Formula (IIIA): 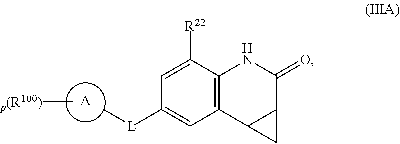
or a pharmaceutically acceptable salt or deuterated form thereof,
wherein:
ring A is heteroaryl or aryl;
L is —C(O)—NH—, —C(NR^{A})—NH—, —C(O)—N(R^{A})-alkylene-O—, or -(alkylene)-N(R^{A})-alkylene-O—;
R²² is halo, —CN, —C₁₋₆ alkyl, —C₁₋₆ alkyl-CN, —C₁₋₆ haloalkyl, -carbocyclyl, or —C₁₋₆ alkyl-carbocyclyl;
p is 0, 1, or 2;
each R¹⁰⁰ is independently halo, —C₁₋₆alkyl, —C₁₋₆alkylene-carbocyclyl, —C₁₋₆ alkylene-heterocyclyl, —C₁₋₆ haloalkyl, or aryl, wherein aryl is optionally substituted with 1 or 2 R¹⁰¹;
each R¹⁰¹ is independently halo, —CN, hydroxy, or —C₁₋₆ alkyl; and
R^{A} is H or —C₁₋₆ alkyl,
wherein the compound is not

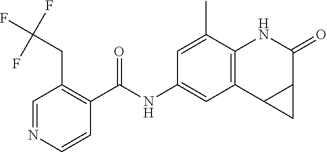 [SMILES: Cc1cc(NC(=O)c2ccncc2CC(F)(F)F)cc2c1NC(=O)C1CC21]

3. The compound of claim 1 or 2, wherein L is —C(O)—NH—, —C(O)—N(R^{A})-alkylene-O—, or -(alkylene)-N(R^{A})-alkylene-O—.

4. The compound of claim 1, 2 or 3, wherein L is —C(O)—NH—.

5. The compound of any one of claims 1-4, wherein ring A is a 5 or 6-membered heteroaryl, phenyl, a 5,6-bicyclic heteroaryl, a 5,6-bicyclic heterocyclyl, a 6,6-bicyclic heterocyclyl, a 6,6-bicyclic heteroaryl, or a 3-8 membered heterocyclyl.

6. The compound of claim 5, wherein ring A is a 5-membered heteroaryl, 6-membered heteroaryl, or phenyl.

7. The compound of any one of claims 1-6, wherein ring A is pyridyl, phenyl, thiazolyl, isothiazolyl, imidazolyl, oxazolyl, thiadiazolyl, or isoxazolyl.

8. The compound of any one of claims 1-7, wherein ring A is

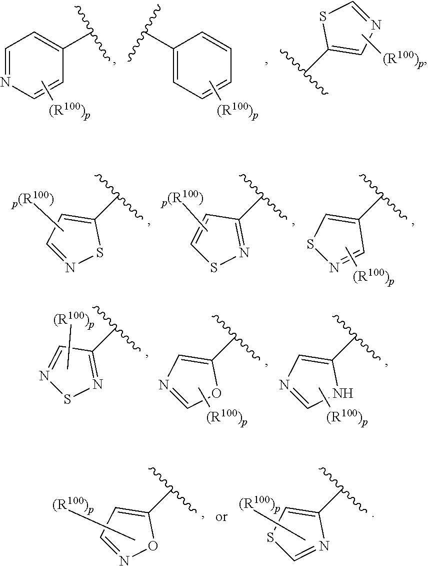

9. The compound of any one of claims 1-8, wherein ring A-(R¹⁰⁰)ₚ is:

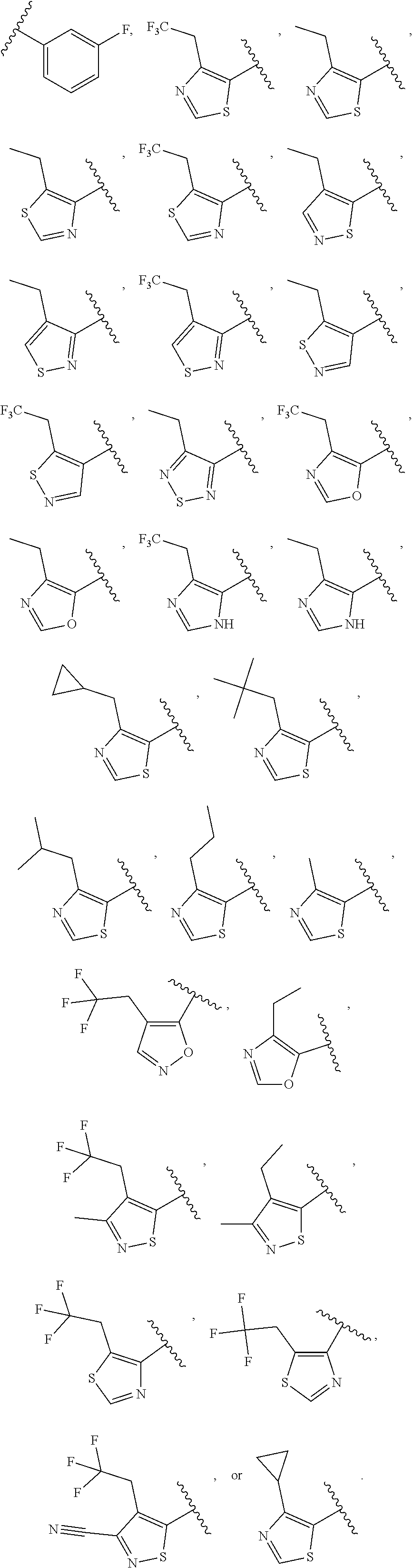

10. The compound of any one of claims 1-8, wherein ring A-(R¹⁰⁰)ₚ is:

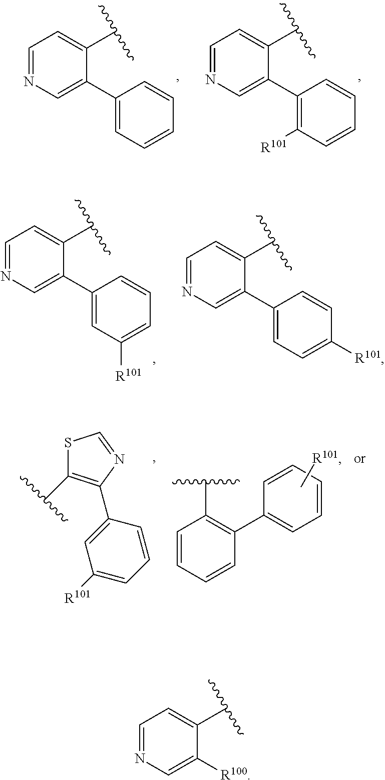

11. A compound of Formula (IV): 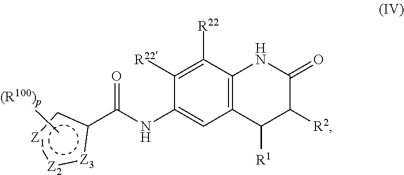
or a pharmaceutically acceptable salt or deuterated form thereof,
wherein:
R¹ is H and R² is H, or R¹ and R² are joined to form a carbocyclyl;
R²² is halo, —CN, —C₁₋₆alkyl, —C₁₋₆ alkyl-CN, —C₁₋₆haloalkyl, carbocyclyl, or —C₁₋₆ alkyl-carbocyclyl;
R^{22′} is H, —CN, halo, —C₁₋₆ alkyl, —C₁₋₆ haloalkyl, —C₁₋₆alkyl-CN, carbocyclyl, or —C₁₋₆ alkyl-carbocyclyl;
p is 0, 1, or 2;
each R¹⁰⁰ is independently halo, —CN, hydroxy, —C₁₋₆ alkylene-carbocyclyl, —C₁₋₆ alkyl, —C₁₋₆ haloalkyl, or carbocyclyl;
Z₁, Z₂, and Z₃ are independently selected from —CH—, —S—, —N—, —NH—, and —O—; and
the dashed line represents a double or is absent to provide an aromatic ring;
wherein the compound is not

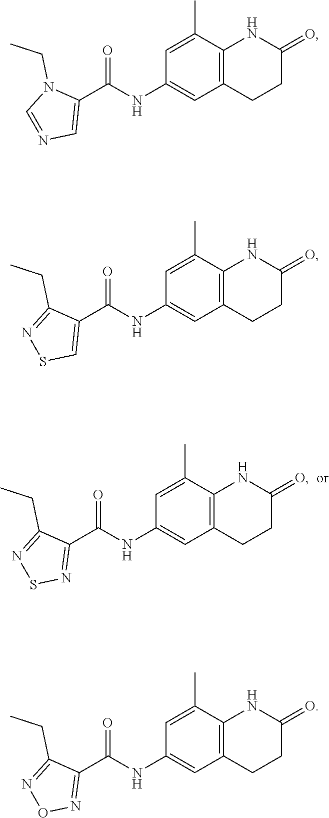

12. The compound of claim 11, wherein the compound of Formula (IV) is a compound of Formula (IVA): 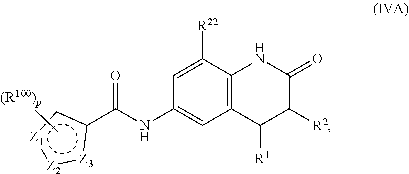
or a pharmaceutically acceptable salt or deuterated form thereof,
wherein:
R¹ is H and R² is H, or R¹ and R² are joined to form a carbocyclyl;
R²² is halo, —CN, —C₁₋₆alkyl, —C₁₋₆ alkyl-CN, —C₁₋₆haloalkyl, carbocyclyl, or —C₁₋₆ alkyl-carbocyclyl;
p is 0, 1, or 2;
each R¹⁰⁰ is independently halo, —CN, hydroxy, —C₁₋₆ alkylene-carbocyclyl, —C₁₋₆ alkyl, —C₁₋₆ haloalkyl, or carbocyclyl;
Z₁, Z₂, and Z₃ are independently selected from CH, S, N, NH, and O; and
dashed line represents a double or is absent to provide an aromatic ring,
wherein the compound is not

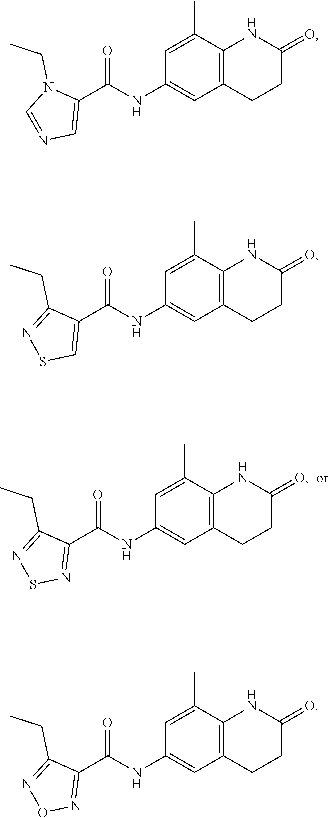

13. The compound of claim 12, wherein:

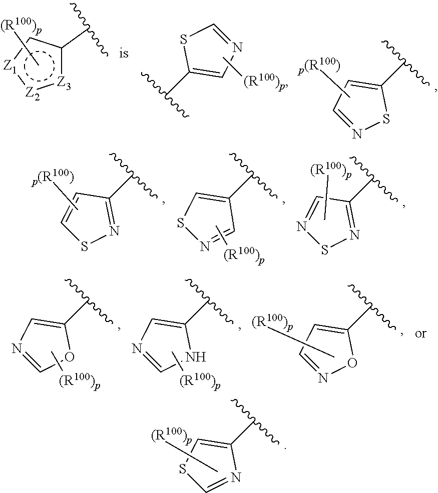

14. The compound of anyone of claims 1-12, wherein each R¹⁰⁰ is independently —C₁₋₆ alkyl, or —C₁₋₆ haloalkyl, carbocyclyl, or —C₁₋₆ alkylene-carbocyclyl.

15. The compound of claim 13 or 14, wherein:

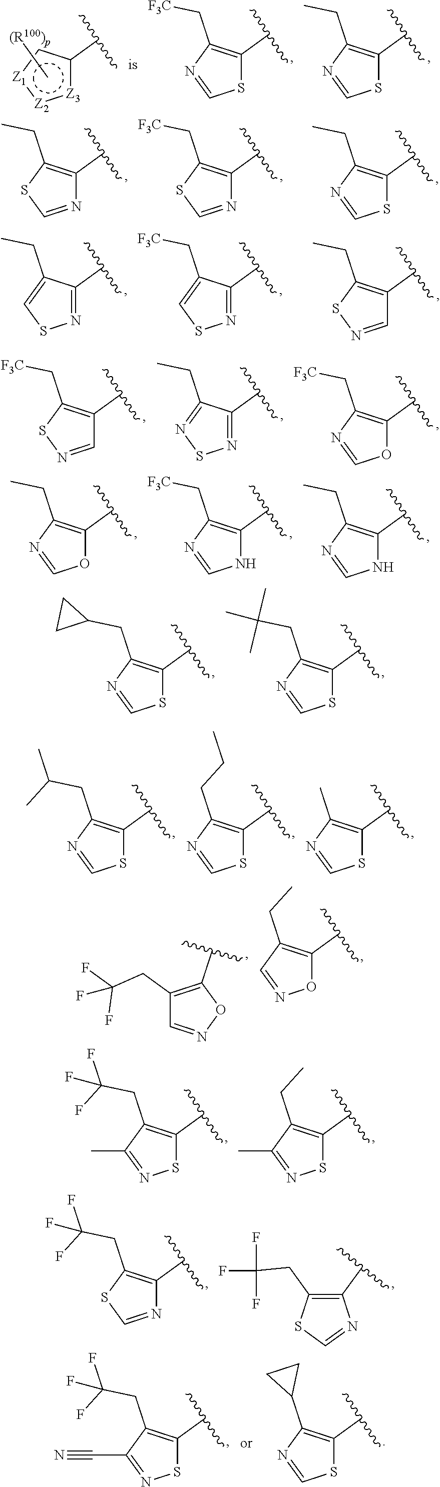

16. The compound of any one of claims 1-13, wherein each R¹⁰⁰ is independently halo, —CN, —C₁₋₆haloalkyl, —C₁₋₆ alkylene-carbocyclyl, —C₁₋₆ alkyl, carbocyclyl, aryl, or heteroaryl, wherein aryl or heteroaryl is optionally substituted with 1 or 2 R¹⁰¹.

17. The compound of any one of claims 1-9 or 16, wherein p is 1 and R¹⁰⁰ is aryl, wherein aryl is optionally substituted with 1 or 2 R¹⁰¹.

18. A compound of Formula (V): 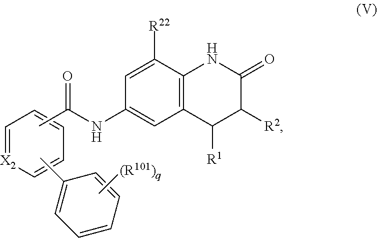
or a pharmaceutically acceptable salt or deuterated form thereof,
wherein:
R¹ is H and R² is H, or R¹ and R² are joined to form a carbocyclyl;
R²² is halo, —CN, —C₁₋₆ alkyl, —C₁₋₆ alkyl-CN, —C₁₋₆ haloalkyl, carbocyclyl, or —C₁₋₆ alkyl-carbocyclyl;
q is 0, 1, or 2;
each R¹⁰¹ is independently halo, —CN, hydroxy, —C₁₋₆alkyl, or —C₁₋₆ haloalkyl; and
X₂ is —CH— or —N—.

19. The compound of claim 18, wherein

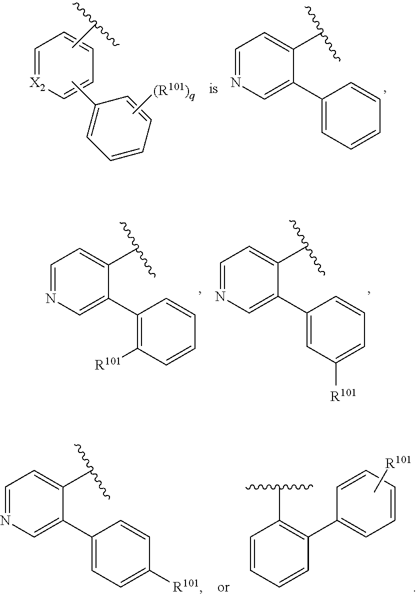

20. The compound of any one of the preceding claims, wherein R¹⁰¹ is fluoro, hydroxy, —CN, or methyl.

21. The compound of any one of claims 1-20, wherein R²² is halo, —CN, —C₁₋₆ alkyl, or C₁₋₆ haloalkyl.

22. The compound of claim 21, wherein R²² is halo or —C₁₋₆ alkyl.

23. The compound of claim 22, wherein R²² is methyl or chloro.

24. The compound of claim 1 or 11, wherein R^{22′} is —H, halo, —C₁₋₆ alkyl, or —C₁₋₆ haloalkyl.

25. The compound of claim 24, wherein R^{22′} is —H, —F, or —Cl.

26. The compound of claim 25, wherein R^{22′} is —H.

27. A compound selected from:

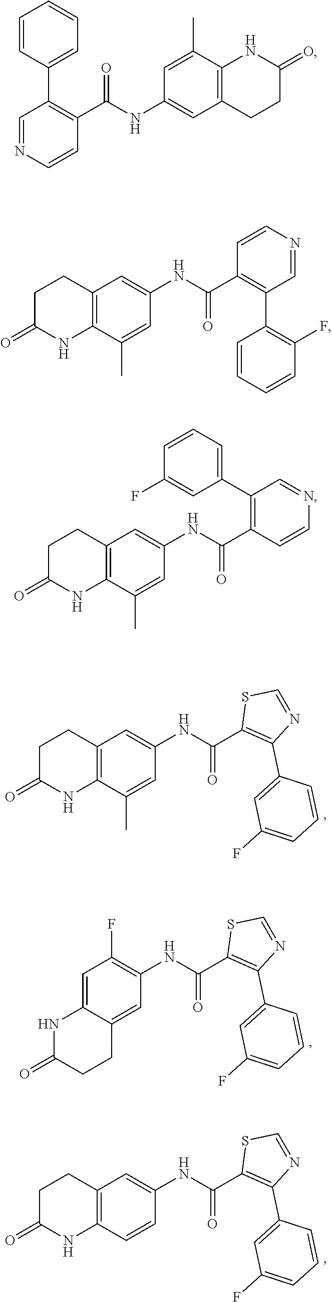 [SMILES: Cc1cc(NC(=O)c2ccncc2-c2ccccc2)cc2c1NC(=O)CC2 ; Cc1cc(NC(=O)c2ccncc2-c2ccccc2F)cc2c1NC(=O)CC2 ; Cc1cc(NC(=O)c2ccncc2-c2cccc(F)c2)cc2c1NC(=O)CC2 ; Cc1cc(NC(=O)c2scnc2-c2cccc(F)c2)cc2c1NC(=O)CC2 ; O=C1CCc2cc(NC(=O)c3scnc3-c3cccc(F)c3)c(F)cc2N1 ; O=C1CCc2cc(NC(=O)c3scnc3-c3cccc(F)c3)ccc2N1]

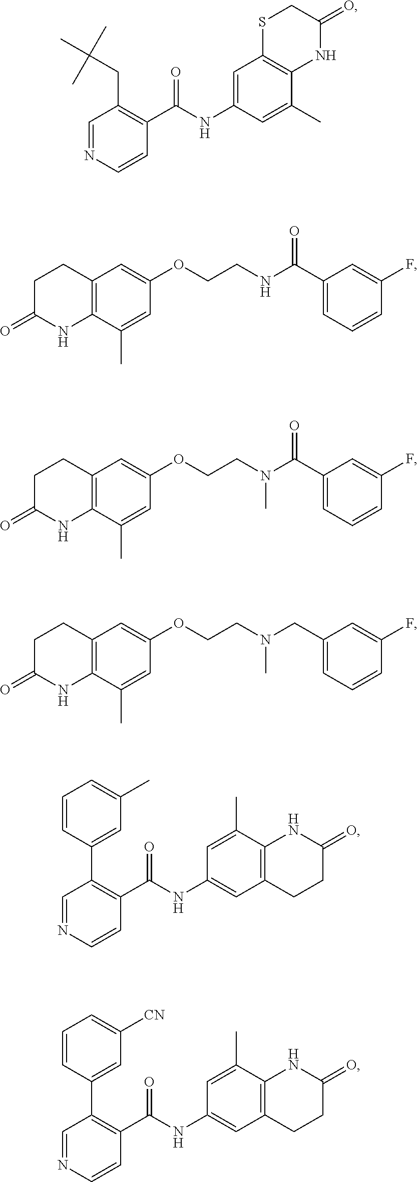 [SMILES: Cc1cc(NC(=O)c2ccncc2CC(C)(C)C)cc2c1NC(=O)CS2 ; Cc1cc(OCCNC(=O)c2cccc(F)c2)cc2c1NC(=O)CC2 ; Cc1cc(OCCN(C)C(=O)c2cccc(F)c2)cc2c1NC(=O)CC2 ; Cc1cc(OCCN(C)Cc2cccc(F)c2)cc2c1NC(=O)CC2 ; Cc1cccc(-c2cnccc2C(=O)Nc2cc(C)c3c(c2)CCC(=O)N3)c1 ; Cc1cc(NC(=O)c2ccncc2-c2cccc(C#N)c2)cc2c1NC(=O)CC2]

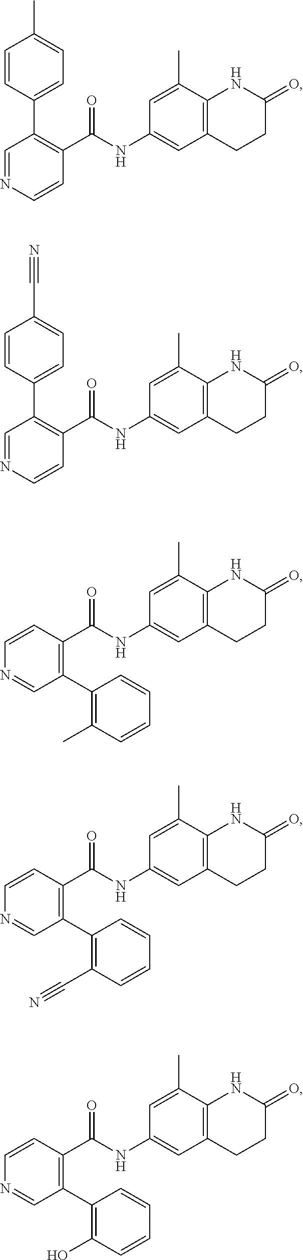 [SMILES: Cc1ccc(-c2cnccc2C(=O)Nc2cc(C)c3c(c2)CCC(=O)N3)cc1 ; Cc1cc(NC(=O)c2ccncc2-c2ccc(C#N)cc2)cc2c1NC(=O)CC2 ; Cc1ccccc1-c1cnccc1C(=O)Nc1cc(C)c2c(c1)CCC(=O)N2 ; Cc1cc(NC(=O)c2ccncc2-c2ccccc2C#N)cc2c1NC(=O)CC2 ; Cc1cc(NC(=O)c2ccncc2-c2ccccc2O)cc2c1NC(=O)CC2]

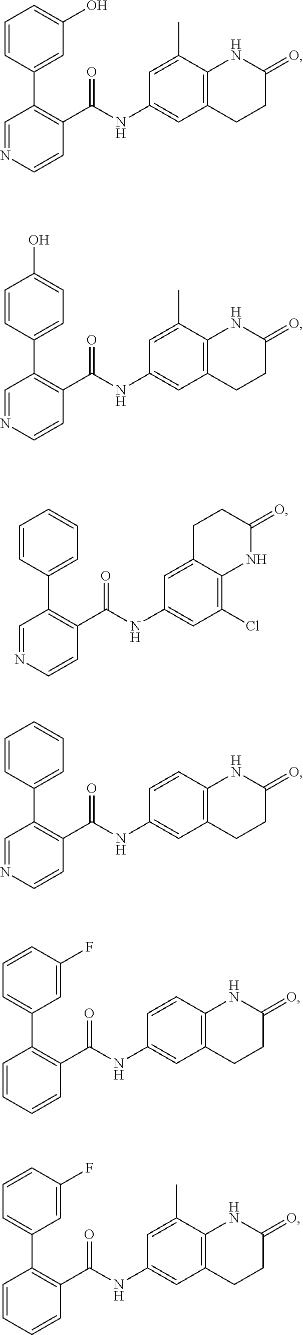 [SMILES: Cc1cc(NC(=O)c2ccncc2-c2cccc(O)c2)cc2c1NC(=O)CC2 ; Cc1cc(NC(=O)c2ccncc2-c2ccc(O)cc2)cc2c1NC(=O)CC2 ; O=C1CCc2cc(NC(=O)c3ccncc3-c3ccccc3)cc(Cl)c2N1 ; O=C1CCc2cc(NC(=O)c3ccncc3-c3ccccc3)ccc2N1 ; O=C1CCc2cc(NC(=O)c3ccccc3-c3cccc(F)c3)ccc2N1 ; Cc1cc(NC(=O)c2ccccc2-c2cccc(F)c2)cc2c1NC(=O)CC2]

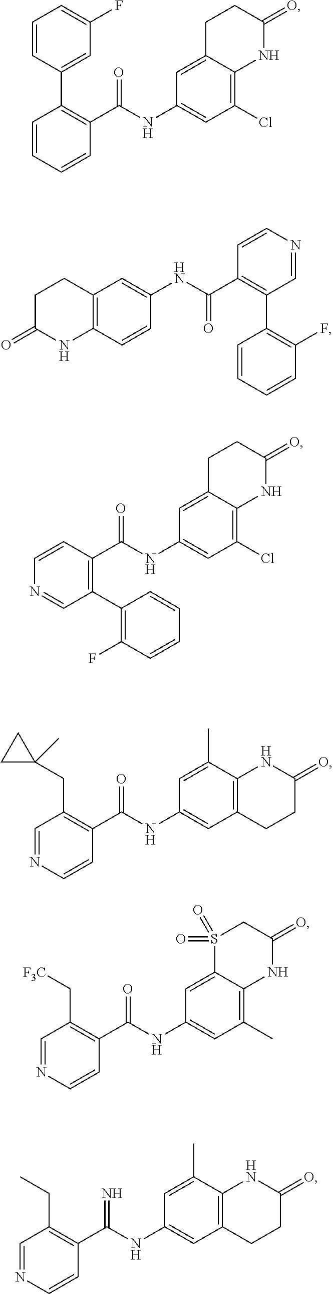 [SMILES: O=C1CCc2cc(NC(=O)c3ccccc3-c3cccc(F)c3)cc(Cl)c2N1 ; O=C1CCc2cc(NC(=O)c3ccncc3-c3ccccc3F)ccc2N1 ; O=C1CCc2cc(NC(=O)c3ccncc3-c3ccccc3F)cc(Cl)c2N1 ; Cc1cc(NC(=O)c2ccncc2CC2(C)CC2)cc2c1NC(=O)CC2 ; Cc1cc(NC(=O)c2ccncc2CC(F)(F)F)cc2c1NC(=O)CS2(=O)=O ; CCc1cnccc1C(=N)Nc1cc(C)c2c(c1)CCC(=O)N2]

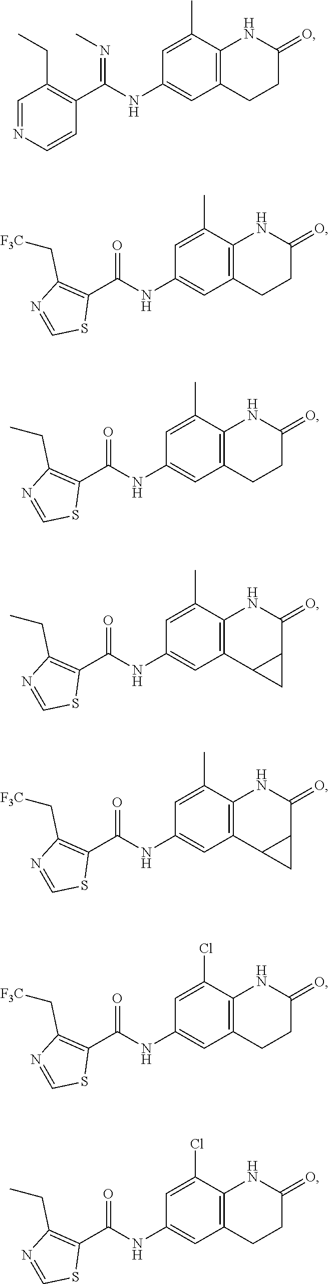

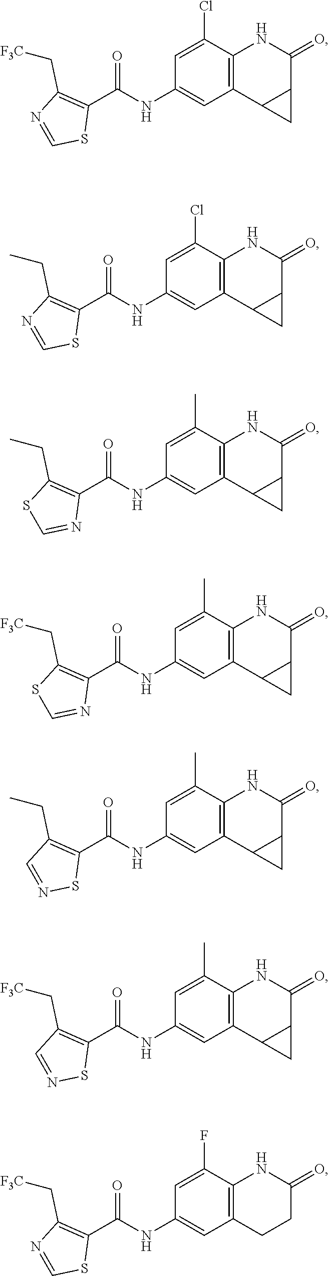

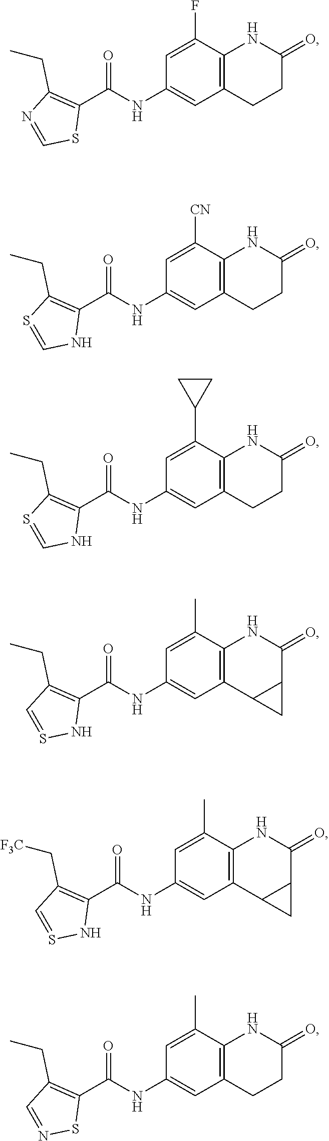

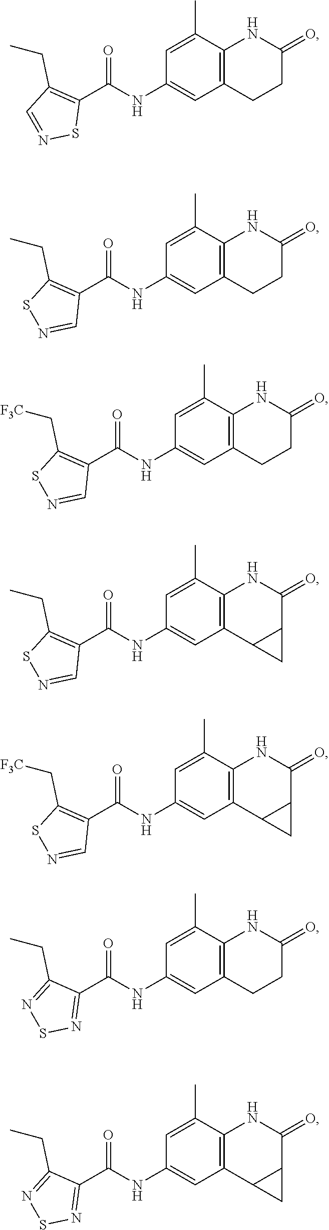

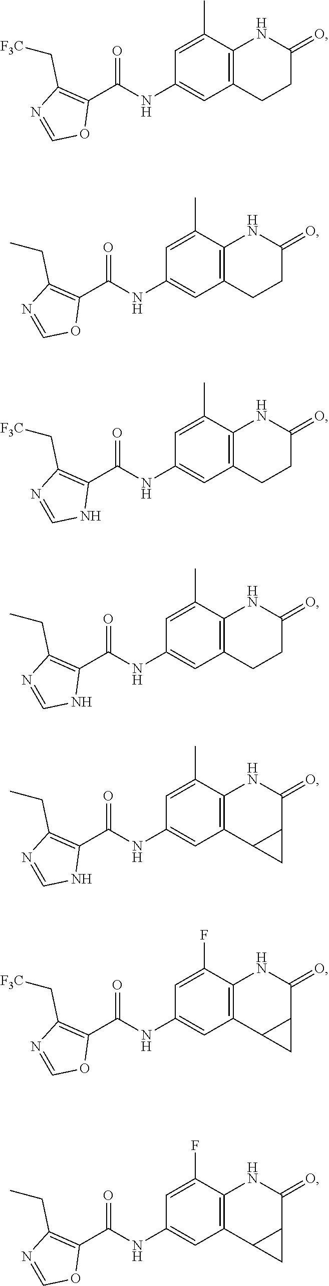

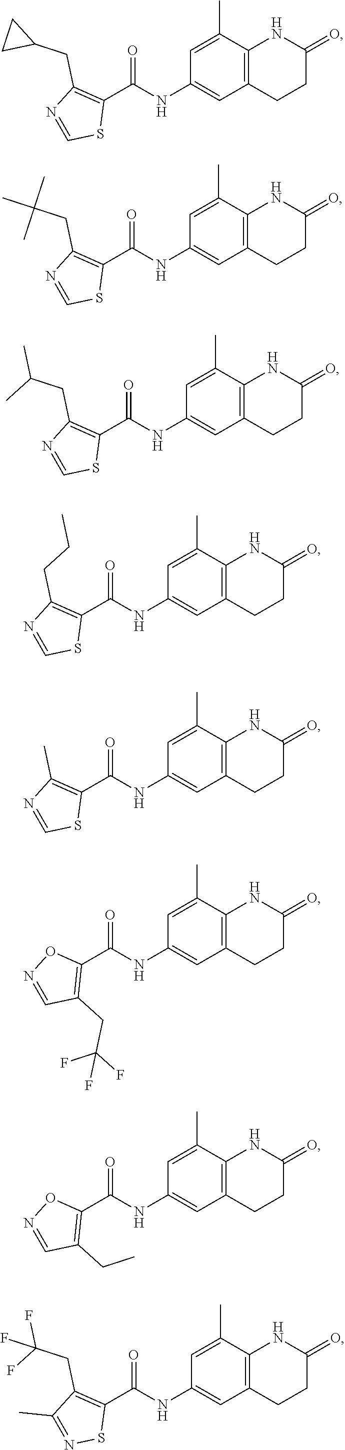

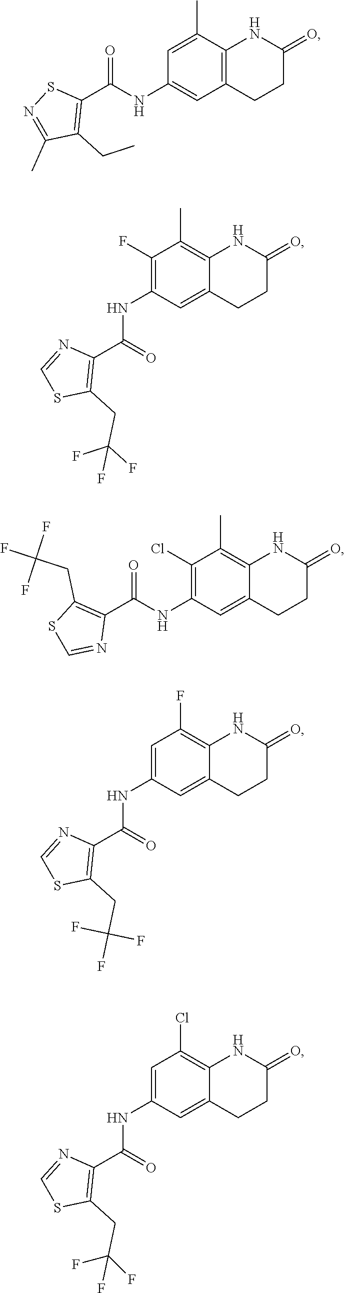 [SMILES: CCc1c(C)nsc1C(=O)Nc1cc(C)c2c(c1)CCC(=O)N2 ; Cc1c(F)c(NC(=O)c2ncsc2CC(F)(F)F)cc2c1NC(=O)CC2 ; Cc1c(Cl)c(NC(=O)c2ncsc2CC(F)(F)F)cc2c1NC(=O)CC2 ; O=C1CCc2cc(NC(=O)c3ncsc3CC(F)(F)F)cc(F)c2N1 ; O=C1CCc2cc(NC(=O)c3ncsc3CC(F)(F)F)cc(Cl)c2N1]

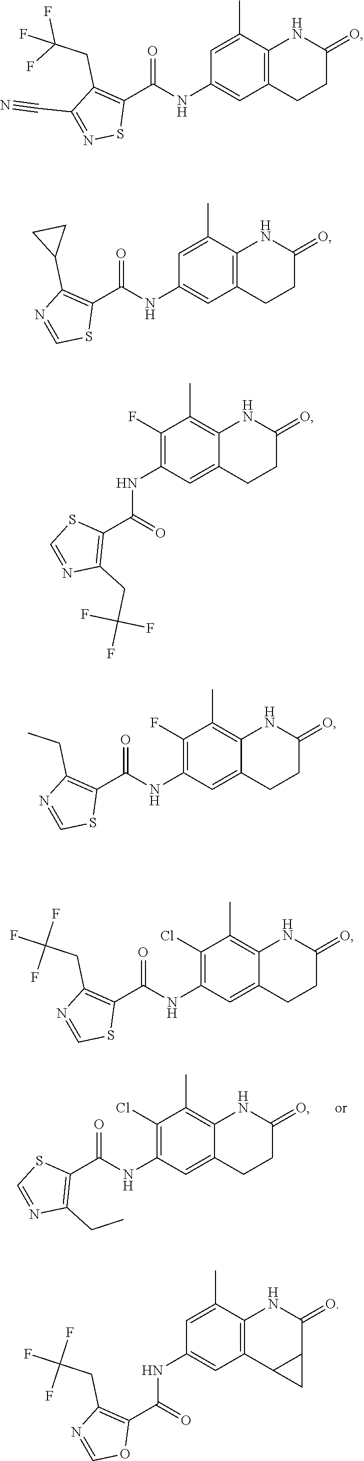

28. A pharmaceutical composition comprising the compound of any one of claims 1-27, or a pharmaceutically acceptable salt thereof, and a pharmaceutically acceptable carrier.

29. A method of treating cancer in a subject in need thereof, the method comprising administering to the subject a therapeutically effective amount of the compound of any one of claims 1-27 or a pharmaceutically acceptable salt thereof, or the pharmaceutical composition of claim 28.

30. A method of treating metastatic cancer or chemoresistant cancer in a subject in need thereof, the method comprising administering to the subject a therapeutically effective amount of the compound of any one of claim 1-27 or a pharmaceutically acceptable salt thereof, or the pharmaceutical composition of claim 28.

31. A method of treating or preventing metastasis of a cancer in a subject in need thereof, the method comprising administering to the subject an effective amount of the compound of any one of claims 1-27 or a pharmaceutically acceptable salt thereof, or the pharmaceutical composition of claim 28.

32. A method of sensitizing cancer for chemotherapy in a subject in need thereof, the method comprising administering to the subject an effective amount of the compound of any one of claims 1-27 or a pharmaceutically acceptable salt thereof, or the pharmaceutical composition of claim 28.

33. The method of any one of claims 29-32, further comprising administering to the subject an effective amount of a second anti-cancer therapy.

34. The method of claim 33, wherein the second anti-cancer therapy is a chemotherapeutic agent, a receptor tyrosine kinase inhibitor, or a therapeutic antibody.

35. The method of any one of claims 29-34, wherein the cancer is a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, sarcoma, gastrointestinal stromal tumor, peripheral nerve sheath tumor, myeloma, and/or endometrial cancer.

36. A method of treating or preventing type 2 diabetes in a subject in need thereof, the method comprising administering to the subject an effective amount of the compound of any one of claims 1-27 or a pharmaceutically acceptable salt thereof, or the pharmaceutical composition of claim 28.

37. A method of treating or preventing a metabolic disease in a subject in need thereof, the method comprising administering to the subject an effective amount of the compound of any one of claims 1-27 or a pharmaceutically acceptable salt thereof, or the pharmaceutical composition of claim 28.

38. A method of inhibiting an aldehyde dehydrogenase in a subject in need thereof, comprising administering to the subject an effective amount of the compound of any one of claims 1-27 or a pharmaceutically acceptable salt thereof, or the pharmaceutical composition of claim 28.

39. A method of treating a disease or disorder associated with aldehyde dehydrogenase, preferably, a disease or disorder associated with aldehyde dehydrogenase isoform 1a3 (ALDH1a3) and/or (ALDH1a2) in a subject in need thereof, comprising administering to the subject an effective amount of the compound of any one of claims 1-27, or a pharmaceutically acceptable salt thereof, or the pharmaceutical composition of claim 28.

40. The method of claim 39, wherein the disease or disorder is a proliferative disease or disorder or a metabolic disease or disorder.

41. A method of treating an endothelial cell or smooth muscle cell disease or disorder, such as pulmonary arterial hypertension or neointimal hyperplasia in a subject in need thereof, comprising administering to the subject an effective amount of the compound of any one of claims 1-27 or a pharmaceutically acceptable salt thereof, or the pharmaceutical composition of claim 28.

42. A method of antagonizing the retinoid pathway in a subject in need thereof, the method comprising administering to the subject an effective amount of the compound of any one of claims 1-27 or a pharmaceutically acceptable salt thereof, or the pharmaceutical composition of claim 28.

43. A method of treating a cancer in a subject in need thereof, the method comprising administering to the subject a therapeutically effective amount of the compound of any one of claims 1-27 or a pharmaceutically acceptable salt thereof, or the pharmaceutical composition of claim 28, in combination with an immunotherapy.

44. The method of claim 43, wherein the immunotherapy comprises administering to the subject an anti-PD-1 antibody, anti-PD-L1 antibody, anti-CTLA4 antibody, IL-2, autologous T cell therapy, bispecific antibody therapy, anti-TGFβ antibody, a JAK/STAT inhibitor, or any combination thereof.

45. The method of claim 43 or 44, wherein the cancer is a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, sarcoma, gastrointestinal stromal tumor, peripheral nerve sheath tumor, myeloma, and/or endometrial cancer.

46. A method of treating a cancer in a subject in need thereof, wherein the cancer is unresponsive to one or more immunotherapy or the subject has developed resistance to one or more immunotherapy, the method comprising administering to the subject a therapeutically effective amount of the compound of any one of claims 1-27 or a pharmaceutically acceptable salt thereof, or the pharmaceutical composition of claim 28, and optionally administering to the subject an immunotherapy.

47. The method of claim 46, wherein the cancer is unresponsive to treatment with anti-PD-1 or anti-PD-L1 antibodies.

48. The method of claim 46, wherein the subject has developed resistance to anti-PD-1 or anti-PD-L1 antibodies-based treatment.

49. The method of any one of claims 46-48, comprising administering to the subject an anti-PD-1 antibody, anti-PD-L1 antibody, anti-CTLA4 antibody, IL-2, autologous T cell therapy, bispecific antibody therapy, anti-TGFβ antibody, a JAK/STAT inhibitor, or any combination thereof.

50. The method of any one of claims 46-49, wherein the cancer is a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, sarcoma, gastrointestinal stromal tumor, peripheral nerve sheath tumor, myeloma, and/or endometrial cancer.

51. A method of treating a disease or disorder associated with retinoid pathway activation in a subject in need thereof, the method comprising administering to the subject a therapeutically effective amount of the compound of any one of claims 1-27 or a pharmaceutical salt thereof, or the pharmaceutical composition of claim 28.

52. The method of claim 51, wherein the disease or disorder is associated with immune tolerance, induction of T_{reg} cells and/or M2 macrophages, and/or effector T cell suppression.

53. The method of claim 51 or 52, wherein the disease or disorder is cancer.

54. The method of claim 53, wherein the cancer is a breast cancer, colorectal cancer, kidney cancer, ovarian cancer, gastric cancer, thyroid cancer, urothelial cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, hematologic cancer, sarcoma, gastrointestinal stromal tumor, peripheral nerve sheath tumor, myeloma, and/or endometrial cancer.

55. The method of any one of claims 51-54, further comprising administering to the subject an immunotherapy (e.g., an immune checkpoint inhibitor).

56. The method of claim 55, wherein administering the immunotherapy comprises administering to the subject an anti-PD-1 antibody, anti-PD-L1 antibody, anti-CTLA4 antibody, anti-LAG-3, anti-TIGIT, IL-2, autologous T cell therapy, bispecific antibody therapy, anti-TGFβ antibody, a JAK/STAT inhibitor, or any combination thereof.

57. A method of male contraception in a subject in need thereof, the method comprising administering to the subject an effective amount of the compound of any one of claims 1-27 or a pharmaceutically acceptable salt thereof, or the pharmaceutical composition of claim 28.

58. A method of inhibiting T_{reg} cell and/or M2 macrophage formation in a subject in need thereof, the method comprising administering to the subject an effective amount of the compound of any one of claims 1-27 or a pharmaceutical salt thereof, or the pharmaceutical composition of claim 28.

59. The method of claim 58, wherein the subject is characterized as having a cancer unresponsive to one or more immunotherapy or the subject has developed resistance to one or more immunotherapy.

60. The method of claim 39, wherein ALDH1a2 is selectively inhibited.
